(19) United States
(12) Patent Application Publication
(10) Pub. No.: US 2019/0374560 A1
(43) Pub. Date: Dec. 12, 2019

(54) SELECTIVE DRUG DELIVERY COMPOSITIONS AND METHODS OF USE

(71) Applicant: Avelas Biosciences, Inc., La Jolla, CA (US)

(72) Inventors: Jesus GONZALEZ, Carlsbad, CA (US); Junjie LIU, San Diego, CA (US); Marcel MIAMPAMBA, San Diego, CA (US)

(21) Appl. No.: 16/450,836

(22) Filed: Jun. 24, 2019

Related U.S. Application Data
(63) Continuation of application No. 14/786,402, filed on Oct. 22, 2015.
(60) Provisional application No. 61/814,771, filed on Apr. 22, 2013.

(51) Int. Cl. A61K 31/704; A61K 31/573

(52) U.S. Cl. CPC A61K 31/704; A61K 31/573

(57) ABSTRACT

Described herein are methods and compositions for intracellular delivery of therapeutic molecules. Disclosed herein are selective delivery conjugate comprising a targeting ligand conjugated to a selective delivery molecule (a) an acidic sequence (portion of A) which is effective to inhibit or prevent the uptake into cells or tissue retention, (b) a molecular transport or retention sequence (portion of B), and (c) a linker between portion of A and portion of B, and (d) at least one cargo moiety. Also, described are selective delivery molecules comprising a second linker comprising an intracellular cleavage site and optionally a self-immolative cleavage site.

### RELATED APPLICATION

This application is a continuation of U.S. application Ser. No. 14/786,402, filed Oct. 22, 2015, which is the National Phase entry of International Application No. PCT/US2014/035043, filed Apr. 22, 2014, which claims the benefit of and right of priority to U.S. Provisional Application No. 61/814,771, filed Apr. 22, 2013, each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Apr. 21, 2017, is named 39088711301_SL.txt and is 3,433,761 bytes in size.

### BACKGROUND OF THE INVENTION

Targeted delivery of therapeutic agents, such as cytotoxic agents, to tumor cells is desirable to avoid killing normal cells following systemic administration of such agents. Typical targeted drug delivery systems are composed of a cytotoxic agent conjugated to a tumor-specific antibody, forming an antibody-drug conjugate (ADC), also called an “immunoconjugate”. The tumor-specific antibody binds to a tumor biomarker (e.g. a tumor antigen) expressed on the surface of the tumor cells. When systemically administered, the ADC will selectively bind to tumor cells in the body, and thereby deliver the therapeutic agent intracellularly to the tumor cells, and not normal cells. The cytotoxic agent is not active when conjugated to the antibody, but becomes active upon being cleaved from the antibody intracellularly. of ADCs include gemtuzumab ozogamicin (Mylotarg), brentuximab vendotin (Adcetris), trastuzumab emtasine (Kadcyla).

### SUMMARY OF THE INVENTION

Described herein are compositions for the delivery of therapeutic agents.

Described herein, in certain embodiments, are selective delivery molecule conjugates comprising: (a) a selective delivery molecule of Formula I, having the structure:

A-X-B-[c_{B}-D_{B}] Formula I

wherein,

X is a cleavable linker;

A is a peptide with a sequence comprising 5 to 9 acidic amino acids:

B is a peptide with a sequence comprising 7 to 9 basic amino acids;

c_{B}, is 0-1 amino acid:

D_{B} is a therapeutic agent or an imaging agent;

wherein [c_{B}-D_{B}] is bound to any amino acid on B; and

(b) a carrier or targeting ligand, wherein the carrier or targeting ligand covalently bound to the selective delivery molecule. In some embodiments, the carrier or targeting ligand is covalently bound to any amino acid of A. In some embodiments, the carrier or targeting ligand is covalently bound to any amino acid of B. In some embodiments, the targeting ligand is an antibody. In some embodiments, the selective delivery molecule is covalently bound to any amino acid on the targeting antibody. In some embodiments, the selective delivery molecule is covalently bound to an amino acid in the Fc portion of the antibody. In some embodiments, the targeting ligand binds to a tumor antigen or tumor-specific receptor. In some embodiments, the targeting antibody binds to CD3, CD19, CD22, CD30, CD33, CD52, HER2 (ErB2), CD56 (NCAM), CS-125, Integrin, Cripto, glycoprotein NMB (osteoactivin), CD70, prostate specific membrane antigen (PSMA), or SLC44A4 (AGS-5). In some embodiments, the targeting antibody is gemtuzumab, inotuumab, trastuzumab, lorvotuzumab, imgn388, SAR3419, BiIB062, brentixumab, glembatumumab, SGN-75, PSMA ADC, ASG-5ME or mdx-1203. In some embodiments, the targeting ligand binds to a tumor antigen or tumor-specific receptor. In some embodiments, the targeting ligand is an integrin or a lectin. In some embodiments, the carrier is a polyethylene glycol (PEG) polymer. In some embodiments, A comprises a thiol reactive group. In some embodiments, the thiol reactive group is selected from among haloacetyls, maleimides, aziridines, acryloyls, arylating agents, vinylsulfones, pyridyl disulfides. TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In some embodiments, the therapeutic agent is a chemotherapeutic agent, a steroid, an immunotherapeutic agent, a targeted therapy, or an anti-inflammatory agent. In some embodiments, the therapeutic agent is a cytotoxin. In some embodiments, the therapeutic agent is doxorubicin, calicheamicin, maytansinoid, or auritstatin. In some embodiments, the therapeutic agent is cortisone. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} is selected from a naturally-occurring amino acid or a non-naturally-occurring amino acid. In some embodiments, c_{B} is selected from a D amino acid, a L amino acid, an α-amino acid, a β-amino acid, or a γ-amino acid. In some embodiments, c_{B} is selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} is selected from D-cysteine. D-glutamate, lysine, and para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10). RLQLKL (SEQ ID NO: 11), and RLQLK(Ac) (SEQ ID NO: 12). In some embodiments, the selective delivery molecule of Formula I is: SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

Described herein, in certain embodiments, are selective delivery molecule conjugates comprising: (a) a selective delivery molecule of Formula II, having the structure:

A-[c_{M}-M]-X-B-[c_{B}-D_{B}]Formula II

wherein,

X is a cleavable linker:

A is a peptide with a sequence comprising 5 to 9 acidic amino acids;

B is a peptide with a sequence comprising 7 to 9 basic amino acids;

c_{B} and c_{M} each independently comprise 0-1 amino acid;

M is a macromolecule;

D_{B} is a therapeutic agent or an imaging agent,

wherein [c_{M}-M] is bound to at any position on A or X, [c_{B}-D_{B}] is bound to any amino acid on B; and

(b) a carrier or targeting ligand, wherein the carrier or targeting ligand is covalently bound to the selective delivery molecule.

In some embodiments, the carrier or targeting ligand is covalently bound to any amino acid of A. In some embodiments, the carrier or targeting ligand is covalently bound to any amino acid of B. In some embodiments, the targeting ligand is an antibody. In some embodiments, the selective delivery molecule is covalently bound to any amino acid on the targeting antibody. In some embodiments, the selective delivery molecule is covalently bound to an amino acid in the Fc portion of the antibody. In some embodiments, the targeting antibody binds to a tumor antigen or a tumor antigen or tumor-specific receptor. In some embodiments, the targeting antibody binds to CD3, CD19, CD22, CD30, CD33, CD52, HER2 (ErB2), CD56 (NCAM), CS-125, Integrin, Cripto, glycoprotein NMB (osteoactivin), CD70, prostate specific membrane antigen (PSMA), or SLC44A4 (AGS-5). In some embodiments, the targeting antibody is gemtuzumab, inotuumab, trastuzumab, lorvotuzumab, imgn388, SAR3419, Bi1B062, brentixumab, glembatumumab. SGN-75, PSMA ADC, ASG-5ME or mdx-1203. In some embodiments, the targeting ligand binds to a tumor antigen or tumor-specific receptor. In some embodiments, the targeting ligand is an integrin or a lectin. In some embodiments, A comprises a thiol reactive group. In some embodiments, the thiol reactive group is selected from among haloacetyls, maleimides, aziridines, acryloyls, arylating agents, vinylsulfones, pyridyl disulfides. TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In some embodiments, the therapeutic agent is a chemotherapeutic agent, a steroid, an immunotherapeutic agent, a targeted therapy, or an anti-inflammatory agent. In some embodiments, the therapeutic agent is a cytotoxin. In some embodiments, the therapeutic agent is doxorubicin, calicheamicin, maytansinoid, or auritstatin. In some embodiments, the therapeutic agent is cortisone. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} and c_{M} are each independently selected from a naturally-occurring amino acid or a non-naturally-occurring amino acid. In some embodiments, c_{B} and c_{M} are each independently selected from a D amino acid, a L amino acid, an α-amino acid, a β-amino acid, or a γ-amino acid. In some embodiments, c_{B} and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} and c_{M} are each independently selected from D-cysteine, D-glutamate, lysine, and para-4-acetyl L-phenylalanine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments. X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments. M is selected from a protein, a natural polymer, a synthetic polymer, or a dendrimer. In some embodiments, M is selected from dextran, a polyethylene glycol (PEG) polymer, albumin, or a combination thereof. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, the selective delivery molecule of Formula II is: SDM-101, SDM-102, SDM-103, SDM-104, SDM-105, SDM-106, SDM-107, SDM-108, SDM-109, SDM-110, SDM-111, SDM-12, SDM-13, SDM-14, SDM-15, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

Described herein, in certain embodiments, are selective delivery molecule conjugates comprising: (a) a selective delivery molecule of Formula V, having the structure:

A-[c_{M}-M]-X-B-Y-[c_{B}-D_{B}]Formula V

wherein,

X is a cleavable linker:

Y is a cleavable linker;

A is a peptide with a sequence comprising 5 to 9 acidic amino acids:

B is a peptide with a sequence comprising 7 to 9 basic amino acids:

c_{B} and c_{M} each independently comprise 0-1 amino acid;

M is a macromolecule;

D_{B} is a therapeutic agent or an imaging agent,

wherein [c_{M}-M] is bound to at any position on A or X, and [c_{B}-D_{B}] is bound to any amino acid on B; and

(b) a carrier or targeting ligand, wherein the carrier or targeting ligand covalently bound to the selective delivery molecule.

In some embodiments, the carrier or targeting ligand is covalently bound to any amino acid of A. In some embodiments, the carrier or targeting ligand is covalently bound to any amino acid of B. In some embodiments, the targeting ligand is an antibody. In some embodiments, the selective delivery molecule is covalently bound to any amino acid on the targeting antibody. In some embodiments, the selective delivery molecule is covalently bound to an amino acid in the Fc portion of the antibody. In some embodiments, the targeting antibody binds to a tumor antigen or a tumor antigen or tumor-specific receptor. In some embodiments, the targeting antibody binds to CD3, CD19, CD22, CD30, CD33, CD52, HER2 (ErB2), CD56 (NCAM), CS-125, Integrin, Cripto, glycoprotein NMB (osteoactivin), CD70, prostate specific membrane antigen (PSMA), or SLC44A4 (AGS-5). In some embodiments, the targeting antibody is gemtuzumab, inotuumab, trastuzumab, lorvotuzumab, imgn388, SAR3419, Bi1B062, brentixumab, glembatumumab, SGN-75, PSMA ADC, ASG-5ME or mdx-1203. In some embodiments, the targeting ligand binds to a tumor antigen or tumor-specific receptor. In some embodiments, the targeting ligand is an integrin or a lectin. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In some embodiments, the therapeutic agent is a chemotherapeutic agent, a steroid, an immunotherapeutic agent, a targeted therapy, or an anti-inflammatory agent. In some embodiments, the therapeutic agent is a cytotoxin. In some embodiments, the therapeutic agent is doxorubicin, calicheamicin, maytansinoid, or auritstatin. In some embodiments, the therapeutic agent is cortisone. In some embodiments. A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} and c_{M} are each independently selected from a naturally-occurring amino acid or a non-naturally-occurring amino acid. In some embodiments, c_{B} and c_{M} are each independently selected from a D amino acid, a L amino acid, an α-amino acid, a β-amino acid, or a γ-amino acid. In some embodiments, c_{B} and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} and c_{M} are each independently selected from D-cysteine, D-glutamate, lysine, and para-4-acetyl L-phenylalanine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10). RLQLKL (SEQ ID NO: 11), and RLQLK(Ac) (SEQ ID NO: 12). In some embodiments, Y is cleavable by a protease. In some embodiments, Y is cleavable by an intracellular protease. In some embodiments, Y is cleavable by a lysosomal protease. In some embodiments, Y is cleavable by Cathepsin B. In some embodiments, Y comprises a self-immolative spacer. In some embodiments, Y comprises a PABC spacer or a derivative thereof. In some embodiments, M is selected from a protein, a natural polymer, a synthetic polymer, or a dendrimer. In some embodiments, M is selected from dextran, a polyethylene glycol (PEG) polymer, albumin, or a combination thereof. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 100 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, the selective delivery molecule of Formula V is: SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

Described herein, in certain embodiments, are selective delivery molecules of Formula V, having the structure:

A-[c_{M}-M]-X-B-Y-[c_{B}-D_{B}]Formula V

wherein,

X is a cleavable linker;

Y is a cleavable linker;

A is a peptide with a sequence comprising 5 to 9 acidic amino acids;

B is a peptide with a sequence comprising 7 to 9 basic amino acids;

c_{B} and c_{M} each independently comprise 0-1 amino acid;

M is a macromolecule:

D_{B} is a therapeutic agent or an imaging agent; and

wherein [c_{M}-M] is bound to at any position of A or X, and [c_{B}-D_{B}] is bound to any amino acid of B. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} and c_{M} are each independently selected from a naturally-occurring amino acid or a non-naturally-occurring amino acid. In some embodiments, c_{B} and c_{M} are each independently selected from a D amino acid, a L amino acid, an α-amino acid, a β-amino acid, or a γ-amino acid. In some embodiments, c_{B} and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} and c_{M} are each independently selected from D-cysteine, D-glutamate, lysine, and para-4-acetyl L-phenylalanine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments, Y is cleavable by a protease. In some embodiments, Y is cleavable by an intracellular protease. In some embodiments, Y is cleavable by a lysosomal protease. In some embodiments. Y is cleavable by Cathepsin B. In some embodiments, Y comprises a self-immolative spacer. In some embodiments, Y comprises a PABC spacer or a derivative thereof. In some embodiments, the therapeutic agent is a chemotherapeutic agent, a steroid, an immunotherapeutic agent, a targeted therapy, or an anti-inflammatory agent. In some embodiments, the therapeutic agent is a cytotoxin. In some embodiments, the therapeutic agent is doxorubicin, calicheamicin, maytansinoid, or auritstatin. In some embodiments, the therapeutic agent is cortisone. In some embodiments, M is selected from a protein, a natural polymer, a synthetic polymer, or a dendrimer. In some embodiments. M is selected from dextran, a polyethylene glycol (PEG) polymer, albumin, or a combination thereof. In some embodiments, M is selected from a PEG polymer having an average molecular weight of approximately 0.5 kDa (PEG 0.5 kDa), approximately 10 kDa (PEG 10 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, molecule is SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153. In some embodiments, the selective delivery molecule is covalently bound to a carrier or targeting ligand. In some embodiments, the carrier or targeting ligand is covalently bound to any amino acid of A. In some embodiments, the carrier or targeting ligand is covalently bound to any amino acid of B. In some embodiments, the targeting ligand is an antibody. In some embodiments, the selective delivery molecule is covalently bound to any amino acid on the targeting antibody. In some embodiments, the selective delivery molecule is covalently bound to an amino acid in the Fc portion of the antibody. In some embodiments, the targeting antibody binds to a tumor antigen or a tumor antigen or tumor-specific receptor. In some embodiments, the targeting antibody binds to CD3, CD19, CD22, CD30, CD33, CD52, HER2 (ErB2), CD56 (NCAM), CS-125, Integrin, Cripto, glycoprotein NMB (osteoactivin), CD70, prostate specific membrane antigen (PSMA), or SLC44A4 (AGS-5). In some embodiments, the targeting antibody is gemtuzumab, inotuumab, trastuzumab, lorvotuzumab, imgn388, SAR3419, Bi1B062, brentixumab, glembatumumab, SGN-75, PSMA ADC, ASG-5ME or mdx-1203. In some embodiments, the targeting ligand binds to a tumor antigen or tumor-specific receptor. In some embodiments, the targeting ligand is an integrin or a lectin.

Described herein, in certain embodiments, are selective delivery molecule conjugates comprising: a carrier or targeting ligand; and any selective delivery molecule provided herein. In some embodiments, the carrier or targeting ligand is covalently bound to any amino acid of A. In some embodiments, the carrier or targeting ligand is covalently bound to any amino acid of B. In some embodiments, the targeting ligand is an antibody. In some embodiments, the selective delivery molecule is covalently bound to any amino acid on the targeting antibody. In some embodiments, the selective delivery molecule is covalently bound to an amino acid in the Fc portion of the antibody. In some embodiments, the targeting antibody binds to a tumor antigen or a tumor antigen or tumor-specific receptor. In some embodiments, the targeting antibody binds to CD3, CD19, CD22, CD30, CD33, CD52, HER2 (ErB2). CD56 (NCAM), CS-125, Integrin, Cripto, glycoprotein NMB (osteoactivin). CD70, prostate specific membrane antigen (PSMA), or SLC44A4 (AGS-5). In some embodiments, the targeting antibody is gemtuzumab, inotuumab, trastuzumab, lorvotuzumab, imgn388, SAR3419, Bi1B062, brentixumab, glembatumumab, SGN-75, PSMA ADC. ASG-5ME or mdx-1203. In some embodiments, the targeting ligand binds to a tumor antigen or tumor-specific receptor. In some embodiments, the targeting ligand is an integrin or a lectin. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo.

Described herein, in certain embodiments, are selective delivery molecule according to SDM-101, SDM-102, SDM-103, SDM-104, SDM-105, SDM-106, SDM-107, SDM-108, SDM-109, SDM-10, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, or SDM-152

Described herein, in certain embodiments, are pharmaceutical compositions comprising a selective delivery molecule conjugate provided herein and one or more pharmaceutically acceptable carriers, glidants, diluents, or excipients.

Described herein, in certain embodiments, are methods for treating cancer in a subject in need thereof, comprising administering to a subject having cancer a therapeutically effective amount of a selective delivery molecule conjugate provided herein, thereby treating the cancer.

In some embodiments, the cancer is a breast cancer, colorectal cancer, ovarian cancer, lung cancer, esophageal cancer, pancreatic cancer, gastro-intestinal cancer, squamous cell carcinoma, prostate cancer, melanoma, or thyroid cancer. In some embodiments, the therapeutic agent is a chemotherapeutic agent. In some embodiments, the therapeutic agent is a cytotoxin. In some embodiments, the methods further comprise administering an additional anti-cancer agent.

Described herein, in certain embodiments, are methods for treatment of inflammation in a subject in need thereof, comprising administering to the subject a therapeutically effective amount of a selective delivery molecule conjugate provided herein, thereby treating the inflammation. In some embodiments, the inflammation is acute inflammation or chronic inflammation. In some embodiments, the inflammation is associated with rheumatoid arthritis, osteoarthritis, inflammatory bowel disease, Crohn's disease, ulcerative colitis, sepsis, erythema nodosum leprosum, multiple sclerosis, psoriasis, systemic lupus erythematosis, type I diabetes, atherosclerosis, encephalomyelitis, Alzheimer's disease, stroke, traumatic brain injury. Parkinson's disease or septic shock. In some embodiments, the therapeutic agent is an anti-inflammatory agent. In some embodiments, the therapeutic agent is a steroid.

Described herein, in certain embodiments, are methods for treatment of an autoimmune disease in a subject in need thereof, comprising administering to the subject a therapeutically effective amount of a selective delivery molecule conjugate provided herein, thereby treating the autoimmune disease. In some embodiments, the autoimmune disease is Celiac disease, diabetes mellitus type 1, Sarcoidosis, systemic lupus erythematosus (SLE), Sjögren's syndrome, Churg-Strauss Syndrome, Hashimoto's thyroiditis, Graves' disease, idiopathic thrombocytopenic purpura, Addison's Disease, rheumatoid arthritis (RA), Polymyositis (PM), or Dermatomyositis (DM).

Described herein, in certain embodiments, are methods for delivering a therapeutic agent or an imaging agent to a cancer cell in a subject, comprising administering to the subject having cancer a selective delivery molecule conjugate provided herein or a selective delivery molecule provided herein, thereby delivering a therapeutic agent or an imaging agent to a cancer cell. In some embodiments, the methods further comprise imaging the cancer.

Described herein, in certain embodiments, are methods for delivering a therapeutic agent or an imaging agent to a site of inflammation in a subject, comprising administering to the subject having inflammation a selective delivery molecule conjugate provided herein or a selective delivery molecule provided herein, thereby delivering a therapeutic agent or an imaging agent to the site of inflammation. In some embodiments, the methods further comprise imaging the site of inflammation.

### BRIEF DESCRIPTION OF THE DRAWINGS

FIG. 1: Cleavage of SDM-145 by hMMP-9. A) HPLC chromatogram of SDM-145; B) HPLC chromatogram of the reaction mixture after incubation at 37° C. for 17 h. LC-MS confirmed that the molecular weight of the peak at ˜9.4 min was consistent with the fragment generated by hMMP-9 cleavage at the expected cleavage site. The fragment's chemical structure was shown in the chromatogram.

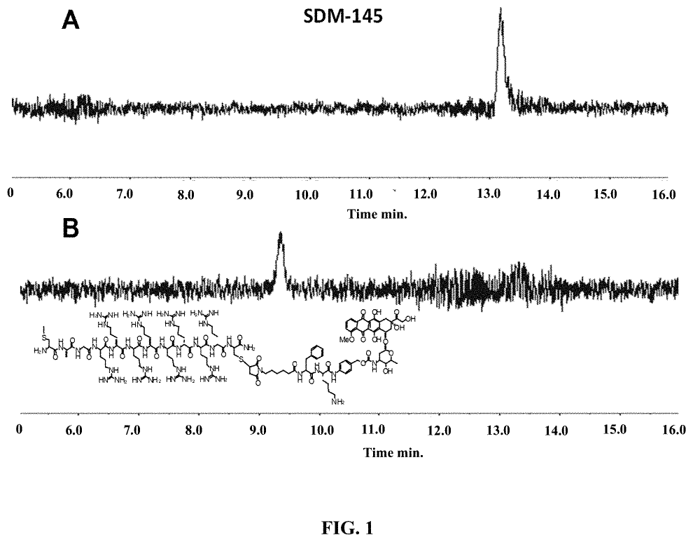

FIG. 2: Cleavage of SDM-145 by Cathepsin B. A) HPLC chromatogram of conjugate SDM-145; B) HPLC chromatogram of the reaction mixture after incubation at 37° C. for 17 h. LC-MS confirmed that the molecular weight of the peak at 9.0 min was consistent with the freed doxorubicin.

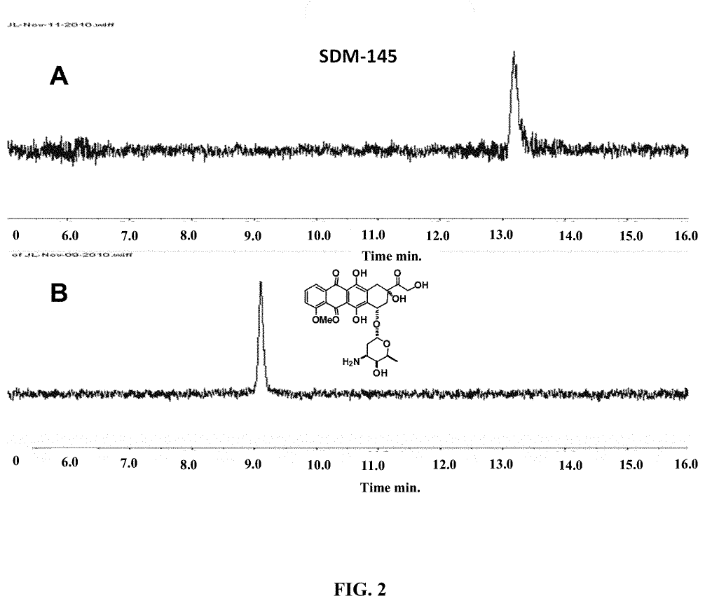

FIG. 3 illustrates schematics of exemplary protease activated antibody conjugates and protease cleavage steps.

FIG. 4 illustrates a schematic of exemplary dual protease activated drug delivery conjugate and protease cleavage steps.

FIG. 5 illustrates a higher resolution schematic of exemplary dual protease activated drug delivery conjugate.

FIG. 6 illustrates an exemplary scheme for protease cleavage and uptake of a dual protease activated drug delivery conjugate: (a) extracellular proteases (e.g. matrix metalloproteinases) cleave conjugate near target cells, (b) cell penetrating peptide (CPP)-drug portion enters target cells, (c) lysosomal proteases (e.g. Cathepsin B) cleave linker, and (d) active therapeutic cargo (e.g. Doxorubicin) is released.

FIG. 7 illustrates a schematic of exemplary thiol-reactive drug delivery conjugates. The SDMs react efficiently with albumin Cys(34)-SH in circulation albumin after injected into blood stream. Albumin-SDM conjugates have improved pharmacokinetic profiles and efficient targeted cargo delivery.

FIG. 8 illustrates MALDI-TOF Spectrum of SDM-147.

FIG. 9 illustrates therapeutic activity of SDM-147 in a 4T1 breast cancer mouse model.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information. Generally, the procedures for cell culture, cell infection, antibody production and molecular biology methods are methods commonly used in the art. Such standard techniques can be found, for example, in reference manual, such as, for example. Sambrook et al. (2000) and Ausubel et al. (1994).

As used herein, the singular forms “a.” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”. “includes”, and “included”) is not limiting.

As used herein, ranges and amounts can be expressed as “about” a particular value or range. About also includes the exact amount. Hence “about 40 mg” means “about 40 mg” and also “40 mg.” Generally, the term “about” includes an amount that would be expected to be within experimental error.

The terms “individual,” “patient,” or “subject” are used interchangeably. As used herein, they mean any mammal (i.e. species of any orders, families, and genus within the taxonomic classification animalia chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range: e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “C1-C4 alkyl” or similar designations. By way of example only, “C1-C4 alkyl” indicates that there are one to four carbon atoms in the alkyl chain, i.e., the alkyl chain is selected from: methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In some embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. alkyls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In some embodiments, the ring is a cycloalkane. In some embodiments, the ring is a cycloalkene.

In some embodiments, the ring is an aromatic ring. The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In some embodiments, the ring is a heterocycle. The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

In some embodiments, the ring is fused. The term “fused” refers to structures in which two or more rings share one or more bonds. In some embodiments, the ring is a dimer. In some embodiments, the ring is a trimer. In some embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

In some embodiments, the linker is substituted. The term “optionally substituted” or “substituted” means that the referenced group may be substituted with one or more additional group(s) individually and independently selected from C₁-C₆alkyl. C₃-C₅cycloalkyl, aryl, heteroaryl, C₂-C₆heteroalicyclic, hydroxy, C₁-C₆alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₆acyl, C₁-C₈acyloxy, nitro, C₁-C₅haloalkyl, C₁-C₈fluoroalkyl, and amino, including C₁-C₆alkylamino, and the protected derivatives thereof. By way of example, an optional substituents may be LsRs, wherein each Ls is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each Rs is independently selected from H, (C₁-C₄alkyl). (C₃-C₈cycloalkyl), heteroaryl, aryl, and C₁-C₆heteroalkyl. Optionally substituted non-aromatic groups may be substituted with one or more oxo (═O). The protecting groups that may form the protective derivatives of the above substituents are known to those of skill in the art.

### Overview

Selective delivery molecules (SDMs) allow the targeted delivery of therapeutic agents and/or imaging agents to specific cells and/or tissues. In certain embodiments, selective delivery molecules comprise (a) an acidic sequence (portion of A) which is effective to inhibit or prevent uptake of the molecule into cells or tissue retention, (b) a molecular transport or retention sequence (portion of B) (e.g., a cell penetrating peptide (CPP), (c) a cleavable linker X located between portion of A and portion of B, (d) at least one cargo moiety (portion D) bound to portion of B, and optionally (e) a macromolecular carrier bound to portion of A. In some embodiments, cleavage of the X linker allows the separation of portion of A from portion of B, thereby promoting the uptake or retention of portion of B and the attached cargo into cells or tissue retention. In some embodiments, the therapeutic cargo is a chemotherapeutic agent. In some embodiments, the therapeutic cargo is a cytotoxin.

As described herein, in certain instances, conjugating a selective delivery molecule disclosed herein to a targeting ligand, such as an antibody, allows the SDM to be targeted to specific cells having a specific cell surface marker. Similarly, as described herein, in certain instances, the cell specific delivery of an existing targeting ligand-drug conjugate (e.g., an antibody-drug conjugate) is improved by attachment of a selective delivery molecule described herein to the targeting ligand. In some embodiments, the targeting antibody binds to a tumor antigen or a receptor that is upregulated in tumor cells. Accordingly, provided herein are improved antibody-drug conjugates for delivery of therapeutic agents to target cells and tissues, such as cancer cells and other diseased cells.

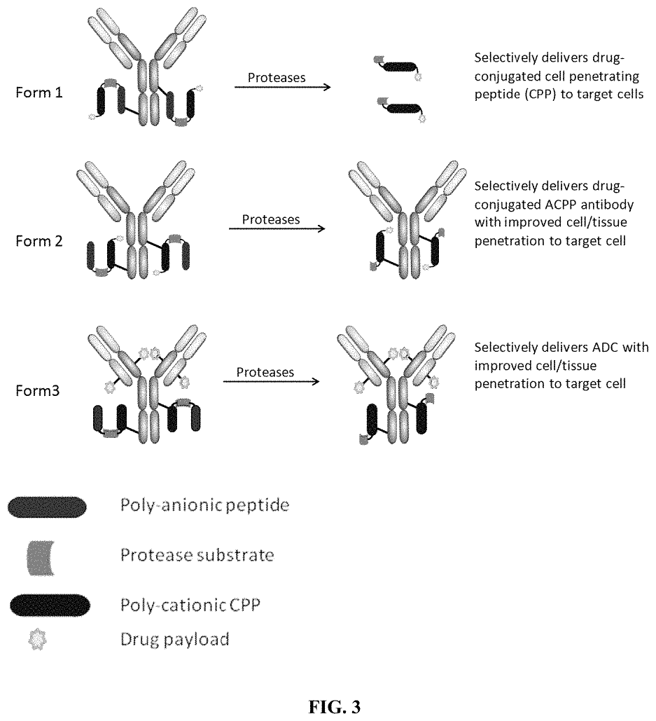

In some embodiments, the targeting ligand-conjugated SDMs described herein provide significant advantages over existing targeting ligand-drug conjugates (e.g., antibody-drug conjugates). In some embodiments, the targeting ligand-conjugated SDMs described herein provide improved tumor penetration and retention over existing targeting ligand-drug conjugates. In some embodiments, the targeting ligand-conjugated SDMs described herein provide dual targeting specificity. In some embodiments, the dual targeting mechanism comprises 1) ligand (e.g., antibody) targeting of cell specific markers on diseased cells and 2) pathological protease activity targeting of increased extracellular protease activity at physiological location of the diseased cell. In some embodiments, the targeting ligand-conjugated SDMs described herein provide different options for conjugating a variety of therapeutic cargo molecules for delivery to the diseased cell. For example, in certain embodiments, a variety of configurations is available for the attachment of the therapeutic cargo to the SDM or the ligand (e.g., antibody) itself. FIG. 3 illustrates exemplary configurations of targeting ligand-conjugated SDMs, where the targeting ligand is an antibody. In some embodiments, the therapeutic cargo is internalized with the targeting ligand. In some embodiments, the therapeutic cargo is released from the targeting ligand-conjugated SDM prior to uptake by the cell. In addition, the modular design of the targeting ligand-conjugated SDMs described herein enables easy modification of the conjugates to change protease recognition sites and therapeutic cargo molecules.

In some embodiments, increased retention and penetration of the cells achieved by the SDM conjugates provided herein increase the efficacy of the therapeutic cargo. In some embodiments, a lower dosage of the therapeutic cargo is employed compared to existing targeting ligand-drug conjugates that lack an SDM. In some embodiments, a less toxic therapeutic cargo is needed to treat the target cell. In some embodiments, increased retention and penetration of the cells achieved by the conjugates provided herein allows for less toxic therapeutic cargo molecules to be employed compared to existing targeting ligand-drug conjugates that lack an SDM.

Described herein, in certain embodiments are SDMs that provide single protease targeting. For example, in some embodiments, cleavage of the X linker located between portion of A and portion of B of the SDM by a protease located near the target cell allows the separation of portion of A from portion of B, thereby promoting the uptake or retention of portion of B and the attached cargo into cells or tissue retention. In some embodiments, the protease exhibits higher expression in the extracellular area surrounding the target cell (e.g., a cancer cell) as compared to a non-target cell (e.g., non-diseased/non-cancerous cell). In some embodiments, the protease is a matrix metalloproteinase (MMP). In some embodiments, the therapeutic cargo is a chemotherapeutic agent. In some embodiments, the therapeutic cargo is a cytotoxin. In some embodiments, the therapeutic cargo is doxorubicin, calicheamicin, maytansinoid, or auritstatin. In some embodiments, the therapeutic cargo is an anti-inflammatory agent. In some embodiments, the therapeutic cargo is a steroid. In some embodiments, the therapeutic cargo is cortisone or a derivative thereof. In some embodiments, the SDM is conjugated to a targeting ligand.

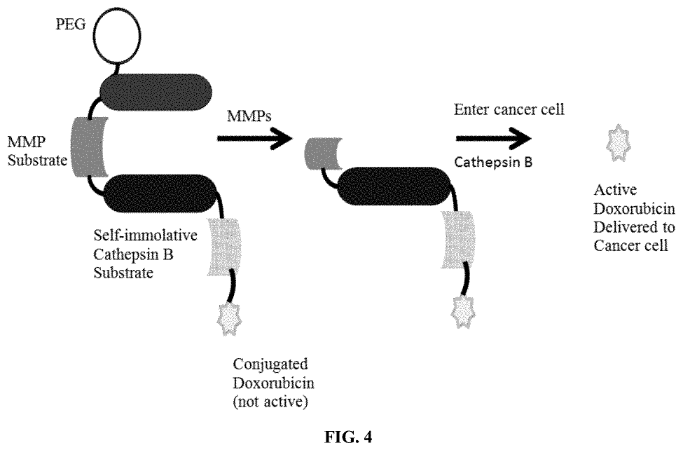

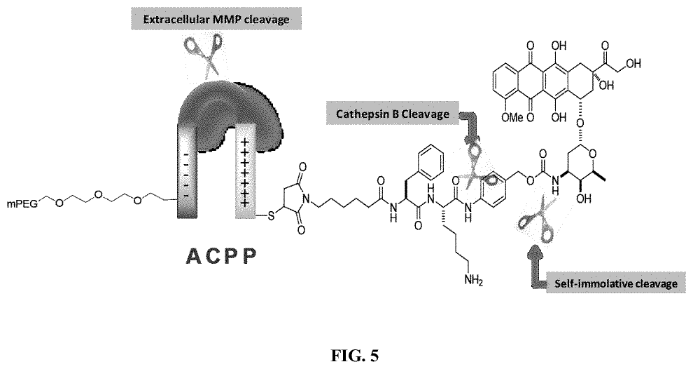

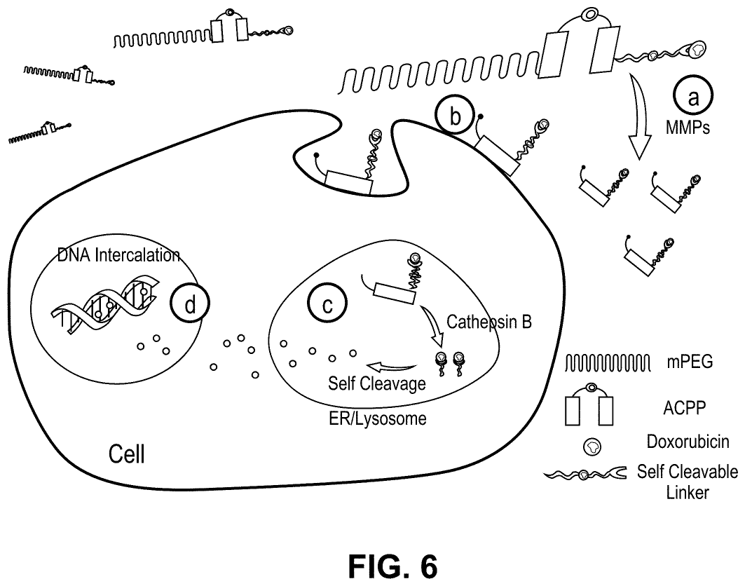

Described herein, in certain embodiments are SDMs that provide double protease targeting. For example, in some embodiments, the SDM comprises an X linker located between portion of A and portion of B and a second linker Y located between portion of B and the therapeutic cargo molecule (portion D). In some embodiments, the X linker comprises an extracellular protease cleavage site and the Y linker comprises an intracellular protease cleavage site. In some embodiments, cleavage of the X linker located between portion of A and portion of B of the selective delivery molecule by a protease located near the target cell allows the separation of portion of A from portion of B, thereby promoting the uptake or retention of portion of B and the attached cargo into cells or tissue retention, and then cleavage of the Y linker by an intracellular protease allows the therapeutic cargo to be released into the cell after uptake. In some embodiments, such configurations allow therapeutic cargoes to be maintained in an inactive state until the therapeutic cargoes are taken up by the cell following cleavage of the X linker. In some embodiments, the extracellular protease that cleaves the X linker exhibits a higher expression in the extracellular area surrounding the target cell (e.g., a cancer cell) as compared to a non-target cell (e.g., non-diseased/non-cancerous cell). In some embodiments, the extracellular protease is a matrix metalloproteinase (MMP). In some embodiments, the intracellular protease that cleaves the Y linker is a lysosomal protease. In some embodiments, the protease is one that is activated at low pH. In some embodiments, the protease is one that is activated at low pH and is expressed in an endosome. In some embodiments, the protease is a cathepsin. In some embodiments, the cathepsin is cathepsin B or cathepsin D. In some embodiments, the cathepsin is cathepsin B. In some embodiments, the Y linker additionally comprises a self-immolative cleavage site located between the intracellular protease cleavage site and the attached therapeutic cargo. In some embodiments, the self-immolative cleavage site is a PABC (p-aminobenzylcarbonyl) spacer and analogs thereof. In some embodiments, the self-immolative cleavage site is a thiazole containing linker. In some embodiments, the selective delivery molecule comprising an X linker located between portion of A and portion of B and a second linker Y located between portion of B and the therapeutic cargo molecule (portion D) is conjugated to a targeting ligand, such as a targeting antibody. In some embodiments, the targeting ligand binds to a tumor antigen or a receptor that is upregulated in tumor cells. In some embodiments, the SDM is conjugated to a targeting ligand. FIGS. 4 and 5 illustrate schematics of exemplary dual protease drug delivery conjugates. FIG. 6 illustrates delivery of an antibody-SDM conjugate with dual protease targeting.

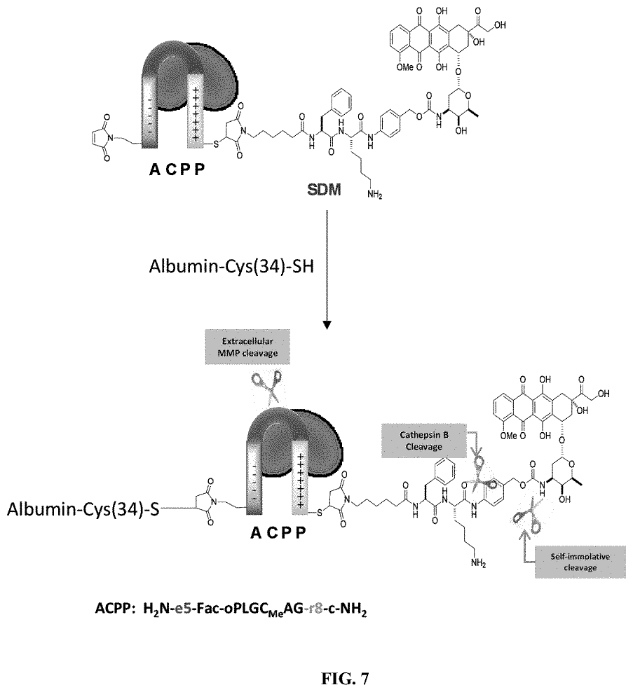

In some embodiments, the SDMs provided herein are conjugated to albumin and/or contain a free thiol reactive group for interacting with albumin in vivo. Albumin is a carrier for tumor targeting because it accumulates in solid tumors due to the pathophysiology of tumor tissue, characterized by a high metabolic turnover, angiogenesis, hypervasculature, a defective vascular architecture and an impaired lymphatic drainage. The unique free sulfhydryl group (Cys-34) of albumin, which is not present in the majority of circulating serum proteins, is accessible for selective modifications. Albumin-drug conjugates show improved the pharmacokinetic profiles. However, albumin conjugates have limited tumor penetration and distribution due to their big molecular size and the tumor tissue's microenvironment, such as increased interstitial fluid pressure and dense extracellular matrix. In some embodiments, thiol-reactive SDMs provided herein form albumin conjugates in vivo. In some embodiments, the conjugates increase the drug's tumor penetration. In some embodiments, the conjugates increase improve drug's distribution and activity. In some embodiments, after injected into blood stream, thiol-reactive SDMs react with the free Cys34 thiol of the circulating albumin. The albumin-SDM conjugate is then transported and accumulated in the tumor tissues. The up-regulated MMPs in tumor tissues cleave the MMPs sensitive linker in ACPP part and release the poly-Arg-drug fragment. As poly-Arg has excellent cell penetrating capability, the poly-Arg-drug fragment is able to efficiently bind to the tumor cell and get internalized. After internalization, drug is regenerated after an enzymatic cleavage by intracellular proteases, such as Cathepsin B in the lysosome. In some embodiments, the thiol reactive group of the SDM is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. FIG. 7 illustrates exemplary schematics of exemplary thiol-reactive SDMs.

### Conjugated Selective Delivery Molecules (SDMs)

Disclosed herein, in certain embodiments, and carrier-conjugated SDMs. In some embodiments, a carrier modulates plasma half-life of a selective delivery molecule disclosed herein. In some embodiments, a carrier modulates solubility of a selective delivery molecule disclosed herein. In some embodiments, a carrier modulates bio-distribution of a selective delivery molecule disclosed herein.

In some embodiments, a carrier decreases uptake of a selective delivery molecule by non-target cells or tissues. In some embodiments, a carrier decreases uptake of a selective delivery molecule into cartilage. In some embodiments, a carrier decreases uptake of a selective delivery molecule into joints relative to target tissue.

In some embodiments, a carrier increases uptake of a selective delivery molecule by target cells or tissues. In some embodiments, a carrier decreases uptake of a selective delivery molecule into the liver relative to target tissue. In some embodiments, a carrier decreases uptake of a selective delivery molecule into kidneys. In some embodiments, a carrier enhances uptake into cancer tissue. In some embodiments, a carrier enhances uptake into lymphatic channels and/or lymph nodes.

In some embodiments, a carrier increases plasma half-life by reducing glomerular filtration. In some embodiments, a carrier modulates plasma half-life by increasing or decreases metabolism or protease degradation. In some embodiments, a carrier increases tumor uptake due to enhanced permeability and retention (EPR) of tumor vasculature. In some embodiments, a carrier increases the aqueous solubility of selective delivery molecule.

In some embodiments, any carrier is independently directly or indirectly (e.g., via c_{M}) bound to A, B, or X. In some embodiments, any carrier is independently bound to A at the n-terminal poly glutamate. In some embodiments, any carrier is independently bound to A (or, the n-terminal poly glutamate) by a covalent linkage. In some embodiments, any carrier is independently bound to B at the c-terminal polyarginine. In some embodiments, any carrier is independently bound to B (or, the c-terminal polyarginine) by a covalent linkage. In some embodiments, any carrier is independently directly or indirectly bound to linkers between X and A, X and B. B and C/N terminus, and A and C/N terminus. In some embodiments, the covalent linkage comprises an ether bond, thioether bond, amine bond, amide bond, oxime bond, carbon-carbon bond, carbon-nitrogen bond, carbon-oxygen bond, or carbon-sulfur bond.

In some embodiments, carrier is selected from a macromolecule such as a protein, a synthetic or natural polymer, or a dendrimer. In some embodiments, carrier is selected from dextran, a PEG polymer (e.g., a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)), albumin, or a combination thereof. In some embodiments, carrier is a PEG polymer.

In some embodiments, the size of carrier is between about 500 kDa and about 70 kDa.

In some embodiments, the selective delivery molecule is conjugated to albumin. In certain instances, albumin is excluded from the glomerular filtrate under normal physiological conditions. In some embodiments, the selective delivery molecule comprises a reactive group such as maleimide that can form a covalent conjugate with albumin. A selective delivery molecule comprising albumin results in enhanced accumulation of cleaved selective delivery molecules in tumors in a cleavage dependent manner. In some embodiments, albumin conjugates have good pharmacokinetic properties.

In some embodiments, the selective delivery molecule is conjugated to PEG polymers.

In some embodiments, the selective delivery molecule is conjugated to PEG polymers having an average molecular weight of approximately 0.5 kDa (PEG 0.5 kDa). In some embodiments, the selective delivery molecule is conjugated to PEG polymers having an average molecular weight of approximately 1 kDa (PEG 1 kDa). In some embodiments, the selective delivery molecule is conjugated to PEG polymers having an average molecular weight of approximately 2 kDa (PEG 2 kDa). In some embodiments, the selective delivery molecule is conjugated to PEG polymers having an average molecular weight of approximately 3 kDa (PEG 3 kDa). In some embodiments, the selective delivery molecule is conjugated to PEG polymers having an average molecular weight of approximately 4 kDa (PEG 4 kDa). In some embodiments, the selective delivery molecule is conjugated to PEG polymers having an average molecular weight of approximately 5 kDa (PEG 5 kDa). In some embodiments, the selective delivery molecule is conjugated to PEG polymers having an average molecular weight of approximately 10 kDa (PEG 10 kDa). In some embodiments, the selective delivery molecule is conjugated PEG polymers having an average molecular weight of approximately 12 kDa (PEG 12 kDa). In some embodiments, the selective delivery molecule is conjugated to PEG polymers having an average molecular weight of approximately 15 kDa (PEG 15 kDa). In some embodiments, selective delivery molecule is conjugated to PEG polymers having an average molecular weight of approximately 20 kDa (PEG 20 kDa). In some embodiments, selective delivery molecule is conjugated to PEG polymers having an average molecular weight of approximately 30 kDa (PEG 30 kDa). In some embodiments, selective delivery molecules conjugated to PEG30 kDa had a longer half-life as compared to free peptides. In some embodiments, selective delivery molecules are conjugated to PEG polymers having an average molecular weight of between about 20 to about 40 kDa which have hepatic and renal clearance.

In some embodiments, the selective delivery molecule is conjugated to a dextran. In some embodiments, the selective delivery molecule is conjugated to a dextran having an average molecular weight of approximately 70 kDa. In some embodiments, dextran conjugates, being a mixture of molecular weights, are difficult to synthesize and purify reproducibly.

In some embodiments, the selective delivery molecule is conjugated to streptavidin.

In some embodiments, the selective delivery molecule is conjugated to a fifth generation PAMAM dendrimer.

In some embodiments, a carrier is capped. In some embodiments, capping a carrier improves the pharmacokinetics and reduces cytotoxicity of a carrier by adding hydrophilicity. In some embodiments, the cap is selected from: Acetyl, succinyl, 3-hydroxypropionyl, 2-sulfobenzoyl, glycidyl, PEG-2, PEG-4, PEG-8 and PEG-12.

Disclosed herein, in certain embodiments, are targeting ligand-conjugated SDMs. In some embodiments, a therapeutic cargo and an SDM are conjugated to a targeting ligand. In some embodiments, the SDM comprises a therapeutic cargo and the SDM comprising the therapeutic cargo is conjugated to a targeting ligand. Thus, in some embodiments, the therapeutic cargo and the SDM are conjugated to the same site on the targeting ligand. In some embodiments, the therapeutic cargo is first conjugated to an SDM, and then the SDM comprising the therapeutic cargo is attached to the targeting ligand.

In some embodiments, the therapeutic cargo and the SDM are conjugated to two different sites on the targeting ligand. In some embodiments, the SDM is conjugated to an existing ligand-drug conjugate. In some embodiments, an SDM is conjugated to a targeting ligand, and then a therapeutic cargo is conjugated to the targeting ligand-SDM conjugate. In some embodiments, a therapeutic cargo is conjugated to a targeting ligand, and then an SDM is conjugated to the targeting ligand-therapeutic cargo conjugate.

In some embodiments, the targeting ligand is conjugated to the acidic sequence (portion of A) of an SDM.

In some embodiments, the targeting ligand is conjugated to molecular transport or retention sequence (portion of B) of an SDM.

In some embodiments, any of a variety of known methods for conjugation of molecules to polypeptides such as targeting ligands are employed for the conjugation of the therapeutic cargo and/or SDMs provided herein.

Disclosed herein, in certain embodiments, are antibody-conjugated SDMs. In some embodiments, a therapeutic cargo and an SDM are conjugated to a targeting antibody. In some embodiments, the SDM comprises a therapeutic cargo and the SDM comprising the therapeutic cargo is conjugated to a targeting antibody. Thus, in some embodiments, the therapeutic cargo and the SDM are conjugated to the same site on the targeting antibody (e.g., Form 1 and Form 2 of FIG. 3). In some embodiments, the therapeutic cargo is first conjugated to an SDM, and then the SDM comprising the therapeutic cargo is attached to the targeting antibody.

In some embodiments, the therapeutic cargo and the SDM are conjugated to two different sites on the targeting antibody (e.g., Form 3 of FIG. 3). In some embodiments, the SDM is conjugated to an existing antibody-drug conjugate. In some embodiments, an SDM is conjugated to a targeting antibody, and then a therapeutic cargo is conjugated to the antibody-SDM conjugate. In some embodiments, a therapeutic cargo is conjugated to a targeting antibody, and then an SDM is conjugated to the antibody-therapeutic cargo conjugate.

In some embodiments, the targeting antibody is conjugated to the acidic sequence (portion of A) of an SDM (Form 1, FIG. 3).

In some embodiments, the targeting antibody is conjugated to molecular transport or retention sequence (portion of B) of an SDM (Form 2 and Form 3. FIG. 3).

In some embodiments, any of a variety of known methods for conjugation of molecules to antibodies are employed for the conjugation of the therapeutic cargo and/or SDMs provided herein.

### Targeting Ligands

In some embodiments, the targeting ligand is a molecule that binds to a cell surface molecule expressed on the surface of a target cell. In some embodiments, the targeting ligand binds to a receptor expressed on the surface of a target cell. In some embodiments, the targeting ligand binds to a cell surface antigen expressed on the surface of a target cell. In some embodiments, the targeting ligand binds to a carbohydrate, a polypeptide or glycoprotein expressed on the surface of a target cell. In some embodiments, the targeting ligand is a lectin or an integrin. In some embodiments, the targeting ligand is an antibody. In some embodiments, the targeting ligand is a targeting non-antibody. In some embodiments, the targeting ligand is a co-stimulatory molecule.

In some embodiments, the targeting ligand is a ligand that binds to a cell surface molecule of a diseased cell. In some embodiments, the targeting ligand is a ligand that binds to a cell surface molecule that is specific to the diseased cell. In some embodiments, the targeting ligand is a ligand that binds to a cell surface molecule that is upregulated (i.e., has a higher expression) on the diseased cell compared to non-diseased cells. In some embodiments, the targeting ligand is an antibody. In some embodiments, the diseased cell is a cancer cell. In some embodiments, the targeting ligand is a ligand that binds to a cell surface molecule expressed by a hematopoietic cell. In some embodiments, the targeting ligand targets an inflammatory cell (e.g., neutrophil, macrophage, monocyte, eosinophil, basophil). In some embodiments, the targeting ligand targets a cell involved in an autoimmune disease (e.g. a lymphocyte, such as a B lymphocyte or a T lymphocyte).

In some embodiments, the targeting ligand is a ligand that binds to a cell surface molecule of a cancer cell. In some embodiments, the targeting ligand is a ligand that binds to a cell surface molecule that is specific to the cancer cell. In some embodiments, the targeting ligand is a ligand that binds to a cell surface molecule that is upregulated (i.e., has a higher expression) on the cancer cell compared to non-cancer cells. In some embodiments, targeting ligand is a ligand that binds to a cell surface molecule that that is expressed by the cancer cell but not a non-cancer cell. In some embodiments, the targeting ligand is a ligand that binds to a tumor antigen. In some embodiments, the targeting ligand is a ligand that binds to a cell surface receptor that is upregulated in the tumor cell (i.e., has a higher expression) compared to a non-tumor cell. In some embodiments, the targeting ligand is a ligand that binds to a cell surface receptor that that is expressed by the tumor cell but not a non-tumor cell. In some embodiments, the targeting ligand is an antibody.

In some embodiments, the targeting antibody used in the compositions and methods provided herein is an antibody that binds to a cell surface molecule on the targeted cell. In some embodiments, the targeting antibody is an antibody that binds to a cell surface molecule that is specific for the targeted cell. In some embodiments, the targeting antibody is an antibody that binds to a cell surface molecule that is upregulated (i.e., has a higher expression) on the target cell compared to non-targeted cells.

In some embodiments, the targeting antibody is an antibody that binds to a cell surface molecule of a diseased cell. In some embodiments, the targeting antibody is an antibody that binds to a cell surface molecule that is specific to the diseased cell. In some embodiments, the targeting antibody is an antibody that binds to a cell surface molecule that is upregulated (i.e., has a higher expression) on the diseased cell compared to non-diseased cells.

In some embodiments, the targeting antibody is an antibody that binds to a cell surface molecule of a cancer cell. In some embodiments, the targeting antibody is an antibody that binds to a cell surface molecule that is specific to the cancer cell. In some embodiments, the targeting antibody is an antibody that binds to a cell surface molecule that is upregulated (i.e., has a higher expression) on the cancer cell compared to non-cancer cells. In some embodiments, the targeting antibody binds to a cell surface molecule that that is expressed by the cancer cell but not a non-cancer cell. In some embodiments, the antibody binds to a tumor antigen. In some embodiments, the targeting antibody binds to a cell surface receptor that is upregulated in the tumor cell (i.e., has a higher expression) compared to a non-tumor cell. In some embodiments, the targeting antibody binds to a cell surface receptor that that is expressed by the tumor cell but not a non-tumor cell.

In some embodiments, the targeting antibody is a tumor specific antibody. In some embodiments, the targeting antibody binds to CD3, CD19, CD20, CD22, CD25, CD30, CD33, CD52, interleukin-2 receptor (IL-2). HLA-DR10β, tenascin, CEA, MUC1, TAG72, EBBB2 receptor (HER2), CD56 (NCAM), CS-125, Cripto, glycoprotein NMB (osteoactivin), CD70, prostate specific membrane antigen (PSMA), SLC44A4 (AGS-5), folate receptor, an integrin, such as αvβ3-integrin, transferrin receptor, granulocyte-macrophage colony-stimulating factor (GM-CSF) receptor, aminopeptidase N (CD13), galactosamine receptor, leutenizing hormone releasing hormone (LHRH) receptor, vascular endothelial growth factor (VEGF) receptor (FLK1), ROR1, mesothelin, CD33/IL3Ra, c-Met; PSMA, Glycolipid F77, EGFRvlII, GD-2, NY-ESO-1 TCR, MAGE A3 TCR.

In some embodiments, the targeting antibody is gemtuzumab, inotuumab, trastuzumab (Herceptin), HD37, M195, LMB2, lym1, 81C6, HMFG1, CC49, rituximab, epratuzumab, lorvotuzumab, 2C3, imgn388, SAR3419, Bi1B062, brentixumab, glembatumumab, SGN-75, PSMA ADC, ASG-5ME or mdx-1203. In some embodiments, the targeting antibody is a variant of gemtuzumab, inotuumab, trastuzumab (Herceptin), HD37, M195, LMB2, lym1, 81C6, HMFG1, CC49, rituximab, epratuzumab, lorvotuzumab, 2C3, imgn388, SAR3419, Bi1B062, brentixumab, glembatumumab, SGN-75, PSMA ADC, ASG-5ME or mdx-1203. In some embodiments, the comprises an antigen-binding fragment of gemtuzumab, inotuumab, trastuzumab (Herceptin), HD37, M195, LMB2, lym1, 81C6, HMFG1, CC49, rituximab, epratuzumab, lorvotuzumab, 2C3, imgn388, SAR3419. Bi1B062, brentixumab, glembatumumab, SGN-75, PSMA ADC, ASG-5ME or mdx-1203.

In some embodiments, the targeting ligand is a non-antibody ligand that binds to a receptor. In some embodiments, the targeting ligand binds to folate receptor, αvβ3-integrin, transferrin receptor, GM-CSF receptor, aminopeptidase N (CD13), galactosamine receptor and LHRH receptor. In some embodiments, the targeting ligand comprises RGD, NGR, folate, transferrin, GM-CSF, or galactosamine.

In some embodiments, the targeting antibody is natural antibody. In some embodiments, the targeting antibody is a synthetic antibody. In some embodiments, the targeting antibody is a recombinant antibody. In some embodiments, the targeting antibody is an antibody fragment containing at least a portion of the variable region of the immunoglobulin molecule that retains the binding specificity ability of the full-length immunoglobulin. In some embodiments, the targeting antibody is any protein having a binding domain that is homologous or substantially homologous to an immunoglobulin antigen-binding domain (antibody combining site). In some embodiments, the targeting antibody is a multispecific antibodies (e.g., bispecific antibodies). In some embodiments, the targeting antibody is a human antibody or non-human antibody. In some embodiments, the targeting antibody is a humanized antibody. In some embodiments, the targeting antibody is a chimeric antibody. In some embodiments, the targeting antibody is an intrabody. In some embodiments, the targeting antibody is an antibody fragment, such as, but not limited to, Fab fragment, Fab′ fragment, F(ab′)₂ fragment, Fv fragment, disulfide-linked Fv (dsFv), Fd fragment, Fd′ fragment, single-chain Fv (scFv), single-chain Fab (scFab), diabody, anti-idiotypic (anti-Id) antibody, or antigen-binding fragments of any of the above In some embodiments, the targeting antibody is any member of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

In some embodiments, the targeting antibody is a monoclonal antibody. In some embodiments, the targeting antibody is an IgG antibody. In some embodiments, the targeting antibody is a monovalent antibody. In some embodiments, the targeting antibody is multivalent antibody. In some embodiments, the targeting antibody is a bivalent antibody. In some embodiments, the targeting antibody is an antibody fragment, such as a single-chain variable fragment (scFv). In some embodiments, the targeting antibody is a humanized antibody. In some embodiments, the targeting antibody is a variant of a known tumor specific antibody. In some embodiments, the targeting antibody is an antigen-binding fragment of a known tumor specific antibody.

In some embodiments, the SDM and/or therapeutic cargo is conjugated to a portion of the antibody such that conjugation does not interfere with antibody-antigen binding. In some embodiments, the SDM and/or therapeutic cargo is conjugated to the Fc portion of the antibody.

### Selective Delivery Molecules

Disclosed herein, in certain embodiments, are selective delivery molecules (SDMs) for use in the compositions and methods described herein. In certain embodiments, the SDMs described herein are used alone or are conjugated to one or more molecules. In certain embodiments, the SDMs described herein are conjugated to a targeting ligand. In some embodiments, the targeting ligand is an antibody. In certain embodiments, the SDMs described herein are conjugated to an existing antibody-drug conjugate. In certain embodiments, the SDMs described herein comprise a therapeutic cargo. In certain embodiments, the SDMs described herein comprising a therapeutic cargo are conjugated to a targeting ligand. In some embodiments, the targeting ligand is an antibody.

In certain embodiments, the SDM is an SDM of Formula I, having the structure:

A-X-B-[c_{B}-D_{B}] Formula I

wherein,

X is a cleavable linker:

A is a peptide with a sequence comprising 5 to 9 acidic amino acids;

B is a peptide with a sequence comprising 7 to 9 basic amino acids;

c_{B} comprises 0-1 amino acid;

D_{B} is a therapeutic agent; and

wherein [c_{B}-D_{B}] is bound to any amino acid of B. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} is selected from a naturally-occurring amino acid or a non-naturally-occurring amino acid. In some embodiments, c_{B} is selected from a D amino acid, a L amino acid, an α-amino acid, a β-amino acid, or a γ-amino acid. In some embodiments, c_{B} is selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} is selected from D-cysteine, D-glutamate, lysine, and para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments. X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7). ESPAYYTA (SEQ ID NO: 8), DPRSFL (SEQ ID NO: 9). PPRSFL (SEQ ID NO: 10), RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments. X comprises the amino acid sequence RPLALWRS (SEQ ID NO: 7). In some embodiments, X comprises the amino acid sequence DPRSFL (SEQ ID NO: 9). In some embodiments, X comprises the amino acid sequence RLQLKL (SEQ ID NO: 11). In some embodiments, X comprises the amino acid sequence RLQLK(Ac) (SEQ ID NO: 12). In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula I. In some embodiments, the selective delivery molecule of Formula I is: SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-18, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

In certain embodiments, the SDM is an SDM of Formula II, having the structure:

A-[c_{M}-M]-X-B-[c_{B}-D_{B}] Formula II

wherein,

X is a cleavable linker:

A is a peptide with a sequence comprising 5 to 9 acidic amino acids;

B is a peptide with a sequence comprising 7 to 9 basic amino acids;

c_{B} and c_{M} each independently comprise 0-1 amino acid;

M is a macromolecule;

D_{B} is a therapeutic agent; and

wherein [c_{M}-M] is bound to at any position of A or X, and [c_{B}-D_{B}] is bound to any amino acid of B. In some embodiments, the therapeutic agent is cortisone. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B} and c_{M} are each independently selected from a naturally-occurring amino acid or a non-naturally-occurring amino acid. In some embodiments, c_{B} and c_{M} are each independently selected from a D amino acid, a L amino acid, an α-amino acid, a β-amino acid, or γ-amino acid. In some embodiments, c_{B} and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} and c_{M} are each independently selected from D-cysteine, D-glutamate, lysine, and para-4-acetyl L-phenylalanine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by an extracellular protease. In some embodiments, X is cleavable by a soluble protease or cell surface associated protease. In some embodiments, X is cleavable by a matrix metalloproteinase. In some embodiments. X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, X comprises the amino acid sequence DPRSFL (SEQ ID NO: 9). In some embodiments, X comprises the amino acid sequence RLQLKL (SEQ ID NO: 11). In some embodiments. X comprises the amino acid sequence RLQLK(Ac) (SEQ ID NO: 12). In some embodiments, M is selected from a protein, a natural polymer, a synthetic polymer, or a dendrimer. In some embodiments, M is selected from dextran, a PEG polymer, albumin, or a combination thereof. In some embodiments. M is a PEG. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula II. In some embodiments, the selective delivery molecule of Formula II is: SDM-101, SDM-102, SDM-103, SDM-104, SDM-105, SDM-106, SDM-107, SDM-108, SDM-109, SDM-10, SDM-111, SDM-112, SDM-13, SDM-14, SDM-15, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

In certain embodiments, the SDM does not comprise a cargo molecule. In some embodiments, an SDM without a cargo molecule is conjugated to an existing antibody-drug conjugate (e.g. Form 3, FIG. 3). In certain embodiments, the SDM is an SDM of Formula III or IV, having the structure:

A-X-B Formula III

A-[c_{M}-M]-X-B Formula IV

wherein,

X is a cleavable linker;

A is a peptide with a sequence comprising 5 to 9 acidic amino acids;

B is a peptide with a sequence comprising 7 to 9 basic amino acids;

c_{M} comprises 0-1 amino acid;

M is a macromolecule; and

wherein [c_{M}-M] is bound to at any position of A or X. In some embodiments, A and B do not have an equal number of acidic and basic amino acids. In some embodiments, the number of basic amino acids in B is greater than the number of acidic amino acids in A. In some embodiments. A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{M} is selected from a naturally-occurring amino acid or a non-naturally-occurring amino acid. In some embodiments, c_{M} is selected from a D amino acid, a L amino acid, an α-amino acid, a β-amino acid, or γ-amino acid. In some embodiments, c_{M} is selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{M}N is selected from D-cysteine, D-glutamate, lysine, and para-4-acetyl L-phenylalanine. In some embodiments, c_{M} is any amino acid with a side chain capable of forming an oxime or hydrazone bond upon reaction with a hydroxylamine or hydrazine group. In some embodiments, c_{M1} is para-4-acetyl L-phenylalanine. In some embodiments, X is cleavable by a protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10). RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, X comprises the amino acid sequence DPRSFL (SEQ ID NO: 9). In some embodiments, X comprises the amino acid sequence RLQLKL (SEQ ID NO: 11). In some embodiments, X comprises the amino acid sequence RLQLK(Ac) (SEQ ID NO: 12). In some embodiments. M is selected from a protein, a natural polymer, a synthetic polymer, or a dendrimer. In some embodiments, M is selected from dextran, a PEG polymer, albumin, or a combination thereof. In some embodiments, M is a PEG. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula III or IV. In some embodiments, the selective delivery molecule of Formula III or IV is: SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

Dual Protease Substrate SDMs

Described herein, in certain embodiments, is an SDM that comprises more than one protease cleavage site. In some embodiments, the SDM comprises a cleavage site for an extracellular protease and a cleavage site for an intracellular protease. In some embodiments, the SDM comprises a cleavable linker X and a cleavable linker Y, where linker X comprises a cleavage site for an extracellular protease and linker Y comprises a cleavage site for an intracellular protease. In some embodiments, linker X is located between portion of A and portion of B, and linker Y is located between portion of B and the therapeutic cargo. In some embodiments, the intracellular protease that cleaves the Y linker is a lysosomal protease. In some embodiments, the protease is one that is activated at low pH. In some embodiments, the protease is one that is activated at low pH and is expressed in an endosome. In some embodiments, the protease is a cathepsin. In some embodiments, the cathepsin is cathepsin B. In some embodiments, the Y linker additionally comprises a self-immolative cleavage site located between the intracellular protease cleavage site and the attached therapeutic cargo. In some embodiments, the self-immolative cleavage site is a PABC (p-aminobenzylcarbonyl) spacer and its analogs thereof. In some embodiments, the self-immolative cleavage site is a thiazole containing linker.

In certain embodiments, the SDM is an SDM of Formula V, having the structure:

A-[c_{M}-M]-X-B-Y-[c_{B}-D_{B}]Formula V

wherein,

X is a cleavable linker:

Y is a cleavable linker:

A is a peptide with a sequence comprising 5 to 9 acidic amino acids:

B is a peptide with a sequence comprising 7 to 9 basic amino acids;

c_{B} and c_{M} each independently comprise 0-1 amino acid;

M is a macromolecule;

D_{B} is a therapeutic agent; and

wherein [c_{M}-M] is bound to at any position of A or X, and [c_{B}-D_{B}] is bound to any amino acid of B. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{B}, and c_{M} are each independently a 0-1 amino acid. In some embodiments, c_{B} and c_{M} are each independently selected from a naturally-occurring amino acid or a non-naturally-occurring amino acid. In some embodiments, c_{B} and c_{M} are each independently selected from a D amino acid, a L amino acid, an α-amino acid, a β-amino acid, or a γ-amino acid. In some embodiments, c_{B} and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{B} and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2). PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, X comprises the amino acid sequence DPRSFL (SEQ ID NO: 9). In some embodiments, X comprises the amino acid sequence RLQLKL (SEQ ID NO: 11). In some embodiments, X comprises the amino acid sequence RLQLK(Ac) (SEQ ID NO: 12). In some embodiments, M is selected from a protein, a natural polymer, a synthetic polymer, or a dendrimer. In some embodiments, M is selected from dextran, a PEG polymer, albumin, or a combination thereof. In some embodiments, M is a PEG. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), and approximately 40 kDa (PEG 40 kDa)). In some embodiments, Y is cleavable by a protease. In some embodiments. Y is cleavable by an intracellular protease. In some embodiments, Y comprises an amino acid sequence that is cleavable by Cathepsin B. In some embodiments, Y comprises the amino acid sequence Phe-Lys or Val-Cit (L-citrulline). In some embodiments, Y comprises a site for self-immolative cleavage. In some embodiments, Y comprises a PABC self-immolative spacer. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula V. In some embodiments, the selective delivery molecule of Formula V is: SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153.

Other SDMs

In certain embodiments, the SDM is an SDM comprising an imaging agent. In certain embodiments, the SDM is an SDM comprising an imaging agent and a therapeutic agent. In certain embodiments, the SDM is an SDM comprising two or more imaging agents or two or more therapeutic agents.

In certain embodiments, the SDM is an SDM comprising two or more imaging agents for Forster resonance energy transfer (FRET) imaging, where one imaging agent is conjugated to the A portion of the SDM and one imaging agent is conjugated to the B portion of the SDM.

In certain embodiments, the SDM is an SDM of Formula VI, having the structure:

[D_{A}-c_{A}]-A-[c_{M}-M]-X-B-[c_{B}-D_{B}] Formula VI

wherein,

X is a cleavable linker;

A is a peptide with a sequence comprising 5 to 9 acidic amino acids:

B is a peptide with a sequence comprising 7 to 9 basic amino acids;

c_{A}, c_{B}, and c_{M} each independently comprise 0-1 amino acid;

M is a macromolecule; and

D_{A} and D_{B} are each independently selected from an imaging agent and a therapeutic; and

wherein [c_{M}-M] is bound to at any position of A or X, [D_{A}-c_{A}] is bound to any amino acid of A, and [c_{B}-D_{B}] is bound to any amino acid of B. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{A}, c_{B}, and c_{M} are each independently a 0-1 amino acid. In some embodiments, c_{A}, c_{B}, and c_{M} are each independently selected from a naturally-occurring amino acid or a non-naturally-occurring amino acid. In some embodiments, c_{A}, c_{B}, and c_{M} are each independently selected from a D amino acid, a L amino acid, an α-amino acid, a β-amino acid, or γ-amino acid. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{A} is lysine. In some embodiments, c_{M} is any amino acid with a side chain capable of forming an oxime or hydrazone bond upon reaction with a hydroxylamine or hydrazine group. In some embodiments, c_{A1} is para-4-acetyl L-phenylalanine. In some embodiments, X is cleavable by a protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, X comprises the amino acid sequence DPRSFL (SEQ ID NO: 9). In some embodiments. X comprises the amino acid sequence RLQLKL (SEQ ID NO: 11). In some embodiments, X comprises the amino acid sequence RLQLK(Ac) (SEQ ID NO: 12). In some embodiments, M is selected from a protein, a natural polymer, a synthetic polymer, or a dendrimer. In some embodiments, M is selected from dextran, a PEG polymer, albumin, or a combination thereof. In some embodiments, M is a PEG. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, D_{A} and D_{B} are a pair of acceptor and donor fluorescent moieties that are capable of undergoing Försters/fluorescence resonance energy transfer with the other. In some embodiments, D_{A} and D_{B} are Cy5 and Cy7. In some embodiments, D_{A} and D_{B} are Cy5 and IRDye750. In some embodiments, D_{A} and D_{B} are Cy5 and IRDye800. In some embodiments, D_{A} and D_{B} are Cy5 and ICG. In some embodiments, D_{A} and D_{B} are a fluorescent moiety and a fluorescence-quenching moiety. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula VI. In some embodiments, the molecule of Formula I is: SDM-14, SDM-15, SDM-23, SDM-24, SDM-25, SDM-26, SDM-27, SDM-32, or SDM-35.

In certain embodiments, the SDM is an SDM of Formula VI, having the structure:

[D_{A}-c_{A}]-A-[c_{M}-M]-X-B-[c_{B}-D_{B}]Formula VI

wherein,

X is a cleavable linker;

A is a peptide with a sequence comprising 5 to 9 acidic amino acids:

B is a peptide with a sequence comprising 7 to 9 basic amino acids;

c_{A}, c_{B}, and c_{M} each independently comprise 0-1 amino acid:

M is a polyethylene glycol (PEG) polymer; and

D_{A} and D_{B} are each independently an imaging agent; and

wherein [c_{M}-M] is bound to at any position of A or X, [D_{A}-c_{A}] is bound to any amino acid of A, and [c_{B}-D_{B}] is bound to any amino acid of B. In some embodiments, A and B do not have an equal number of acidic and basic amino acids. In some embodiments, the number of basic amino acids in B is greater than the number of acidic amino acids in A. In some embodiments, A is a peptide comprising 5 or 9 consecutive glutamates (SEQ ID NOS 3 and 4, respectively). In some embodiments, B is a peptide comprising 8 or 9 consecutive arginines (SEQ ID NOS 5 and 6, respectively). In some embodiments, A is a peptide comprising 5 or 9 consecutive glutamates (SEQ ID NOS 3 and 4, respectively) and B is a peptide comprising 8 or 9 consecutive arginines (SEQ ID NOS 5 and 6, respectively). In some embodiments, A is a peptide comprising 5 consecutive glutamates (SEQ ID NO: 3) and B is a peptide comprising 8 consecutive arginines (SEQ ID NO: 5). In some embodiments, c_{A}, c_{B}, and c_{M} are each independently a 0-1 amino acid. In some embodiments, c_{A}, c_{B}, and c_{M} are each independently selected from a naturally-occurring amino acid or a non-naturally-occurring amino acid. In some embodiments, c_{A}, c_{B}, and c_{M} are each independently selected from a D amino acid, a L amino acid, an α-amino acid, a 6-amino acid, or γ-amino acid. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{A} is lysine. In some embodiments, c a is any amino acid with a side chain capable of forming an oxime or hydrazone bond upon reaction with a hydroxylamine or hydrazine group. In some embodiments, c_{M} is para-4-acetyl L-phenylalanine. In some embodiments, X is cleavable by a protease. In some embodiments, X is cleavable by a matrix metalloproteinase. In some embodiments, X comprises an amino acid sequence that is cleavable by MMP2, MMP7, MMP9, or MMP14. In some embodiments, X comprises a peptide linkage. In some embodiments, X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12). In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, X comprises the amino acid sequence DPRSFL (SEQ ID NO: 9). In some embodiments. X comprises the amino acid sequence PPRSFL (SEQ ID NO: 10). In some embodiments, X comprises the amino acid sequence RLQLKL (SEQ ID NO: 11). In some embodiments, X comprises the amino acid sequence RLQLK(Ac) (SEQ ID NO: 12). In some embodiments, D_{A} and D_{B} are a pair of acceptor and donor fluorescent moieties that are capable of undergoing Försters/fluorescence resonance energy transfer with the other. In some embodiments, D_{A} and D_{B} are Cy5 and Cy7. In some embodiments, D_{A} and D_{B} are Cy5 and IRDye750. In some embodiments, D_{A} and D_{B} are Cy5 and IRDye800. In some embodiments, D_{A} and D_{B} are Cy5 and ICG. In some embodiments, D_{A} and D_{B} are a fluorescent moiety and a fluorescence-quenching moiety. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula VI. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), and approximately 40 kDa (PEG 40 kDa)). In some embodiments, the molecule of Formula VI is: SDM-14, SDM-15, SDM-23, SDM-24, SDM-25, SDM-26, SDM-27, SDM-32; or SDM-35.

In certain embodiments, the SDM is an SDM of Formula VI, having the structure:

[D_{A}-c_{A}]-A-[c_{M}-M]-X-B-[c_{B}-D_{B}] Formula VI

wherein,

X is a peptide linker cleavable by a matrix metalloproteinase;

A is a peptide with a sequence comprising 5 or 9 consecutive glutamates (SEQ ID NOS 3 and 4, respectively);

B is a peptide with a sequence comprising 8 or 9 consecutive arginines (SEQ ID NOS 5 and 6, respectively);

c_{A}, c_{B}, and c_{M} each independently comprise 0-1 amino acid:

M is a polyethylene glycol (PEG) polymer; and

D_{A} and D_{B} are a pair of acceptor and donor fluorescent moieties that are capable of undergoing Försters/fluorescence resonance energy transfer with the other; and wherein [c_{M}-M] is bound to at any position of A or X, [D_{A}-c_{A}] is bound to any amino acid of A, and [c_{B}-D_{B}] is bound to any amino acid of B. In some embodiments, A and B do not have an equal number of acidic and basic amino acids. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently a 0-1 amino acid. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X comprises the amino acid sequence PLGLAG (SEQ ID NO: 2). In some embodiments, X comprises the amino acid sequence PLG-C(me)-AG (SEQ ID NO: 1). In some embodiments. D_{A} and D_{B} are Cy5 and Cy7. In some embodiments, D_{A} and D_{B} are Cy5 and Cy7. In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 100 kDa (PEG 100 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 400 kDa (PEG 40 kDa)). In certain embodiments, the SDM is an SDM comprising the structure of Formula VI.

In certain embodiments, the SDM is an SDM of Formula VI, having the structure:

[D_{A}-c_{A}]-A-[c_{M}-M]-X-B-[c_{B}-D_{B}] Formula VI

wherein,

X is a peptide linker cleavable by a matrix metalloproteinase:

A is a peptide with a sequence comprising 5 consecutive glutamates (SEQ ID NO: 3);

B is a peptide with a sequence comprising 8 consecutive arginines (SEQ ID NO: 5):

c_{A}, c_{B}, and c_{M} each independently comprise 0-1 amino acid:

M is a polyethylene glycol (PEG) polymer; and

D_{A} and D_{B} are a pair of acceptor and donor fluorescent moieties that are capable of undergoing Försters/fluorescence resonance energy transfer with the other; and wherein [c_{M}-M] is bound to at any position of A or X, [D_{A}-c_{A}] is bound to any amino acid of A, and [c_{B}-D_{B}] is bound to any amino acid of B. In some embodiments, c_{A}, c_{B}, and c_{M} are each independently a 0-1 amino acid. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine. In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, D_{A} and D_{B} are Cy5 and Cy7. In some embodiments, D_{A} and D_{B} are Cy5 and IRDye750. In some embodiments, D_{A} and D_{B} are Cy5 and IRDye800. In some embodiments, D_{A} and D_{B} are Cy5 and ICG. In some embodiments, M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 400 kDa (PEG 40 kDa)). In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula VI.

In certain embodiments, the SDM is an SDM of Formula VI, having the structure:

[D_{A}-c_{A}]-A-[c_{M}-M]-X-B-[c_{B}-D_{B}] Formula VI

wherein,
X is a peptide linker cleavable by a matrix metalloproteinase;
A is a peptide with a sequence comprising 9 consecutive glutamates (SEQ ID NO: 4);
B is a peptide with a sequence comprising 9 consecutive arginines (SEQ ID NO: 6);
c_{A}, c_{B}, and c_{M} each independently comprise 0-1 amino acid;
M is a polyethylene glycol (PEG) polymer; and
D_{A} and D_{B} are a pair of acceptor and donor fluorescent moieties that are capable of undergoing Försters/fluorescence resonance energy transfer with the other; and
   wherein [c_{M}-M] is bound to at any position of A or X, [D_{A}-c_{A}] is bound to any amino acid of A, and [c_{B}-D_{B}] is bound to any amino acid of B. In some embodiments, c_{A}, c_{B}, and c_{M} are each independently a 0-1 amino acid. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine.

In some embodiments, X comprises the amino acid sequence PLGLAG (SEQ ID NO: 2). In some embodiments, X comprises the amino acid sequence PLG-C(me)-AG (SEQ ID NO: 1). In some embodiments, X comprises the amino acid sequence RPLALWRS (SEQ ID NO: 7). In some embodiments, D_{A} and D_{B} are Cy5 and Cy7. In some embodiments, D_{A} and DE are Cy5 and IRDye750. In some embodiments, D_{A} and D_{B} are Cy5 and IRDye800. In some embodiments, D_{A} and D_{B} are Cy5 and ICG. In some embodiments. M is selected from a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)). In some embodiments, A comprises a thiol reactive group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo. In certain embodiments, the SDM is an SDM comprising the structure of Formula VI.

In some embodiments, the SDM comprises a structure selected from: SDM-1, SDM-2, SDM-3, SDM-4, SDM-5, SDM-6, SDM-7, SDM-8, SDM-9, SDM-10, SDM-11, SDM-12, SDM-13, SDM-14, SDM-15, SDM-16, SDM-17, SDM-18, SDM-19, SDM-20, SDM-21, SDM-22, SDM-23, SDM-24, SDM-25, SDM-26, SDM-27, SDM-28, SDM-29, SDM-30, SDM-31, SDM-32, SDM-33, SDM-34, SDM-35, SDM-36, SDM-37, SDM-38, SDM-39, and SDM-40 (see International PCT Pub. No. WO2013/019681). In some embodiments, the selective delivery molecule comprises a structure selected from: SDM-14, SDM-15, SDM-23, SDM-24, SDM-25, SDM-26, SDM-27, SDM-32, or SDM-35. In certain embodiments, the selective delivery molecule comprises Peptide P-3 (see International PCT Pub. No. WO2013/019681).

### Portion of A

In some embodiments, A is a peptide with a sequence comprising 2 to 20 acidic amino acids. In some embodiments, peptide portion of A comprises between about 2 to about 20 acidic amino acids. In some embodiments, peptide portion of A comprises between about 5 to about 20 acidic amino acids. In some embodiments, A has a sequence comprising 5 to 9 acidic amino acids. In some embodiments, A has a sequence comprising 5 to 8 acidic amino acids. In some embodiments, A has a sequence comprising 5 to 7 acidic amino acids. In some embodiments, A has a sequence comprising 5 acidic amino acids. In some embodiments, A has a sequence comprising 6 acidic amino acids. In some embodiments, A has a sequence comprising 7 acidic amino acids. In some embodiments, A has a sequence comprising 8 acidic amino acids. In some embodiments. A has a sequence comprising 9 acidic amino acids.

In some embodiments, peptide portion of A comprises between about 2 to about 20 consecutive acidic amino acids. In some embodiments, peptide portion of A comprises between about 5 to about 20 consecutive acidic amino acids. In some embodiments, A has a sequence comprising 5 to 9 consecutive acidic amino acids. In some embodiments, A has a sequence comprising 5 to 8 consecutive acidic amino acids. In some embodiments, A has a sequence comprising 5 to 7 consecutive acidic amino acids. In some embodiments, A has a sequence comprising 5 consecutive acidic amino acids. In some embodiments, A has a sequence comprising 6 consecutive acidic amino acids. In some embodiments, A has a sequence comprising 7 consecutive acidic amino acids. In some embodiments, A has a sequence comprising 8 consecutive acidic amino acids. In some embodiments, A has a sequence comprising 9 consecutive acidic amino acids.

In some embodiments, peptide portion of A comprises between about 2 to about 20 acidic amino acids selected from, aspartates and glutamates. In some embodiments, peptide portion of A comprises between about 5 to about 20 acidic amino acids selected from, aspartates and glutamates. In some embodiments, A has a sequence comprising 5 to 9 acidic amino acids selected from, aspartates and glutamates. In some embodiments, A has a sequence comprising 5 to 8 acidic amino acids selected from, aspartates and glutamates. In some embodiments. A has a sequence comprising 5 to 7 acidic amino acids selected from, aspartates and glutamates. In some embodiments, A has a sequence comprising 5 acidic amino acids selected from, aspartates and glutamates. In some embodiments, A has a sequence comprising 6 acidic amino acids selected from, aspartates and glutamates. In some embodiments, A has a sequence comprising 7 acidic amino acids selected from, aspartates and glutamates. In some embodiments, A has a sequence comprising 8 acidic amino acids selected from, aspartates and glutamates. In some embodiments, A has a sequence comprising 9 acidic amino acids selected from, aspartates and glutamates.

In some embodiments, peptide portion of A comprises between about 2 to about 20 consecutive acidic amino acids selected from, aspartates and glutamates. In some embodiments, peptide portion of A comprises between about 5 to about 20 consecutive acidic amino acids selected from, aspartates and glutamates. In some embodiments. A has a sequence comprising 5 to 9 consecutive acidic amino acids selected from, aspartates and glutamates. In some embodiments, A has a sequence comprising 5 to 8 consecutive acidic amino acids selected from, aspartates and glutamates. In some embodiments, A has a sequence comprising 5 to 7 consecutive acidic amino acids selected from, aspartates and glutamates. In some embodiments, A has a sequence comprising 5 consecutive acidic amino acids selected from, aspartates and glutamates. In some embodiments, A has a sequence comprising 6 consecutive acidic amino acids selected from, aspartates and glutamates. In some embodiments, A has a sequence comprising 7 consecutive acidic amino acids selected from, aspartates and glutamates. In some embodiments, A has a sequence comprising 8 consecutive acidic amino acids selected from, aspartates and glutamates. In some embodiments, A has a sequence comprising 9 consecutive acidic amino acids selected from, aspartates and glutamates.

In some embodiments, peptide portion of A comprises between about 2 to about 20 glutamates. In some embodiments, peptide portion of A comprises between about 5 to about 20 glutamates. In some embodiments, A has a sequence comprising 5 to 9 glutamates (SEQ ID NO: 13). In some embodiments, A has a sequence comprising 5 to 8 glutamates (SEQ ID NO: 14). In some embodiments, A has a sequence comprising 5 to 7 glutamates (SEQ ID NO: 15). In some embodiments, A has a sequence comprising 5 glutamates (SEQ ID NO: 3). In some embodiments, A has a sequence comprising 6 glutamates (SEQ ID NO: 16). In some embodiments, A has a sequence comprising 7 glutamates (SEQ ID NO: 17). In some embodiments, A has a sequence comprising 8 glutamates (SEQ ID NO: 18). In some embodiments. A has a sequence comprising 9 glutamates (SEQ ID NO: 4).

In some embodiments, peptide portion of A comprises between about 2 to about 20 consecutive glutamates. In some embodiments, peptide portion of A comprises between about 5 to about 20 consecutive glutamates. In some embodiments, A has a sequence comprising 5 to 9 consecutive glutamates (SEQ ID NO: 13). In some embodiments, A has a sequence comprising 5 to 8 consecutive glutamates (SEQ ID NO: 14). In some embodiments, A has a sequence comprising 5 to 7 consecutive glutamates (SEQ ID NO: 15). In some embodiments, A has a sequence comprising 5 consecutive glutamates (SEQ ID NO: 3). In some embodiments, A has a sequence comprising 6 consecutive glutamates (SEQ ID NO: 16). In some embodiments, A has a sequence comprising 7 consecutive glutamates (SEQ ID NO: 17). In some embodiments, A has a sequence comprising 8 consecutive glutamates (SEQ ID NO: 18). In some embodiments, A has a sequence comprising 9 consecutive glutamates (SEQ ID NO: 4).

In some embodiments, portion of A comprises 5 consecutive glutamates (i.e., EEEEE or eeeee) (SEQ ID NO: 3). In some embodiments, portion of A comprises 9 consecutive glutamates (i.e., EEEEEEEEE or eeeeeeeee) (SEQ ID NO: 4).

An acidic portion of A may include amino acids that are not acidic. Acidic portion of A may comprise other moieties, such as negatively charged moieties. In embodiments of a selective delivery molecule disclosed herein, an acidic portion of A may be a negatively charged portion, preferably having about 2 to about 20 negative charges at physiological pH that does not include an amino acid.

In some embodiments, the amount of negative charge in portion of A is approximately the same as the amount of positive charge in portion of B. In some embodiments, the amount of negative charge in portion of A is not the same as the amount of positive charge in portion of B.

In some embodiments, improved tissue uptake is seen in a selective delivery molecule wherein the amount of negative charge in portion of A is not the same as the amount of positive charge in portion of B. In some embodiments, improved solubility is observed in a selective delivery molecule wherein the amount of negative charge in portion of A is not the same as the amount of positive charge in portion of B. In some embodiments, faster tissue uptake is seen in a selective delivery molecule wherein the amount of negative charge in portion of A is not the same as the amount of positive charge in portion of B. In some embodiments, greater tissue uptake is seen in a selective delivery molecule wherein the amount of negative charge in portion of A is not the same as the amount of positive charge in portion of B.

Portion of A is either L-amino acids or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, portion of A comprises a thiol reactive group. In some embodiments, the thiol group is an N-terminal thiol group. In some embodiments, the thiol reactive group is selected from among haloacetyls, maleimides, aziridines, acryloyls, arylating agents, vinylsulfones, pyridyl disulfides, TNB-thiols and disulfide reducing agents. In some embodiments, the thiol reactive group covalently binds to a carrier protein. In some embodiments, the carrier protein is albumin. In some embodiments, the thiol reactive group covalently binds to Cysteine 34 of albumin. In some embodiments, the thiol reactive group covalently binds to albumin in vivo.

It will be understood that portion of A may include non-standard amino acids, such as, for example, hydroxylysine, desmosine, isodesmosine, or other non-standard amino acids. Portion of A may include modified amino acids, including post-translationally modified amino acids such as, for example, methylated amino acids (e.g., methyl histidine, methylated forms of lysine, etc.), acetylated Amino acids, amidated amino acids, formylated amino acids, hydroxylated amino acids, phosphorylated amino acids, or other modified amino acids. Portion of A may also include peptide mimetic moieties, including portions linked by non-peptide bonds and amino acids linked by or to non-amino acid portions.

The Selective Delivery Molecules disclosed herein are effective where A is at the amino terminus or where A is at the carboxy terminus, i.e., either orientation of the peptide bonds is permissible.

### Portion of B

In some embodiments, B is a peptide with a sequence comprising 5 to 15 basic amino acids. In some embodiments, peptide portion of B comprises between about 5 to about 20 basic amino acids. In some embodiments, peptide portion of B comprises between about 5 to about 12 basic amino acids. In some embodiments, peptide portion of B comprises between about 7 to about 9 basic amino acids. In some embodiments, peptide portion of B comprises between about 7 to about 8 basic amino acids. In some embodiments, peptide portion of B comprises 9 basic amino acids. In some embodiments, peptide portion of B comprises 8 basic amino acids. In some embodiments, peptide portion of B comprises 7 basic amino acids.

In some embodiments, peptide portion of B comprises between about 5 to about 20 consecutive basic amino acids. In some embodiments, peptide portion of B comprises between about 5 to about 12 consecutive basic amino acids. In some embodiments, peptide portion of B comprises between about 7 to about 9 consecutive basic amino acids. In some embodiments, peptide portion of B comprises between about 7 to about 8 consecutive basic amino acids. In some embodiments, peptide portion of B comprises 9 consecutive basic amino acids. In some embodiments, peptide portion of B comprises 8 consecutive basic amino acids. In some embodiments, peptide portion of B comprises 7 consecutive basic amino acids.

In some embodiments, peptide portion of B comprises between about 5 to about 20 basic amino acids selected from arginines, histidines, and lysines. In some embodiments, peptide portion of B comprises between about 5 to about 12 basic amino acids selected from arginines, histidines, and lysines. In some embodiments, peptide portion of B comprises between about 7 to about 9 basic amino acids selected from arginines, histidines, and lysines. In some embodiments, peptide portion of B comprises between about 7 to about 8 basic amino acids selected from arginines, histidines, and lysines. In some embodiments, peptide portion of B comprises 9 basic amino acids selected from arginines, histidines, and lysines. In some embodiments, peptide portion of B comprises 8 basic amino acids selected from arginines, histidines, and lysines. In some embodiments, peptide portion of B comprises 7 basic amino acids selected from arginines, histidines, and lysines.

In some embodiments, peptide portion of B comprises between about 5 to about 20 consecutive basic amino acids selected from arginines, histidines, and lysines. In some embodiments, peptide portion of B comprises between about 5 to about 12 consecutive basic amino acids selected from arginines, histidines, and lysines. In some embodiments, peptide portion of B comprises between about 7 to about 9 consecutive basic amino acids selected from arginines, histidines, and lysines. In some embodiments, peptide portion of B comprises between about 7 to about 8 consecutive basic amino acids selected from arginines, histidines, and lysines.

In some embodiments, peptide portion of B comprises 9 consecutive basic amino acids selected from arginines, histidines, and lysines. In some embodiments, peptide portion of B comprises 8 consecutive basic amino acids selected from arginines, histidines, and lysines. In some

In some embodiments, peptide portion of B comprises between about 5 to about 20 arginines. In some embodiments, peptide portion of B comprises between about 5 to about 12 arginines. In some embodiments, peptide portion of B comprises between about 7 to about 9 arginines. In some embodiments, peptide portion of B comprises between about 7 to about 8 arginines. In some embodiments, peptide portion of B comprises 9 arginines (SEQ ID NO: 6). In some embodiments, peptide portion of B comprises 8 arginines (SEQ ID NO: 5). In some embodiments, peptide portion of B comprises 7 arginines (SEQ ID NO: 19).

In some embodiments, peptide portion of B comprises between about 5 to about 20 consecutive arginines. In some embodiments, peptide portion of B comprises between about 5 to about 12 consecutive arginines. In some embodiments, peptide portion of B comprises between about 7 to about 9 consecutive arginines. In some embodiments, peptide portion of B comprises between about 7 to about 8 consecutive arginines. In some embodiments, peptide portion of B comprises 9 consecutive arginines (SEQ ID NO: 6). In some embodiments, peptide portion of B comprises 8 consecutive arginines (SEQ ID NO: 5). In some embodiments, peptide portion of B comprises 7 consecutive arginines (SEQ ID NO: 19).

A basic portion of B may include amino acids that are not basic. Basic portion of B may comprise other moieties, such as positively charged moieties. In embodiments, a basic portion of B may be a positively charged portion, preferably having between about 5 and about 20 positive charges at physiological pH, that does not include an amino acid. In some embodiments, the amount of negative charge in portion of A is approximately the same as the amount of positive charge in portion of B. In some embodiments, the amount of negative charge in portion of A is not the same as the amount of positive charge in portion of B.

Portion of B is either L-amino acids or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines.

It will be understood that portion of B may include non-standard amino acids, such as, for example, hydroxylysine, desmosine, isodesmosine, or other non-standard amino acids.

Portion of B may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion of B may also include peptide mimetic moieties, including portions linked by non-peptide bonds and amino acids linked by or to non-amino acid portions.

In embodiments where X is a peptide cleavable by a protease, it may be preferable to join the C-terminus of X to the N-terminus of B, so that the new amino terminus created by cleavage of X contributes an additional positive charge that adds to the positive charges already present in B.

### Conjugation Group (c)

In some embodiments, the cargo (e.g., D_{A} and D_{B}) and the macromolecule carriers (M) are attached indirectly to A-X-B.

In some embodiments, the cargo (e.g., D_{A} and D_{B}) and the macromolecule carriers (M) are attached indirectly to A-X-B by a conjugation group (c_{A}, c_{B}, and c_{M}). In some embodiments, the cargo (e.g., D_{A} and D_{B}) and the macromolecule carriers (M) are attached indirectly to A-X-B by a reactive conjugation group (c_{A}, c_{B}, and c_{M}). In some embodiments, the cargo (e.g., D_{A} and D_{B}) and the macromolecule carriers (M) are attached indirectly to A-X-B by an orthogonally reactive conjugation group (c_{A}, c_{B}, and c_{M}). In some embodiments, c_{A}, c_{B}, and c_{M} each independently comprise an amino acid. In some embodiments, c_{A}, c_{B}, and c_{M} each independently comprise 0-10 amino acids. In some embodiments, c_{A}, c_{B}, and c_{M} each independently comprise 1 amino acid. In some embodiments, c_{A}, c_{B}, and c_{M} each independently comprise 2 amino acids. In some embodiments, c_{A}, c_{B}, and c_{M} each independently comprise 3 amino acids. In some embodiments, c_{A}, c_{B}, and c_{M} each independently comprise 4 amino acids. In some embodiments, c_{A}, c_{B}, and c_{M} each independently comprise 5 amino acids. In some embodiments, c_{A}, c_{B}, and c_{M} each independently comprise 6 amino acids. In some embodiments, c_{A}, c_{B}, and c_{M} each independently comprise 7 amino acids. In some embodiments, c_{A}, c_{B}, and c_{M} each independently comprise 8 amino acids. In some embodiments, c_{A}, c_{B}, and c_{M} each independently comprise 9 amino acids. In some embodiments, c_{A}, c_{B}, and c_{M} each independently comprise 10 amino acids.

In some embodiments, c_{A}, c_{B}, and c_{M} each independently comprise a derivatized amino acid. In some embodiments, multiple cargoes (D) are attached to a derivatized amino acid conjugation group.

In some embodiments, the conjugation group comprises a receptor ligand.

In some embodiments, c_{A}, c_{B}, and c_{M} each independently comprise a naturally-occurring amino acid or a non-naturally-occurring amino acid. In some embodiments, c_{A}, c_{B}, and c_{M} each independently comprise from a D amino acid, a L amino acid, an α-amino acid, a β-amino acid, or a α-amino acid. In some embodiments, c_{A}, c_{B}, and c_{M} each independently comprise any amino acid having a free thiol group, any amino acid containing a free amine group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} each independently comprise D-cysteine, D-glutamate, lysine, and para-4-acetyl L-phenylalanine. In some embodiments, c_{B} comprises any amino acid having a free thiol group. In some embodiments, c_{B} comprises D-cysteine. In some embodiments, c_{A} comprises any amino acid having a N-terminal amine group. In some embodiments, c_{A} comprises D-glutamate. In some embodiments, c_{A} comprises lysine. In some embodiments, c_{M} comprises any amino acid with a side chain capable of forming an oxime or hydrazone bond upon reaction with a hydroxylamine or hydrazine group. In some embodiments, c_{M} comprises para-4-acetyl L-phenylalanine.

In some embodiments, c_{A}, c_{B}, and c_{M} are each independently selected from a naturally-occurring amino acid or a non-naturally-occurring amino acid. In some embodiments, c_{A}, c_{B}, and c_{M} are each independently selected from a D amino acid, a L amino acid, an α-amino acid, a β-amino acid, or a γ-amino acid. In some embodiments, c_{A}, c_{B}, and c_{M} are each independently any amino acid having a free thiol group, any amino acid containing a free amine group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from: D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{A} is lysine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine.

### Cargo (D)

### Therapeutic Agents

Disclosed herein, in certain embodiments, is the use of a selective delivery molecule disclosed herein for delivering a therapeutic agent to a tissue or a plurality of cells. In some embodiments, the therapeutic agent is an anti-inflammatory agent. In some embodiments, the therapeutic agent is an anti-cancer agent. In some embodiments, the selective delivery molecule is used to treat colorectal cancer.

In some embodiments, a D moiety is independently a therapeutic agent. In some embodiments, a D moiety comprises two or more therapeutic agents. In some embodiments, the two or more therapeutic agents are the same therapeutic agent. In some embodiments, the two or more therapeutic agents are different therapeutic agents. In some embodiments, a D moiety comprises 2, 3, 4, 5, 6, 7, 8, 9, 10, 11, 12 or more therapeutic agents. In some embodiments, the therapeutic agent is selected from: a chemotherapeutic agent, a steroid, an immunotherapeutic agent, a targeted therapy, an anti-inflammatory agent, or a combination thereof. In some embodiments, the therapeutic agent is a radiotherapeutic agent. In some embodiments, the therapeutic agent is a cytotoxin.

In some embodiments, the therapeutic agent is a B cell receptor pathway inhibitor. In some embodiments, the therapeutic agent is a CD79A inhibitor, a CD79B inhibitor, a CD19 inhibitor, a Lyn inhibitor, a Syk inhibitor, a PI3K inhibitor, a Blnk inhibitor, a PLCy inhibitor, a PKCD3 inhibitor, or a combination thereof. In some embodiments, the therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacytlase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof. In some embodiments, the therapeutic agent is selected from: chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, bendamustine, chlorambucil, chlormethine, cyclophosphamide, ifosfamide, melphalan, prednimustine, trofosfamide, busulfan, mannosulfan, treosulfan, carboquone, thiotepa, triaziquone, carmustine, fotemustine, lomustine, nimustine, ranimustine, semustine, streptozocin, etoglucid, dacarbazine, mitobronitol, pipobroman, temozolomide, methotrexate, permetrexed, pralatrexate, raltitrexed, cladribine, clofarabine, fludarabine, mercaptopurine, nelarabine, tioguanine, azacitidine, capecitabine, carmofur, cytarabine, decitabine, fluorouracil, gemcitabine, tegafur, vinblastine, vincristine, vindesine, vinflunine, vinorelbine, etoposide, teniposide, demecolcine, docetaxel, paclitaxel, paclitaxel poliglumex, trabectedin, dactinomycin, aclarubicin, daunorubicin, doxorubicin, epirubicin, idarubicin, mitoxantrone, pirarubicin, valrubicin, zorubincin, bleomycin, ixabepilone, mitomycin, plicamycin, carboplatin, cisplatin, oxaliplatin, satraplatin, procarbazine, aminolevulinic acid, efaproxiral, methyl aminolevulinate, porfimer sodium, temoporfin, dasatinib, erlotinib, everolimus, gefitinib, imatinib, lapatinib, nilotinib, pazonanib, sorafenib, sunitinib, temsirolimus, alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat, diethylstilbenol, ethinylestradiol, fosfestrol, polyestradiol phosphate, gestonorone, medroxyprogesterone, megestrol, buserelin, goserelin, leuprorelin, triptorelin, fulvestrant, tamoxifen, toremifene, bicalutamide, flutamide, nilutamide, aminoglutethimide, anastrozole, exemestane, formestane, letrozole, vorozole, abarelix, degarelix, histamine dihydrochloride, mifamurtide, pidotimod, plerixafor, roquinimex, thymopentin, everolimus, gusperimus, leflunomide, mycophenolic acid, sirolimus, ciclosporin, tacrolimus, azathioprine, lenalidomide, methotrexate, thalidomide, iobenguane, ancestim, filgrastim, lenograstim, molgramostim, pegfilgrastim, sargramostim, interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b, aldesleukin, oprelvekin. BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin, abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus, adalimumab, afelimomab, certolizumab pegol, etanercept, golimumab, infliximab, anakinra, basiliximab, canakinumab, daclizumab, mepolizumab, rilonacept, tocilizumab, ustekinumab, ciclosporin, tacrolimus, azathioprine, lenalidomide, methotrexate, thalidomide, adalimumab, alemtuzumab, bevacizumab, cetuximab, certolizumab pegol, eculizumab, efalizumab, gemtuzumab, ibritumomab tiuxetan, muromonab-CD3, natalizumab, panitumumab, ranibizumab, rituximab, tositumomab, trastuzumab, catumaxomab, edrecolomab, ofatumumab, muromab-CD3, afelimomab, golimumab, ibritumomab tiuxetan, abagovomab, adecatumumab, alemtuzumab, anti-CD30 monoclonal antibody Xmab2513, anti-MET monoclonal antibody MetMab, apolizumab, apomab, arcitumomab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab, a syk inhibitor (e.g., R788), enzastaurin, dasatinib, erlotinib, everolimus, gefitinib, imatinib, lapatinib, nilotinib, pazonanib, sorafenib, sunitinib, temsirolimus, an angiogenesis inhibitor (e.g., GT-111, JI-101, R1530), a kinase inhibitors (e.g., AC220, AC480, ACE-041, AMG 900, AP24534, Arry-614, AT7519, AT9283, AV-951, axitinib, AZD 1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919,Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SARI03168, S3333333CH 727965, SGI-1176, SGX523, SNS-314, TAK-593, TAK-901, TK1258, TLN-232, TTP607, XL147, XL228, XL281RO5126766, XL418, XL765), an inhibitor of mitogen-activated protein kinase signaling (e.g., U0126, PD98059, PD 184352, PD0325901, ARRY-142886, SB239063, SP600125, BAY 43-9006, wortmannin, or LY294002), adriamycin, dactinomycin, bleomycin, vinblastine, cisplatin, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, iimofosine, interleukin II (including recombinant interleukin II, or rIL2), interferon alfa-2a, interferon alfa-2b, interferon alfa-n1, interferon alfa-n3, interferon beta-1a, interferon gamma-1b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazoie, nogalamycin, ormaplatin, oxisuran, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. In some embodiments, the therapeutic agent is selected from: 20-epi-1, 25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1,i antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole. CaRest M3, CARN 700, cartilage derived inhibitor, carzelesin, casein kinase inhibitors (ICOS), castanospermine, cecropin B, cetrorelix, chlorlns, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, 9-dioxamycin, diphenyl spiromustine, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-such as for example growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylerie conjugate, raf antagonists, raltitrexed, ramosetron, ras famesyl protein transferase inhibitors, ras inhibitors, ras-GAP inhibitor, retelliptine demethylated, rhenium Re 186 etidronate, rhizoxin, ribozymes. RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen-binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins. UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer, mechloroethamine, cyclophosphamide, chlorambucil, busulfan, carmustine, lomusitne, decarbazine, methotrexate, cytarabine, mercaptopurine, thioguanine, pentostatin, mechloroethamine, cyclophosphamide, chlorambucil, meiphalan, ethylenimine, methylmelamine, hexamethlymelamine, thiotepa, busulfan, carmustine, lomusitne, semustine, streptozocin, decarbazine, fluorouracil, floxouridine, cytarabine, mercaptopurine, thioguanine, pentostatin, erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7. Spongistatin 8, and Spongistatin 9), Cemadotin hydrochloride (also known as LU-103793 and NSC-D-669356), Epothilones (such as Epothilone A. Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide. Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027). LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda). KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Ser.HCI, and RPR-258062A), Vitilevuamide, Tubulysin A, Canadensol, Centaureidin (also known as NSC-106969), T-138067 (Tularik, also known as T-67, TL-138067 and TI-138067), COBRA-I (Parker Hughes Institute, also known as DDE-261 and WHI-261). H10 (Kansas State University), H16 (Kansas State University), Oncocidin A1 (also known as BTO-956 and DIME), DDE-313 (Parker Hughes Institute). Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191). TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin. Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI). Resverastatin phosphate sodium, BPR-OY-007 (National Health Research Institutes), and SSR-250411 (Sanofi).

In some embodiments, the therapeutic agent is an anti-inflammatory agent. In some embodiments, the therapeutic agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent, an alkylating agent, an anti-metabolite, a plant alkaloid, a terpenoids, a topoisomerase inhibitor, an antitumour antibiotic, an antibody, a hormonal therapy, an anti-diabetes agent, a leukotriene inhibitor, or combinations thereof. In some embodiments, the therapeutic agent is selected from: alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI). Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.). Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Comell University). TB-403 (Biolnvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine. AIN457 (Anti IL-17 Monoclonal Antibody, Novartis), theophylline, D2E7 (a human anti-TNF mAb from Knoll Pharmaceuticals), Mepolizumab (Anti-IL-S antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-Ibeta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimethyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca)), [(1R)-3-methyl-1-({(2S)-3-phenyl-2-[(pyrazin-2-ylcarbonyl)amino]propanoyl}amino)butyl boronic acid (Bortezomib), AMG-714, (Anti-IL 15 Human Monoclonal Antibody, Amgen), ABT-874 (Anti IL-12 monoclonal antibody, Abbott Labs). MRA(Tocilizumab, an Anti IL-6 Receptor Monoclonal Antibody, Chugai Pharmaceutical), CAT-354 (a human anti-interleukin-13 monoclonal antibody. Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol, Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin. (Rosiglitazone+glimepiride), Exenatide. Insulin, Sitagliptin. (glipizide and metformin), Repaglinide. Acarbose, Nateglinide, Orlistat, cisplatin; carboplatin: oxaliplatin; mechlorethamine; cyclophosphamide; chlorambucil; vincristine; vinblastine; vinorelbine; vindesine; mercaptopurine: fludarabine: pentostatin; cladribine; 5-fluorouracil (5FU): floxuridine (FUDR); cytosine arabinoside; trimethoprim; pyrimethamine; pemetrexed: paclitaxel: docetaxel; etoposide; teniposide; irinotecan: topotecan: amsacrine: etoposide; etoposide phosphate; teniposide: dactinomycin: doxorubicin; daunorubicin; valrubicine; idarubicine; epirubicin; bleomycin; plicamycin; mitomycin: finasteride; goserelin: aminoglutethimide; anastrozole; letrozole: vorozole; exemestane: 4-androstene-3,6,17-trione (“6-OXO”: 1,4,6-androstatrien-3,17-dione (ATD); formestane; testolactone; fadrozole: A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid: AME103 (Amira); AME803 (Amira); atreleuton: BAY-x-1005 ((R)-(+)-alpha-cyclopentyl-4-(2-quinolinylmethoxy)-Benzeneacetic acid): CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-[1(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy] acetic acid); SA6541 ((R)—S-[[4-(dimethylamino)phenvl]methyl]-N-(3-mercapto-2methyl-1-oxopropyl-L-cycteine); SC-56938 (ethyl-1-[2-[4-(phenylmethyl)phenoxy]ethyl]-4-piperidine-carboxylate): VIA-2291 (Via Pharmaceuticals): WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); doxycycline: or combinations thereof.

In some embodiments, the therapeutic agent contains a radioactive moiety, for example a radioactive isotope such as ²¹¹At, ¹³¹I, ⁹⁰Y, ¹⁸⁶Re, ¹⁸⁸Re, ¹⁵³Sm, ²¹²Bi, ³²P, ⁶⁴Cu radioactive isotopes of Lu, and others.

### Macromolecular Carriers (M)

Polymers are characterized by a distribution of molecular weights, and, as such, the molecular weight, presented herein for polymers, is only an approximate average molecular weight of a distribution of molecular weights of individual polymers. Unless stated otherwise, the molecular weight of a polymeric component will have a typical (i.e., as known in the art) error and standard deviation.

In some embodiments, a carrier modulates plasma half-life of a selective delivery molecule disclosed herein. In some embodiments, a carrier modulates solubility of a selective delivery molecule disclosed herein. In some embodiments, a carrier modulates bio-distribution of a selective delivery molecule disclosed herein.

In some embodiments, a carrier decreases uptake of a selective delivery molecule by non-target cells or tissues. In some embodiments, a carrier decreases uptake of a selective delivery molecule into cartilage. In some embodiments, a carrier decreases uptake of a selective delivery molecule into joints relative to target tissue.

In some embodiments, a carrier increases uptake of a selective delivery molecule by target cells or tissues. In some embodiments, a carrier decreases uptake of a selective delivery molecule into the liver relative to target tissue. In some embodiments, a carrier decreases uptake of a selective delivery molecule into kidneys. In some embodiments, a carrier enhances uptake into cancer tissue. In some embodiments, a carrier enhances uptake into lymphatic channels and/or lymph nodes.

In some embodiments, a carrier increases plasma half-life by reducing glomerular filtration. In some embodiments, a carrier modulates plasma half-life by increasing or decreases metabolism or protease degradation. In some embodiments, a carrier increases tumor uptake due to enhanced permeability and retention (EPR) of tumor vasculature. In some embodiments, a carrier increases the aqueous solubility of selective delivery molecule.

In some embodiments, any M is independently directly or indirectly (e.g., via c₁) bound to A, B, or X. In some embodiments, any M is independently bound to A at the n-terminal poly glutamate. In some embodiments, any M is independently bound to A (or, the n-terminal poly glutamate) by a covalent linkage. In some embodiments, any M is independently bound to B at the c-terminal polyarginine. In some embodiments, any M is independently bound to B (or, the c-terminal polyarginine) by a covalent linkage. In some embodiments, any M is independently directly or indirectly bound to linkers between X and A, X and B, B and C/N terminus, and A and C/N terminus. In some embodiments, the covalent linkage comprises an ether bond, thioether bond, amine bond, amide bond, oxime bond, carbon-carbon bond, carbon-nitrogen bond, carbon-oxygen bond, or carbon-sulfur bond.

In some embodiments, M is selected from a protein, a synthetic or natural polymer, or a dendrimer. In some embodiments, M is selected from dextran, a PEG polymer (e.g., a PEG polymer having an average molecular weight of approximately 0.5 kDa (PEG 0.5 kDa), approximately 1 kDa (PEG 1 kDa), approximately 2 kDa (PEG 2 kDa), approximately approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa))), albumin, or a combination thereof. In some embodiments, M is a PEG polymer.

In some embodiments, the size of M is between about 500 kDa and about 70 kDa.

In some embodiments, the selective delivery molecule is conjugated to albumin. In certain instances, albumin is excluded from the glomerular filtrate under normal physiological conditions. In some embodiments, the selective delivery molecule comprises a reactive group such as maleimide that can form a covalent conjugate with albumin. A selective delivery molecule comprising albumin results in enhanced accumulation of cleaved selective delivery molecules in tumors in a cleavage dependent manner. In some embodiments, albumin conjugates have good pharmacokinetic properties.

In some embodiments, the selective delivery molecule is conjugated to PEG polymers. In some embodiments, the selective delivery molecule is conjugated to PEG polymers having an average molecular weight of approximately 0.5 kDa (PEG 0.5 kDa). In some embodiments, the selective delivery molecule is conjugated to PEG polymers having an average molecular weight of approximately 1 kDa (PEG 1 kDa). In some embodiments, the selective delivery molecule is conjugated to PEG polymers having an average molecular weight of approximately 2 kDa (PEG 2 kDa). In some embodiments, the selective delivery molecule is conjugated to PEG polymers having an average molecular weight of approximately 3 kDa (PEG 3 kDa). In some embodiments, the selective delivery molecule is conjugated to PEG polymers having an average molecular weight of approximately 4 kDa (PEG 4 kDa). In some embodiments, the selective delivery molecule is conjugated to PEG polymers having an average molecular weight of approximately 5 kDa (PEG 5 kDa). In some embodiments, the selective delivery molecule is conjugated to PEG polymers having an average molecular weight of approximately 10 kDa (PEG 10 kDa). In some embodiments, the selective delivery molecule is conjugated PEG polymers having an average molecular weight of approximately 12 kDa (PEG 12 kDa). In some embodiments, the selective delivery molecule is conjugated to PEG polymers having an average molecular weight of approximately 15 kDa (PEG 15 kDa). In some embodiments, selective delivery molecule is conjugated to PEG polymers having an average molecular weight of approximately 20 kDa (PEG 20 kDa). In some embodiments, selective delivery molecule is conjugated to PEG polymers having an average molecular weight of approximately 30 kDa (PEG 30 kDa). In some embodiments, selective delivery molecules conjugated to PEG30 kDa had a longer half-life as compared to free peptides. In some embodiments, selective delivery molecules are conjugated to PEG polymers having an average molecular weight of between about 20 to about 40 kDa which have hepatic and renal clearance.

The PEG groups are polydisperse and have a distribution of molecular weights. Thus, any characterization of a PEG group should be interpreted in light of the polydispersity of PEG, unless otherwise stated.

In some embodiments, the selective delivery molecule is conjugated to a dextran. In some embodiments, the selective delivery molecule is conjugated to a dextran having a molecular weight of approximately 70 kDa. In some embodiments, dextran conjugates, being a mixture of molecular weights, are difficult to synthesize and purify reproducibly.

In some embodiments, the selective delivery molecule is conjugated to streptavidin.

In some embodiments, the selective delivery molecule is conjugated to a fifth generation PAMAM dendrimer.

In some embodiments, a carrier is capped. In some embodiments, capping a carrier improves the pharmacokinetics and reduces cytotoxicity of a carrier by adding hydrophilicity. In some embodiments, the cap is selected from: Acetyl, succinyl, 3-hydroxypropionyl, 2-sulfobenzoyl, glycidyl, PEG-2, PEG-4, PEG-8 and PEG-12.

### Portion X (Extracellular Cleavable Linkers)

In some embodiments, X is a linker consisting of one or more amino acids is used to join peptide sequence A (i.e., the sequence designed to inhibit the delivery action of peptide B) and peptide sequence B. Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In live cells, an intact selective delivery molecule disclosed herein may not be able to enter the cell because of the presence of portion of A. Thus, a strictly intracellular process for cleaving X would be ineffective to cleave X in healthy cells since portion of A, preventing uptake into cells, would not be effectively cleaved by intracellular enzymes in healthy cells since it would not be taken up and would not gain access to such intracellular enzymes. However, where a cell is injured or diseased (e.g., cancerous cells, hypoxic cells, ischemic cells, apoptotic cells, necrotic cells) such intracellular enzymes leak out of the cell and cleavage of A would occur, allowing entry of portion of B and/or cargo into the cell, effecting targeted delivery of portion of B and/or cargo D to neighboring cells. In some embodiments, X is cleaved in the extracellular space.

In some embodiments, the fact that capillaries are often leaky around tumors and other trauma sites enhances the ability of high molecular weight molecules (e.g., molecular weight of about 30 kDa or more) to reach the interstitial compartment. In some embodiments, cells that do not express the relevant protease but that are immediately adjacent to expressing cells pick up cargo from a selective delivery molecule because linkage of a X linker is typically extracellular.

In some embodiments, such bystander targeting is beneficial in the treatment of tumors because of the heterogeneity of cell phenotypes and the wish to eliminate as high a percentage of suspicious cells as possible.

In some embodiments, X is a cleavable linker.

In some embodiments, the X linker is flexible. In some embodiments, the linker is rigid.

In some embodiments, the X linker comprises a linear structure. In some embodiments, the X linker comprises a non-linear structure. In some embodiments, the X linker comprises a branched structure. In some embodiments, the X linker comprises a cyclic structure.

In some embodiments, X is about 5 to about 30 atoms in length. In some embodiments, X is about 6 atoms in length. In some embodiments, X is about 8 atoms in length. In some embodiments, X is about 10 atoms in length. In some embodiments, X is about 12 atoms in length. In some embodiments, X is about 14 atoms in length. In some embodiments, X is about 16 atoms in length. In some embodiments, X is about 18 atoms in length. In some embodiments, X is about 20 atoms in length. In some embodiments, X is about 25 atoms in length. In some embodiments. X is about 30 atoms in length.

In some embodiments, the linker binds peptide portion of A (i.e., the peptide sequence which prevents cellular uptake) to peptide portion of B (i.e., the delivery sequence) by a covalent linkage. In some embodiments, the covalent linkage comprises an ether bond, thioether bond, amine bond, amide bond, oxime bond, hydrazone bond, carbon-carbon bond, carbon-nitrogen bond, carbon-oxygen bond, or carbon-sulfur bond.

In some embodiments, X comprises a peptide linkage. The peptide linkage comprises L-amino acids and/or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a X linker is designed for cleavage in the presence of particular conditions or in a particular environment. In preferred embodiments, a X linker is cleavable under physiological conditions. Cleavage of such a X linker may, for example, be enhanced or may be affected by particular pathological signals or a particular environment related to cells in which cargo delivery is desired. The design of a X linker for cleavage by specific conditions, such as by a specific enzyme, allows the targeting of cellular uptake to a specific location where such conditions obtain. Thus, one important way that selective delivery molecules provide specific targeting of cellular uptake to desired cells, tissues, or regions is by the design of the linker portion X to be cleaved by conditions near such targeted cells, tissues, or regions.

In some embodiments, X is a pH-sensitive linker. In some embodiments. X is cleaved under basic pH conditions. In some embodiments, X is cleaved under acidic pH conditions. In some embodiments, X is cleaved by a protease, a matrix metalloproteinase, or a combination thereof. In some embodiments, X is cleaved by a reducing agent.

In some embodiments, X is cleaved by an MMP. The hydrolytic activity of matrix metalloproteinases (MMPs) has been implicated in the invasive migration of metastatic tumor cells. In certain instances, MMPs are found near sites of inflammation. In certain instances, MMPs are found near sites of stroke (i.e., a disorder characterized by brain damage following a decrease in blood flow). Thus, uptake of molecules having features of the invention are able to direct cellular uptake of cargo (at least one D moiety) to specific cells, tissues, or regions having active MMPs in the extracellular environment. In some embodiments, a X linker that includes the amino-acid sequences PLG-C(Me)-AG (SEQ ID NO: 1), PLGLAG (SEQ ID NO: 2) which are cleaved by the metalloproteinase enzymes MMP-2, MMP-9, or MMP-7 (MMPs involved in cancer and inflammation).

In some embodiments, X is cleaved by proteolytic enzymes or reducing environment, as may be found near cancerous cells. Such an environment, or such enzymes, are typically not found near normal cells.

In some embodiments, X is cleaved by serine proteases including but not limited to thrombin.

In some embodiments, X is cleaved in or near tissues suffering from hypoxia. In some embodiments, cleavage in or near hypoxic tissues enables targeting of cancer cells and cancerous tissues, infarct regions, and other hypoxic regions. In some embodiments, X comprises a disulfide bond. In some embodiments, a linker comprising a disulfide bond is preferentially cleaved in hypoxic regions and so targets cargo delivery to cells in such a region. Hypoxia is thought to cause cancer cells to become more resistant to radiation and chemotherapy, and also to initiate angiogenesis. In a hypoxic environment in the presence of, for example, leaky or necrotic cells, free thiols and other reducing agents become available extracellularly, while the O₂ that normally keeps the extracellular environment oxidizing is by definition depleted. In some embodiments, this shift in the redox balance promotes reduction and cleavage of a disulfide bond within a X linker. In addition to disulfide linkages which take advantage of thiol-disulfide equilibria, linkages including quinones that fall apart when reduced to hydroquinones are used in a X linker designed to be cleaved in a hypoxic environment.

In some embodiments, X is cleaved in a necrotic environment. Necrosis often leads to the release of enzymes or other cell contents that may be used to trigger cleavage of a X linker. In some embodiments, cleavage of X by necrotic enzymes (e.g., by calpains) allows cargo to be taken up by diseased cells and by neighboring cells that had not yet become fully leaky.

In some embodiments, X is an acid-labile linker. In some embodiments, X comprises an acetal or vinyl ether linkage. Acidosis is observed in sites of damaged or hypoxic tissue, due to the Warburg shift from oxidative phosphorylation to anaerobic glycolysis and lactic acid production. In some embodiments, acidosis is used as a trigger of cargo uptake by replacing some of the arginines within B by histidines, which only become cationic below pH 7.

It will be understood that a linker X disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

In some embodiments, the linker X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9). PPRSFL (SEQ ID NO: 10), RLQLKL (SEQ ID NO: 11), and RLQLK(Ac) (SEQ ID NO: 12). In some embodiments, the linker X comprises the amino acid sequence PLGLAG (SEQ ID NO: 2). In some embodiments, the linker X comprises the amino acid sequence PLG-C(me)-AG (SEQ ID NO: 1). In some embodiments, the linker X comprises the amino acid sequence PLGxAG (SEQ ID NO: 20), wherein x is any amino acid (naturally-occurring or non-naturally occurring). In some embodiments, the linker X comprises the amino acid sequence RPLALWRS (SEQ ID NO: 7). In some embodiments, the linker X comprises the amino acid sequence ESPAYYTA (SEQ ID NO: 8). In some embodiments, the linker X comprises the amino acid sequence DPRSFL (SEQ ID NO: 9). In some embodiments, the linker X comprises the amino acid sequence PPRSFL (SEQ ID NO: 10).

In some embodiments, the linker X comprises the amino acid sequence RLQLKL (SEQ ID NO: 11). In some embodiments, the linker X comprises the amino acid sequence RLQLK(Ac) (SEQ ID NO: 12).

In some embodiments, the linker X comprises a peptide selected from: PR(S/T)(L/I)(S/T), where the letters in parentheses indicate that either one of the indicated amino acids may be at that position in the sequence); GGAANLVRGG (SEQ ID NO: 21); SGRIGFLRTA (SEQ ID NO: 22); SGRSA (SEQ ID NO: 23); GFLG (SEQ ID NO: 24): ALAL (SEQ ID NO: 25): FK; PIC(Et)F-F (SEQ ID NO: 26), where C(Et) indicates S-ethylcysteine (a cysteine with an ethyl group attached to the thiol) and the “-” indicates the typical cleavage site in this and subsequent sequences); GGPRGLPG (SEQ ID NO: 27); HSSKLQ (SEQ ID NO: 28); LVLA-SSSFGY (SEQ ID NO: 29); GVSQNY-PIVG (SEQ ID NO: 30); GVVQA-SCRLA (SEQ ID NO: 31): f(Pip)R-S, where “f” indicates D-phenylalanine and “Pip” indicates piperidine-2-carboxylic acid (pipecolinic acid, a proline analog having a six-membered ring); DEVD (SEQ ID NO: 32); GWEHDG (SEQ ID NO: 33); RPLALWRS (SEQ ID NO: 7), or a combination thereof.

In some embodiments, X is cleaved under hypoxic conditions. In some embodiments, X comprises a disulfide linkage. In some embodiments, X comprises a quinine.

In some embodiments, X is cleaved under necrotic conditions. In some embodiments, X comprises a molecule cleavable by a calpain.

In some embodiments, X comprises 6-aminohexanoyl, 5-(amino)-3-oxopentanoyl, or a combination thereof. In some embodiments, X comprises a disulfide linkage.

In some embodiments, the linker is an alkyl. In some embodiments, the linker is heteroalkyl.

In some embodiments, the linker is an alkylene. In some embodiments, the linker is an alkenylene. In some embodiments, the linker is an alkynylene. In some embodiments, the linker is a heteroalkylene.

In some embodiments, a selective delivery molecules disclosed herein comprises a single of linker. Use of a single mechanism to mediate uptake of both imaging and therapeutic cargoes is particularly valuable, because imaging with noninjurious tracer quantities can be used to test whether a subsequent therapeutic dose is likely to concentrate correctly in the target tissue.

In some embodiments, a selective delivery molecules disclosed herein comprises a plurality of linkers. Where a selective delivery molecule disclosed herein includes multiple X linkages, separation of portion of A from the other portions of the molecule requires cleavage of all X linkages. Cleavage of multiple X linkers may be simultaneous or sequential. Multiple X linkages may include X linkages having different specificities, so that separation of portion of A from the other portions of the molecule requires that more than one condition or environment (“extracellular signals”) be encountered by the molecule. Cleavage of multiple X linkers thus serves as a detector of combinations of such extracellular signals. For example, a selective delivery molecule may include two linker portions Xa and Xb connecting basic portion of B with acidic portion of A. Both X linkers a and Xb must be cleaved before acidic portion of A is separated from basic portion of B allowing entry of portion of B and cargo moiety C (if any) to enter a cell. It will be understood that a linker region may link to either a basic portion of B or a cargo moiety C independently of another linker that may be present, and that, where desired, more than two linker regions X may be included.

Combinations of two or more X linkers may be used to further modulate the targeting and delivery of molecules to desired cells, tissue or regions. Combinations of extracellular signals are used to widen or narrow the specificity of the cleavage of X linkers if desired. Where multiple X linkers are linked in parallel, the specificity of cleavage is narrowed, since each X linker must be cleaved before portion of A may separate from the remainder of the molecule.

Where multiple X linkers are linked in series, the specificity of cleavage is broadened, since cleavage of any one X linker allows separation of portion of A from the remainder of the molecule. For example, in order to detect either a protease OR hypoxia (i.e., to cleave X in the presence of either protease or hypoxia), a X linker is designed to place the protease-sensitive and reduction-sensitive sites in tandem, so that cleavage of either would suffice to allow separation of the acidic portion of A. Alternatively, in order to detect the presence of both a protease AND hypoxia (i.e., to cleave X in the presence of both protease and hypoxia but not in the presence of only one alone), a X linker is designed to place the protease sensitive site between at least one pair of cysteines that are disulfide-bonded to each other. In that case, both protease cleavage and disulfide reduction are required in order to allow separation of portion of A.

### Portion Y Linker (Intracellular Cleavable Linkers)

In some embodiments, Y is a linker consisting of one or more amino acids is used to join Cargo (D) to the remainder of the SDM. In some embodiments, Y is a linker consisting of one or more amino acids is used to join Cargo (D) to portion B. Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In some embodiments, the linker binds cargo portion of D to peptide portion of B (i.e., the delivery sequence) by a covalent linkage. In some embodiments, the covalent linkage comprises an ether bond, thioether bond, amine bond, amide bond, oxime bond, hydrazone bond, carbon-carbon bond, carbon-nitrogen bond, carbon-oxygen bond, or carbon-sulfur bond.

In some embodiments, the Y linker is flexible. In some embodiments, the Y linker is rigid. In some embodiments, the Y linker comprises a linear structure. In some embodiments, the Y linker comprises a non-linear structure. In some embodiments, the Y linker comprises a branched structure. In some embodiments, the linker comprises a cyclic structure.

In some embodiments, Y linker comprises a peptide linkage. The peptide linkage comprises L-amino acids and/or D-amino acids. In embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a Y linker is designed for cleavage in the presence of particular conditions or in a particular environment. In some embodiments, a Y linker is cleavable by an intracellular protease. In some embodiments, Y is cleavable by an intracellular protease. In some embodiments, a Y linker is cleavable by a lysosomal protease. In some embodiments, the intracellular protease is a cysteine protease. In some embodiments, the intracellular protease is an aspartyl protease. In some embodiments, the intracellular protease is a serine protease. In some embodiments, the cysteine protease is a caspase, a cathepsin, calpain, papain or a legumain. In some embodiments, the intracellular protease is an initiator caspase. In some embodiments, the intracellular protease is an effector caspase. In some embodiments, the Y linker is cleavable by a protease selected from among cathepsin B, cathepsin L, cathepsin H, cathepsin K, cathepsin W, cathepsin C, cathepsin F, cathepsin V, cathepsin X, cathepsin S, cathepsin D, cathepsin G, HCP-1, HCP-2, dipeptidyl-peptidase I, MEROPS C13, CED-3 peptidase, caspase 2, caspase 3, caspase 6, caspase 7, caspase 8, caspase 9, caspase 10, caspase 11; caspase 12, caspase 13, and caspase 14. In some embodiments, the Y linker is cleavable by a protease selected from among cathepsin B, cathepsin L, caspase 3, caspase 7, caspase 8, and caspase 9. In some embodiments, a Y linker is cleavable by Cathepsin B a dipeptidyl carboxypeptidase. In some embodiments the linker has a lysine, citrulline, or arginine residue at the P1 position and a large hydrophobic residue at the P1′ position.

In some embodiments, the Y linker comprises an acid sensitive chemical linker. In some embodiments, acid sensitive chemical linker is hydrazone or a derivative thereof. In some embodiments, a Y linker comprises a self-immolative spacer. In some embodiments, the self-immolative spacer is of sufficient length to prevent the occurrence of steric hindrance between the B portion of the SDM and the therapeutic cargo. In some embodiments, Y comprises a p-aminobenzyl alcohol (PABOH) spacer or a derivative thereof. In some embodiments, Y comprises a p-aminobenzyl carbonyl (PABC) spacer or a derivative thereof. In some embodiments, Y comprises a branched bis(hydroxymethyl)styrene (BHMS) spacer or a derivative thereof. In some embodiments, Y comprises a 2-aminoimidazol-5-methanol derivative or an ortho or para-aminobenzylacetal spacer. In some embodiments Y comprises 2,6-bishydroxymethyl-p-cresol or hemithioaminal derivatives.

In some embodiments, the Y linker comprises the lysosomally cleavable peptide. In some embodiments, the Y linker comprises the lysosomally cleavable dipeptide Phe-Arg. In some embodiments, the Y linker comprises the lysosomally cleavable dipeptide Phe-Lys. In some embodiments, the Y linker comprises the lysosomally cleavable dipeptide Val-Cit (1-citrulline). In some embodiments, the Y linker comprises the lysosomally cleavable tetrapeptide Gly-Phe-Leu-Gly (SEQ ID NO: 24). In some embodiments, the Y linker comprises the lysosomally cleavable tetrapeptide Ala-Leu-Ala-Leu (SEQ ID NO: 25).

In some embodiments, the Y linker comprises the lysosomally cleavable peptide and a self-immolative spacer.

In some embodiments, Y is a pH-sensitive linker. In some embodiments, Y is cleaved under acidic pH conditions. In some embodiments, Y is cleaved under acidic pH conditions of the lysosome.

It will be understood that a Y linker disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

### Imaging Agents

In some embodiments, an SDM provided herein is conjugated to an imaging agent. In some embodiments, the imaging agent is conjugated to portion of A, portion of B or both portions A and B. In some embodiments, the imaging agent is conjugated to the target ligand.

In some embodiments, an imaging agent is a dye. In some embodiments, an imaging agent is a fluorescent moiety. In some embodiments, a fluorescent moiety is selected from: a fluorescent protein, a fluorescent peptide, a fluorescent dye, a fluorescent material or a combination thereof.

All fluorescent moieties are encompassed within the term “fluorescent moiety.” Specific of fluorescent moieties given herein are illustrative and are not meant to limit the fluorescent moieties for use with the targeting molecules disclosed herein.

of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins): aromatic amines (e.g., dansyl: squarate dyes); benzofurans; fluorescent cyanines: indocarbocyanines: carbazoles: dicyanomethylene pyranes: polymethine; oxabenzanthrane; xanthene; pyrylium; carbostyl: perylene; acridone: quinacridone; rubrene; anthracene; coronene; phenanthrecene; pyrene: butadiene; stilbene; porphyrin: pthalocyanine: lanthanide metal chelate complexes: rare-earth metal chelate complexes, and derivatives of such dyes.

of fluorescein dyes include, but are not limited to, 5-carboxyfluorescein, fluorescein-5-isothiocyanate, fluorescein-6-isothiocyanate and 6-carboxyfluorescein.

of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the trade name of TEXAS RED®).

of cyanine dyes include, but are not limited to, Cy3, Cy3B, Cy3.5, Cy5, Cy5.5, Cy7, IRDYE680, Alexa Fluor 750, IRDye800CW, ICG.

of fluorescent peptides include GFP (Green Fluorescent Protein) or derivatives of GFP (e.g., EBFP. EBFP2, Azurite, mKalamal, ECFP, Cerulean, CyPet, YFP, Citrine, Venus, YPet).

Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultiplier, etc.

In some embodiments, the imaging agent is labeled with a positron-emitting isotope (e.g., ¹⁸F) for positron emission tomography (PET), gamma-ray isotope (e.g., ⁹⁹Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the imaging agent is labeled with: a gadolinium chelate, an iron oxide particle, a super paramagnetic iron oxide particle, an ultra small paramagnetic particle, a manganese chelate or gallium containing agent.

of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the imaging agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indiuim chelate.

of Tc chelates include, but are not limited to HYNIC. DTPA, and DOTA.

In some embodiments, the imaging agent contains a radioactive moiety, for example a radioactive isotope such as ²¹¹At, ¹³¹I, ⁹⁰Y, ¹⁸⁶Re, ¹⁸⁸Re, ¹⁵³Sm, ²¹²Bi, ³²P, ⁶⁴Cu radioactive isotopes of Lu, and others.

In some embodiments, a selective delivery molecule according to Formulas I-VI comprising an imaging agent is employed in guided surgery. In some embodiments, the selective delivery molecule preferentially localized to cancerous, or other undesirable tissues (i.e. necrotic tissues). In some embodiments, a selective delivery molecule according to Formula I comprising an imaging agent is employed in a guided surgery to remove colorectal cancer. In some embodiments, guided surgery employing the selective delivery molecule allows a surgeon to excise as little healthy (i.e., non-cancerous) tissue as possible. In some embodiments, guided surgery employing the selective delivery molecule allows a surgeon to visualize and excise more cancerous tissue than the surgeon would have been able to excise without the presence of the selective delivery molecule. In some embodiments, the surgery is fluorescence-guided surgery.

### Exemplary Selective Delivery Molecules

In some embodiments, the selective delivery molecule comprises a structure selected from SDM-101, SDM-102, SDM-103, SDM-104, SDM-105, SDM-106, SDM-107, SDM-108, SDM-109, SDM-110, SDM-111, SDM-112, SDM-113, SDM-114, SDM-115, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153. In some embodiments, the selective delivery molecule is a derivative of SDM-101, SDM-102, SDM-103, SDM-104, SDM-105, SDM-106, SDM-107, SDM-108, SDM-109, SDM-10, SDM-11, SDM-112, SDM-13, SDM-14, SDM-15, SDM-116, SDM-117, SDM-118, SDM-119, SDM-120, SDM-121, SDM-122, SDM-123, SDM-124, SDM-125, SDM-126, SDM-127, SDM-128, SDM-129, SDM-130, SDM-131, SDM-132, SDM-133, SDM-134, SDM-135, SDM-136, SDM-137, SDM-138, SDM-139, SDM-140, SDM-141, SDM-142, SDM-143, SDM-144, SDM-145, SDM-146, SDM-147, SDM-148, SDM-149, SDM-150, SDM-151, SDM-152, and SDM-153. In some embodiments, the selective delivery molecule the derivative comprises an imaging agent. In some embodiments, the selective delivery molecule the derivative comprises an additional therapeutic agent.

In some embodiments, the selective delivery molecule comprises a structure selected from: SDM-1, SDM-2, SDM-3, SDM-4, SDM-5, SDM-6, SDM-7, SDM-8, SDM-9, SDM-10, SDM-11, SDM-12, SDM-13, SDM-14, SDM-15, SDM-16, SDM-17, SDM-18, SDM-19, SDM-20, SDM-21, SDM-22, SDM-23, SDM-24, SDM-25, SDM-26, SDM-27, SDM-28, SDM-29, SDM-30, SDM-31, SDM-32, SDM-33, SDM-34, SDM-35, SDM-36, SDM-37, SDM-38, SDM-39, SDM-40, SDM-41, SDM-42, SDM-43, SDM-44, SDM-45, SDM-46, SDM-47, SDM-48, SDM-49, SDM-50, SDM-51, SDM-52, SDM-53, SDM-54, SDM-55, SDM-56, SDM-57, SDM-58, SDM-59, SDM-60, and SDM-61 (see International PCT Pub. No. WO2013/019681). In some embodiments, the selective delivery molecule comprises a structure selected from: SDM-14, SDM-15, SDM-23, SDM-24, SDM-25, SDM-26, SDM-27, SDM-32, or SDM-35. In certain embodiments, the selective delivery molecule is derived from Peptide P-1, P-2, P-3, P-4, P-5, P-6, P-7, P-8, P-9, P-10, P-11, P-12, P-13, P-14, P-15, P-16, P-17, P-18, P-19, P-20, P-21, P-21, or P-3,

### FURTHER MODIFICATIONS

In some embodiments, the antibody-conjugated SDMs described herein are optionally conjugated to high molecular weight molecules that increase the multivalency and avidity of labeling. In some embodiments, the high molecular weight molecules are water-soluble polymers.

of suitable water-soluble polymers include, but are not limited to, peptides, saccharides, poly(vinyls), poly(ethers), poly(amines), poly(carboxylic acids) and the like. In some embodiments, the water-soluble polymer is dextran, polyethylene glycol (PEG), polyoxyalkylene, polysialic acid, starch, or hydroxyethyl starch. Any suitable method is used to conjugate peptides to water-soluble polymers (see Hermanson G., Bioconjugate Techniques 2^{nd} Ed., Academic Press, Inc. 2008).

### Pharmaceutical Compositions

Disclosed herein, in certain embodiments, are pharmaceutical compositions comprising any of SDMs as disclosed herein. In some embodiments, the pharmaceutical compositions comprising an SDM comprises an SDM of any of Formulas I-VI and a pharmaceutically acceptable carrier.

Disclosed herein, in certain embodiments, are pharmaceutical compositions comprising any of the antibody-conjugated SDMs as disclosed herein. In some embodiments, the pharmaceutical compositions comprising an antibody-conjugated SDM comprises a targeting antibody conjugated to an SDM of any of Formulas I-VI and a pharmaceutically acceptable carrier.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A, and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a pharmaceutical composition disclosed herein further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In certain embodiments, a pharmaceutical composition disclosed herein is administered to a subject by any suitable administration route, including but not limited to, parenteral (intravenous, subcutaneous, intraperitoneal, intramuscular, intravascular, intrathecal, intravitreal, infusion, or local) administration.

Formulations suitable for intramuscular, subcutaneous, peritumoral, or intravenous injection include physiologically acceptable sterile aqueous or non-aqueous solutions, dispersions, suspensions or emulsions, and sterile powders for reconstitution into sterile injectable solutions or dispersions. of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

Methods of Use

The SDMs of Formulas I-VI and carrier-conjugated SDMs comprising a targeting ligand, e.g. an antibody, allow the targeted delivery of therapeutic agents and/or imaging agents to specific cells and/or tissues. The molecules comprise a basic peptide sequence (B) which is designed to be transported across a cellular membrane, an acidic peptide sequence (A) which inhibits uptake of peptide B into cells, a linker X which is cleavable under specific conditions, cargo moieties (at least D_{A} and D_{B}) bound to peptides A and B, or X and a macromolecular carrier. In some embodiments, cleavage of the linker X linker frees peptide B from peptide A and allows the transport of peptide B (and any cargo attached thereto) across a cellular membrane. In some embodiments, the selective delivery molecules of Formulas I-IV enable targeted delivery of one or more cargoes (e.g., therapeutic agents or imaging agents) to a cell tissue.

Disclosed herein, in certain embodiments, are methods of delivering cargo to a tissue of interest, comprising contacting the tissue of interest with an SDM of any of Formulas I-VI.

Disclosed herein, in certain embodiments, are methods of delivering cargo to a tissue of interest, comprising contacting the tissue of interest with an antibody-conjugated SDM comprising a targeting antibody conjugated to an SDM of any of Formulas I-VI.

### Tissue of Interest

In some embodiments, the tissue of interest is cancerous tissue (or, cancer). In some embodiments, the cancerous tissue is: breast cancer tissue, colon cancer tissue, squamous cell carcinoma tissue, prostate cancer tissue, melanoma tissue, or thyroid cancer tissue. In some embodiments, the cancerous tissue is breast cancer tissue. In some embodiments, the cancerous tissue is colon cancer tissue.

In some embodiments, the tissue of interest is an inflamed tissue. In some embodiments, some embodiments, the inflamed tissue is the result if acute or chronic inflammation. In some embodiments, the inflamed tissue is caused by an inflammatory disease is or is associated with an inflammatory disease. In some embodiments, the inflamed tissue is caused by an inflammatory disease is or is associated with rheumatoid arthritis, osteoarthritis, inflammatory bowel disease, Crohn's disease, ulcerative colitis, sepsis, erythema nodosum leprosum, multiple sclerosis, psoriasis, systemic lupus erythematosis, type I diabetes, atherosclerosis, encephalomyelitis, Alzheimer's disease, stroke, traumatic brain injury, Parkinson's disease or septic shock.

### Therapeutic Uses

The SDMs of Formulas I-VI and carrier-conjugated SDMs comprising a targeting ligand. e.g. an antibody, allow the targeted delivery of therapeutic agents to specific cells and/or tissues (e.g., cancerous tissues). The molecules comprise a basic peptide sequence (B) which is designed to be transported across a cellular membrane, an acidic peptide sequence (A) which inhibits uptake of peptide B into cells, a linker X which is cleavable under specific conditions, therapeutic agents bound to peptides A and B, or X and a macromolecular carrier. In some embodiments, cleavage of the linker X linker frees peptide B from peptide A and allows the transport of peptide B (and any therapeutic agents attached thereto) across a cellular membrane. In some embodiments, the SDMs of Formulas I-VI and carrier-conjugated SDMs comprising a targeting ligand, e.g. an antibody, enable targeted delivery of one or more therapeutic agents to a cell or tissue. In some embodiments, targeted delivery of a therapeutic agent to a cell or tissue enables a medical professional to treat a specific tissue.

In some embodiments, targeted delivery of a therapeutic agent to a cell or tissue enables a medical professional to treat a specific tissue (e.g., cancerous tissue). In some embodiments, targeted delivery of a therapeutic agent to a cell or tissue decreases the dosage of the therapeutic agent. In some embodiments, targeted delivery of a therapeutic agent to a cell or tissue decreases contact of the therapeutic agent with healthy tissue. In some embodiments, targeted delivery of a therapeutic agent to a cell or tissue decreases unwanted side-effects arising from use of high concentrations of a therapeutic agent or contact. In some embodiments, targeted delivery of a therapeutic agent to a cell or tissue decreases unwanted side-effects arising from contact between the therapeutic agent and healthy tissue.

In some embodiments, an SDM of any of Formulas-VI or a carrier-conjugated SDMs comprising a targeting ligand, e.g. an antibody, is employed for the treatment of cancer.

In some embodiments, the cancer is AIDS-related cancers (e.g., AIDS-related lymphoma), anal cancer, basal cell carcinoma, bile duct cancer (e.g., extrahepatic), bladder cancer, bone cancer, (osteosarcoma and malignant fibrous histiocytoma), breast cancer, cervical cancer, colon cancer, colorectal cancer, endometrial cancer (e.g., uterine cancer), ependymoma, esophageal cancer, eye cancer (e.g., intraocular melanoma and retinoblastoma), gastric (stomach) cancer, germ cell tumor, (e.g., extracranial, extragonadal, ovarian), head and neck cancer, leukemia lip and oral cavity cancer, liver cancer, lung cancer (e.g., small cell lung cancer, non-small cell lung cancer, adenocarcinoma of the lung, and squamous carcinoma of the lung), ovarian cancer, pancreatic cancer, pituitary tumor, prostate cancer, renal cancer, skin cancer, small intestine cancer, squamous cell cancer, testicular cancer, throat cancer, thyroid cancer, urethral cancer, and post-transplant lymphoproliferative disorder (PTLD).

In some embodiments, the cancer is a lymphoid cancer (e.g., lymphoma).

In some embodiments, the cancer is a B-cell cancer. In some embodiments, the cancer is precursor B-cell cancers (e.g., precursor B-lymphoblastic leukemia/lymphoma) and peripheral B-cell cancers (e.g., B-cell chronic lymphocytic leukemia/prolymphocytic leukemia/small lymphocytic lymphoma (small lymphocytic (SL) NHL), lymphoplasmacytoid lymphomaimmunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma. Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the cancer is a T-cell and/or putative NK-cell cancer. In some embodiments, the cancer is precursor T-cell cancer (precursor T-lymphoblastic lymphoma/leukemia) and peripheral T-cell and NK-cell cancers (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic yS T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the cancer is Hodgkin's disease.

In some embodiments, the cancer is leukemia. In some embodiments, the cancer is chronic myelocytic I (granulocytic) leukemia, chronic myelogenous, and chronic lymphocytic leukemia (CLL), acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia).

In some embodiments, the cancer is a liquid tumor or plasmacytoma. In some embodiments, the cancer is extramedullary plasmacytoma, a solitary myeloma, and multiple myeloma. In some embodiments, the plasmacytoma is multiple myeloma.

In some embodiments, the cancer is lung cancer.

In some embodiments, the cancer is prostate cancer. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is stage A prostate cancer (the cancer cannot be felt during a rectal exam). In some embodiments, the prostate cancer is stage B prostate cancer (i.e., the tumor involves more tissue within the prostate, it can be felt during a rectal exam, or it is found with a biopsy that is done because of a high PSA level). In some embodiments, the prostate cancer is stage C prostate cancer (i.e., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy. In some embodiments, the prostate cancer is substantially refractory to hormone therapy.

In some embodiments, the prostate cancer is refractory to chemotherapy. In some embodiments, the prostate cancer is metastatic prostate cancer. In some embodiments, the individual is a human who has a gene, genetic mutation, or polymorphism associated with prostate cancer (e.g., RNASEL/HPC1, ELAC2/HPC2, SR-A/MSR1, CHEK2, BRCA2, PON1, OGG1, MIC-1, TLR4, and PTEN) or has one or more extra copies of a gene associated with prostate cancer. In some embodiments, the prostate cancer is HER2 positive. In some embodiments, the prostate cancer is HER2 negative.

In some embodiments, the cancer has metastasized and is characterized by circulating tumor cells.

In some embodiments, an SDM of any of Formulas-VI or a carrier-conjugated SDMs comprising a targeting ligand, e.g. an antibody, is employed for the treatment of inflammation or an inflammatory disease. In some embodiments, the inflammation is chronic inflammation. In some embodiments, the inflammation is acute inflammation. In some embodiments, inflammation or inflammatory disease is or is associated with rheumatoid arthritis, osteoarthritis, inflammatory bowel disease, Crohn's disease, ulcerative colitis, sepsis, erythema nodosum leprosum, multiple sclerosis, psoriasis, systemic lupus erythematosis, type I diabetes, atherosclerosis, encephalomyelitis, Alzheimer's disease, stroke, traumatic brain injury, Parkinson's disease or septic shock.

In some embodiments, an SDM of any of Formulas-VI or a carrier-conjugated SDMs comprising a targeting ligand, e.g. an antibody, is employed for the treatment of an autoimmune disease. In some embodiments, the autoimmune disease is Celiac disease, diabetes mellitus type 1, Sarcoidosis, systemic lupus erythematosus (SLE), Sjögren's syndrome. Churg-Strauss Syndrome, Hashimoto's thyroiditis, Graves' disease, idiopathic thrombocytopenic purpura. Addison's Disease, rheumatoid arthritis (RA), Polymyositis (PM), or Dermatomyositis (DM).

### Therapeutic Agents

In some embodiments, a therapeutic agent is selected from: a chemotherapeutic agent, a steroid, an immunotherapeutic agent, a targeted therapy, an anti-inflammatory agent, or a combination thereof.

In some embodiments, a therapeutic agent is a CD79A inhibitor, a CD79B inhibitor, a CD19 inhibitor, a Lyn inhibitor, a Syk inhibitor, a PI3K inhibitor, a Blnk inhibitor, a PLCy inhibitor, a PKCβ inhibitor, or a combination thereof. In some embodiments, a therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacytlase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof. In some embodiments, a therapeutic agent is a B cell receptor pathway inhibitor. In some embodiments, a therapeutic agent is selected from: chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, bendamustine, chlorambucil, chlormethine, cyclophosphamide, ifosfamide, melphalan, prednimustine, trofosfamide, busulfan, mannosulfan, treosulfan, carboquone, thiotepa, triaziquone, carmustine, fotemustine, lomustine, nimustine, ranimustine, semustine, streptozocin, etoglucid, dacarbazine, mitobronitol, pipobroman, temozolomide, methotrexate, permetrexed, pralatrexate, raltitrexed, cladribine, clofarabine, fludarabine, mercaptopurine, nelarabine, tioguanine, azacitidine, capecitabine, carmofur, cytarabine, decitabine, fluorouracil, gemcitabine, tegafur, vinblastine, vincristine, vindesine, vinflunine, vinorelbine, etoposide, teniposide, demecolcine, docetaxel, paclitaxel, paclitaxel poliglumex, trabectedin, dactinomycin, aclarubicin, daunorubicin, doxorubicin, epirubicin, idarubicin, mitoxantrone, pirarubicin, valrubicin, zorubincin, bleomycin, ixabepilone, mitomycin, plicamycin, carboplatin, cisplatin, oxaliplatin, satraplatin, procarbazine, aminolevulinic acid, efaproxiral, methyl aminolevulinate, porfimer sodium, temoporfin, dasatinib, erlotinib, everolimus, gefitinib, imatinib, lapatinib, nilotinib, pazonanib, sorafenib, sunitinib, temsirolimus, alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofuuine, topotecan, tretinoin, vorinostat, diethylstilbenol, ethinylestradiol, fosfestrol, polyestradiol phosphate, gestonorone, medroxyprogesterone, megestrol, buserelin, goserelin, leuprorelin, triptorelin, fulvestrant, tamoxifen, toremifene, bicalutamide, flutamide, nilutamide, aminoglutethimide, anastrozole, exemestane, formestane, letrozole, vorozole, abarelix, degarelix, histamine dihydrochloride, mifamurtide, pidotimod, plerixafor, roquinimex, thymopentin, everolimus, gusperimus, leflunomide, mycophenolic acid, sirolimus, ciclosporin, tacrolimus, azathioprine, lenalidomide, methotrexate, thalidomide, iobenguane, ancestim, filgrastim, lenograstim, molgramostim, pegfilgrastim, sargramostim, interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b, aldesleukin, oprelvekin, BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin, abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus, adalimumab, afelimomab, certolizumab pegol, etanercept, golimumab, infliximab, anakinra, basiliximab, canakinumab, daclizumab, mepolizumab, rilonacept, tocilizumab, ustekinumab, ciclosporin, tacrolimus, azathioprine, lenalidomide, methotrexate, thalidomide, adalimumab, alemtuzumab, bevacizumab, cetuximab, certolizumab pegol, eculizumab, efalizumab, gemtuzumab, ibritumomab tiuxetan, muromonab-CD3, natalizumab, panitumumab, ranibizumab, rituximab, tositumomab, trastuzumab, catumaxomab, edrecolomab, ofatumumab, muromab-CD3, afelimomab, golimumab, ibritumomab tiuxetan, abagovomab, adecatumumab, alemtuzumab, anti-CD30 monoclonal antibody Xmab2513, anti-MET monoclonal antibody MetMab, apolizumab, apomab, arcitumomab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab, a syk inhibitor (e.g., R788), enzastaurin, dasatinib, erlotinib, everolimus, gefitinib, imatinib, lapatinib, nilotinib, pazonanib, sorafenib, sunitinib, temsirolimus, an angiogenesis inhibitor (e.g., GT-11, JI-101, R1530), a kinase inhibitors (e.g., AC220, AC480, ACE-041, AMG 900, AP24534, Arry-614, AT7519, AT9283, AV-951, axitinib, AZD 1152, AZD7762. AZD8055, AZD8931, bafetinib, BAY 73-4506. BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594. KX2-391, linifanib, LY2603618, MGCD265, MK-0457, MK1496, MLN8054, MLN8237, MP470, NMS-1116354, NMS-1286937, ON 01919.Na, OSI-027, OSI-930. Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, S3333333CH 727965, SGI-1176, SGX523, SNS-314, TAK-593, TAK-901, TKI258, TLN-232, TTP607, XL147, XL228, XL281RO5126766, XL418, XL765), an inhibitor of mitogen-activated protein kinase signaling (e.g., U0126, PD98059, PD184352, PD0325901, ARRY-142886, SB239063, SP600125, BAY 43-9006, wortmannin, or LY294002), adriamycin, dactinomycin, bleomycin, vinblastine, cisplatin, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, iimofosine, interleukin I1 (including recombinant interleukin I1, or rIL2), interferon alfa-2a, int interferon alfa-2b, interferon alfa-n 1, interferon alfa-n3, interferon beta-1a, interferon gamma-1b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazoie, nogalamycin, ormaplatin, oxisuran, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. In some embodiments, a therapeutic agent is selected from: 20-epi-1, 25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin 111 derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorlns, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, 9-dioxamycin, diphenyl spiromustine, docosanol, dolasetron, doxifluridine, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-such as for example growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn. O6-benzylguanine, octreotide, okicenone, oligonucleotides, onapristone, ondansetron, ondansetron, oracin, oral cytokine inducer, ormaplatin, osaterone, oxaliplatin, oxaunomycin, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylerie conjugate, raf antagonists, raltitrexed, ramosetron, ras famesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin. SarCNU, sarcophytol A, sargramostim, Sdi 1 mimetics, semustine, senescence derived inhibitor 1, sense oligonucleotides, signal transduction inhibitors, signal transduction modulators, single chain antigen-binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer, mechloroethamine, cyclophosphamide, chlorambucil, busulfan, carmustine, lomusitne, decarbazine, methotrexate, cytarabine, mercaptopurine, thioguanine, pentostatin, mechloroethamine, cyclophosphamide, chlorambucil, meiphalan, ethylenimine, methylmelamine, hexamethlymelamine, thiotepa, busulfan, carmustine, lomusitne, semustine, streptozocin, decarbazine, fluorouracil, floxouridine, cytarabine, mercaptopurine, thioguanine, pentostatin, erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296). ABT-751 (Abbott, also known as E-7010), Altorhyrtins (such as Altorhyrtin A and Altorhyrtin C), Spongistatins (such as Spongistatin 1. Spongistatin 2, Spongistatin 3, Spongistatin 4, Spongistatin 5. Spongistatin 6, Spongistatin 7, Spongistatin 8, and Spongistatin 9), Cemadotin hydrochloride (also known as LU-103793 and NSC-D-669356). Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone). Auristatin PE (also known as NSC-654663). Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate. DZ-3358 (Daiichi), FR-182877 (Fujisawa, also known as WS-9885B), GS-164 (Takeda), GS-198 (Takeda). KAR-2 (Hungarian Academy of Sciences), BSF-223651 (BASF, also known as ILX-651 and LU-223651), SAH-49960 (Lilly/Novartis), SDZ-268970 (Lilly/Novartis). AM-97 (Armad/Kyowa Hakko), AM-132 (Armad), AM-138 (Armad/Kyowa Hakko). IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Ser.HCI, and RPR-258062A), Vitilevuamide, Tubulysin A. Canadensol. Centaureidin (also known as NSC-106969), T-138067 (Tularik, also known as T-67, TL-138067 and TI-138067), COBRA-I (Parker Hughes Institute, also known as DDE-261 and WHI-261), H10 (Kansas State University), H16 (Kansas State University), Oncocidin A1 (also known as BTO-956 and DIME). DDE-313 (Parker Hughes Institute), Fijianolide B. Laulimalide, SPA-2 (Parker Hughes Institute). SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik). Monsatrol, Inanocine (also known as NSC-698666), 3-IAABE (Cytoskeleton/Mt. Sinai School of Medicine), A-204197 (Abbott). T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott). NPI-2350 (Nereus), Taccalonolide A, TUB-245 (Aventis). A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862). A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium. BPR-OY-007 (National Health Research Institutes), and SSR-250411 (Sanofi).

In some embodiments, a therapeutic agent is an anti-inflammatory agent. In some embodiments, a therapeutic agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent, an alkylating agent, an anti-metabolite, a plant alkaloid, a terpenoids, a topoisomerase inhibitor, an antitumour antibiotic, an antibody, a hormonal therapy, an anti-diabetes agent, a leukotriene inhibitor, or combinations thereof. In some embodiments, a therapeutic agent is selected from: alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-BI Antibody (Corixa Corp.), anti-CD66 monoclonal antibody BW 250/183 (NCI. Southampton General Hospital), anti-CD45 monoclonal antibody (NCI, Baylor College of Medicine), antibody anti-anb3 integrin (NCI). BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies). CAT-192 (Human Anti-TGF-Beta1 Monoclonal Antibody, Genzyme), antibody 3F8 (NCI), 177Lu-J591 (Weill Medical College of Cornell University), TB-403 (Biolnvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine. AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1 beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimethyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca). AZD3342 (AstraZeneca), AZD8309 (AstraZeneca)), [(1R)-3-methyl-1-({2S)-3-phenyl-2-[(pyrazin-2-ylcarbonyl)amino]propanoyl}amino)butyl]boronic acid (Bortezomib), AMG-714, (Anti-IL 15 Human Monoclonal Antibody. Amgen), ABT-874 (Anti IL-12 monoclonal antibody, Abbott Labs), MRA(Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol, Pioglitazone, Rosiglitazone, Glimepiride. Glyburide, Chlorpropamide, Glipizide. Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide. Acarbose, Nateglinide, Orlistat, cisplatin; carboplatin: oxaliplatin: mechlorethamine: cyclophosphamide: chlorambucil; vincristine; vinblastine; vinorelbine; vindesine: mercaptopurine; fludarabine: pentostatin; cladribine; 5-fluorouracil (5FU); floxuridine (FUDR); cytosine arabinoside; trimethoprim: pyrimethamine; pemetrexed; paclitaxel; docetaxel: etoposide; teniposide: irinotecan; topotecan; amsacrine; etoposide; etoposide phosphate; teniposide: dactinomycin: doxorubicin; daunorubicin; valrubicine: idarubicine; epirubicin; bleomycin; plicamycin; mitomycin; finasteride; goserelin; aminoglutethimide; anastrozole; letrozole; vorozole: exemestane; 4-androstene-3,6,17-trione (“6-OXO”; 1,4,6-androstatrien-3, 17-dione (ATD): formestane: testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid): CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(41-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy] acetic acid): SA6541 ((R)—S-[[4-(dimethylamino)phenyl]methyl]-N-(3-mercapto-2methyl-1-oxopropyl-L-cycteine); SC-56938 (ethyl-1-[2-[4-(phenylmethyl)phenoxy]ethyl]-4-piperidine-carboxylate): VIA-2291 (Via Pharmaceuticals): WY-47,288 (2-[(1-naphthalenyloxy)methyl]quinoline): zileuton; ZD-2138 (6-((3-fluoro-5-(tetrahydro-4-methoxy-2H-pyran-4yl)phenoxy)methyl)-1-methyl-2(1H)-quinlolinone); doxycycline; or combinations thereof.

In some embodiments, an SDM of any of Formulas-VI or a carrier-conjugated SDMs comprising a targeting ligand, e.g. an antibody, is administered with one or more additional therapeutic agents. In some embodiments, the additional therapeutic agent is selected from among the therapeutic agents listed herein. In some embodiments, the additional therapeutic agent is administered prior to, following, or simultaneously (i.e., concurrently) with an SDM of any of Formulas-VI or a carrier-conjugated SDMs comprising a targeting ligand, e.g. an antibody, provided herein.

Imaging Uses

The SDMs of Formulas I-VI and carrier-conjugated SDMs comprising a targeting ligand, e.g. an antibody, allow the targeted delivery of imaging agents to specific cells and/or tissues (e.g., cancerous tissues). The SDMs comprise a basic peptide sequence (B) which is designed to be transported across a cellular membrane or retained by tissue, an acidic peptide sequence (A) which inhibits uptake and retention of peptide B into cells, a linker X which is cleavable under specific conditions, imaging moieties bound to peptides A and B, or X and a macromolecular carrier. In some embodiments, cleavage of the linker X linker frees peptide B from peptide A and allows the transport of peptide B (and any imaging moieties attached thereto) across a cellular membrane or retention of B to tissue. In some embodiments, the SDMs enable targeted delivery of one or more imaging agents to a cell or tissue. In some embodiments, targeted delivery of an imaging agent to a cell or tissue enables a medical professional to visualize/image a specific tissue.

In some embodiments, targeted delivery of an imaging agent to a cell or tissue enables a medical professional to visualize/image a specific tissue (e.g., cancerous tissue). In some embodiments, targeted delivery of an imaging agent to a cell or tissue enables a medical professional to remove (or, surgically excise) the tissue of interest (e.g., cancerous tissue). In some embodiments, targeted delivery of an imaging agent to a cell or tissue enables a medical professional to remove (or, surgically excise) the tissue of interest (e.g., cancerous tissue) with a decrease in surgical margins. In some embodiments, targeted delivery of an imaging agent to a cell or tissue enables a medical professional to remove (or, surgically excise) a tumor/cancerous tissue and decreases the chance that some of the tumor/cancerous tissue will not be removed. In some embodiments, targeted delivery of an imaging agent to a cell or tissue enables a medical professional to maximally debulk a tumor/cancerous tissue. In some embodiments, targeted delivery of an imaging agent to cancerous breast tissue decreases the chances of an unnecessary operations and re-operations.

In some embodiments, targeted delivery of an imaging agent to a cell or tissue enables a medical professional to more accurately sample (e.g., biopsy (e.g., excision biopsy, incision, biopsy, aspiration biopsy, or needle biopsy)) tissue of interest (e.g., cancerous tissue). In some embodiments, targeted delivery of an imaging agent to a cell or tissue enables a medical professional to visualize/image a specific tissue (e.g., cancerous tissue) within an excised tissue containing healthy tissue. Enabling identification of target tissue (e.g., cancerous tissue) can guide the pathologist on where to section of pathological evaluation and decreases the chances of a pathologist missing unhealthy tissue (e.g., cancerous tissue) and sampling healthy tissue which may produce a false negative. In some embodiments, tissue (e.g., cancerous tissue) removed following use of a compound of Formula I is used to prepare a pathology section or slide. In some embodiments, cancerous tissue removed following use of a compound of Formula I is used to prepare a pathology section or slide which is used to diagnose a tissue as malignant or benign.

In some embodiments, targeted delivery of an imaging agent to cancerous breast tissue enables a medical professional to accurately stage cancer enabling medical treatment decisions. In some embodiments, targeted delivery of an imaging agent to cancerous tissue enables a medical professional to observe the size of a tumor (cancerous tissue) or the spread (e.g., metastatic lesions) of cancerous tissue. In some embodiments, targeted delivery of an imaging agent to a cell or tissue enables a medical professional to design an efficacious treatment regimen.

In some embodiments, a selective delivery molecule according to Formula I comprising an imaging agent is employed in guided surgery. In some embodiments, the selective delivery molecule preferentially localized to cancerous, or other pathological tissues with up-regulated protease activity (e.g. tissues undergoing inflammatory response). In some embodiments, a selective delivery molecule according to Formula I comprising an imaging agent is employed in a guided surgery to remove colorectal cancer. In some embodiments, guided surgery employing the selective delivery molecule allows a surgeon to excise as little healthy (i.e., non-cancerous) tissue as possible. In some embodiments, guided surgery employing the selective delivery molecule allows a surgeon to visualize and excise more cancerous tissue than the surgeon would have been able to excise without the presence of the selective delivery molecule. In some embodiments, the surgery is fluorescence-guided surgery.

### Imaging Agents

In some embodiments, an imaging agent is a dye. In some embodiments, an imaging agent is a fluorescent moiety. In some embodiments, a fluorescent moiety is selected from: a fluorescent protein, a fluorescent peptide, a fluorescent dye, a fluorescent material or a combination thereof.

All fluorescent moieties are encompassed within the term “fluorescent moiety.” Specific of fluorescent moieties given herein are illustrative and are not meant to limit the fluorescent moieties for use with the targeting molecules disclosed herein.

of fluorescent dyes include, but are not limited to, xanthenes (e.g., rhodamines, rhodols and fluoresceins, and their derivatives); bimanes: coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane: xanthene: pyrylium: carbostyl; perylene; acridone: quinacridone: rubrene; anthracene; coronene: phenanthrecene: pyrene: butadiene: stilbene: porphyrin; pthalocyanine; lanthanide metal chelate complexes; rare-earth metal chelate complexes; and derivatives of such dyes.

of fluorescein dyes include, but are not limited to, 5-carboxyfluorescein, fluorescein-5-isothiocyanate, fluorescein-6-isothiocyanate and 6-carboxyfluorescein.

of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®).

of cyanine dyes include, but are not limited to, Cy3, Cy3B, Cy3.5, Cy5, Cy5.5, Cy7, IRDYE680, Alexa Fluor 750, IRDye800CW, ICG.

of fluorescent peptides include GFP (Green Fluorescent Protein) or derivatives of GFP (e.g., EBFP, EBFP2, Azurite, mKalamal, ECFP, Cerulean, CyPet, YFP, Citrine. Venus, YPet).

Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc.

In some embodiments, the imaging agent is labeled with a positron-emitting isotope (e.g., ¹⁸F) for positron emission tomography (PET), gamma-ray isotope (e.g., ^{99m}Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the imaging agent is labeled with: a gadolinium chelate, an iron oxide particle, a super paramagnetic iron oxide particle, an ultra small paramagnetic particle, a manganese chelate or gallium containing agent.

of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the imaging agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indium chelate.

of Tc chelates include, but are not limited to HYNIC, DTPA, and DOTA.

In some embodiments, the imaging agent contains a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu, and others.

### Starting Materials

Disclosed herein, in certain embodiments, are molecules of Formula VII, having the structure:

A₁-X₁-B₁; Formula VII

wherein,

X₁ is a cleavable linker;

A₁ is a peptide with a sequence comprising 5 to 9 acidic amino acids and having a first reactive amino acid moiety c_{A}

B₁ is a peptide with a sequence comprising 7 to 9 basic amino acids and having a second reactive amino acid moiety c_{B}; and

A₁-X₁-B₁ has a third reactive amino acid moiety c_{M} of A₁ or X₁; and wherein c_{A} is capable of reacting with a first cargo moiety comprising D_{A}, c_{B} is capable of reacting with a second cargo moiety comprising D_{B}, and c_{M} is capable of reacting with a macromolecular carrier comprising M to form a molecule of Formula I. In some embodiments, the c_{A}, c_{B}, and c_{M} have functional groups that are orthogonally reactive. In some embodiments, c_{A}, c_{B}, and c_{M} are each independently selected from a naturally-occurring amino acid or a non-naturally-occurring amino acid. In some embodiments, c_{A}, c_{B}, and c_{M} are each independently selected from a D amino acid, a L amino acid, an α-amino acid, a β-amino acid, or β-amino acid. In some embodiments, c_{A}, c_{B}, and c_{M} are each independently selected from any amino acid having a free thiol group, any amino acid having a N-terminal amine group, and any amino acid with a side chain capable of forming an oxime or hydrazone bond upon reaction with a hydroxylamine or hydrazine group. In some embodiments, c_{A}, c_{B}, and c_{M} are each independently selected from D-cysteine, D-glutamate, lysine, and para-4-acetyl L-phenylalanine. In some embodiments, c_{B} is any amino acid having a free thiol group. In some embodiments, c_{B} is D-cysteine. In some embodiments, c_{A} is any amino acid having a N-terminal amine group. In some embodiments, c_{A} is D-glutamate. In some embodiments, c_{A} is lysine. In some embodiments, c_{M} is any amino acid with a side chain capable of forming an oxime or hydrazone bond upon reaction with a hydroxylamine or hydrazine group. In some embodiments, c_{M} is para-4-acetyl L-phenylalanine.

As used herein, “orthogonally reactive” means a plurality of groups can be attached to a molecule via a sequence of reactions that do not cross react enabling specific attachment of each group in the presence of the others. In some embodiments, the three groups (D_{A}, D_{B}, and D_{M}) are able to be attached to A₁-X₁-B₁ via c_{A}, c_{B}, and c_{M} using a sequence of 3 independent reactions that do not cross react so that each group is attached to only one site of A₁-X₁-B₁.

Disclosed herein, in certain embodiments, is a molecule having the amino acid sequence:

(D-Glu)₅-F(4-Ac)-o-Pro-Leu-Gly-Cys(_{Me})-Ala-Gly-(D-Arg)₈-(D-Cys)

wherein o represent 5-(amino-3-oxapentanoyl); F_{(4-Ac)} represent para-acetyl-(L)-phenylalanine; and C_{(Me)} represents S-methyl-(L)-cysteine.

In some embodiments, the molecule further comprises a polyethylene glycol (PEG) polymer. In some embodiments, the PEG polymer is covalently linked to the molecule at the F(4-Ac) subunit. In some embodiments, the molecule comprises groups that can be orthogonally reacted. In some embodiments, the groups that can be orthogonally reacted are chosen from: an amine, thiol and an acetyl phenylalanine. In some embodiments, the molecule comprises an amine, a thiol, and an acetyl phenylalanine.

In some embodiments, the PEG polymer has an average molecular weight of 500 daltons. In some embodiments, the PEG polymer has an average molecular weight of 2,000 daltons. In some embodiments, the PEG polymer has an average molecular weight of 3,000 daltons. In some embodiments, the PEG polymer has an average molecular weight of 4,000 daltons. In some embodiments, the PEG polymer has an average molecular weight of 5.000 daltons. In some embodiments, the PEG polymer has an average molecular weight of 10,000 daltons. In some embodiments, the PEG polymer has an average molecular weight of 12,000 daltons. In some embodiments, the PEG polymer has an average molecular weight of 15,000 daltons. In some embodiments, the PEG polymer has an average molecular weight of 20,000 daltons. In some embodiments, the PEG polymer has an average molecular weight of 30,000 daltons. In some embodiments, the PEG polymer has an average molecular weight of 40.000 daltons.

Disclosed herein, in certain embodiments, is the use of the molecule in the synthesis of a molecule according to Formulas I-VI.

Disclosed herein, in certain embodiments, is a molecule having the amino acid sequence:
(D-Glu)₅-o-Pro-Leu-Glys-Cys₍ₘₑ₎-Ala-Gly-(D-Arg)₈-(D-Cys)-[PEG_{(3K)}]
   wherein all glutamates and arginines are D-amino acids; o represents 5-(amino-3-oxapentanoyl); C(me) represents S-methyl-(L)-cysteine; and PEG_{(3K)} represents α-amino-o-amide poly(ethylene glycol) with an average three thousand Dalton molecular weight. In some embodiments, the molecule further comprises a fluorescent moiety. Disclosed herein, in certain embodiments, is the use of the molecule in the synthesis of a molecule according to Formulas I-VI.

### EXAMPLES

These are provided for illustrative purposes only and not to limit the scope of the claims provided herein.

### Materials and Methods

All reaction solvents were freshly opened Aldrich “Sure-Seal” quality. All the reagents were reagent-grade and used without further purification unless otherwise indicated. HPLC-grade acetonitrile was purchased from Fisher Scientific (Phillipsburg, Pa.). Water used in HPLC was collected through Milli-Q water purification system (Millipore, Bedford, Mass.). PBS-EDTA buffer was purchased from Teknova (Hollister, Calif.). α-Mercaptoethyl-ω-methoxy, poly-oxyethylene (average molecular weight around 2,000, 5,000, 20,000 and 40,000 daltons) [mPEG(2K)-SH, mPEG(5K)-SH, mPEG(20K)-SH, mPEG(40K)-SH] and α-aminoxyl-ω-methoxy, polyoxyethylene (average molecular weight around 2,000, 5,000, 20,000 and 40,000) [mPEG(2K)-ONH₂, mPEG(5K)-ONH₂, mPEG(20K)-ONH₂, mPEG(40K)-ONH₂] were purchased from NOF America Corporation (Irvine, Calif.). Compound 1 was supplied by GL Biochem Ltd. (Shanghai, China). Doxorubicin was purchased from NuBlocks LLC (Oceanside, Calif.). Lyophilized peptide P1-P18 was supplied by PolyPeptide Group (San Diego, Calif.). 3-Maleimidopropionic acid pentafluorophenyl ester 7 was purchased from Molecular Biosciences (Boulder, Colo.). Compound 17 was purchased from MedChem Express (Princeton, Colo.).

LC-MS analysis was carried out on an Agilent 1200 SL series in combination with AB SCIEX API 3200, equipped with CTC PAL autosampler operating at 4° C., a vacuum degasser, binary pump, UV-VIS detector, associated Analyst 1.5 analytical software and a Phenomenex column (Kinetex 2.6p C18 100A, 100×2.1 mm) or a Waters 2695 separation module equipped with a Waters 2487 dual λ absorbance detector in combination with Finnigan LCQ Deca XP mass spectrometer. The equipment is associated with Xcalibur analytical software and Peeke Scientific columns (Titan 200 5 μm, C18-MC, 50/100×2.1 mm).

Preparation HPLC were carried out on an Agilent system (Agilent 1200 series) and a Thermo Scientific column (Hypersil Gold C18, 5μ, 250×10 mm), or a Waters Delta Prep preparative HPLC System and a Varian column (F75L, C18, 15μ, 1200 g), or a Waters PrepLC System equipped with a Waters 2487 dual X absorbance detector, Fraction Collector 111, Masslynx software and a Thermo Scientific column (Hypersil Gold C18, 5μ, 250×10 mm) or a Phenomenex column (luna, C18(2), 5μ, 100A AX 150×30 mm). The mobile phase consisted of a water (0.05% TFA)solvent A)/acetonitrile (0.05% TFA)(solvent B) gradient unless otherwise specified. Centrifugation was carried out at 4° C. on an Eppendorf centrifuge 5417R or a Beckman Microfuge® 18. Lyophilization was carried out on a Labconco FreeZone 4.5.

### Example 1: Synthesis of Intermediate 5

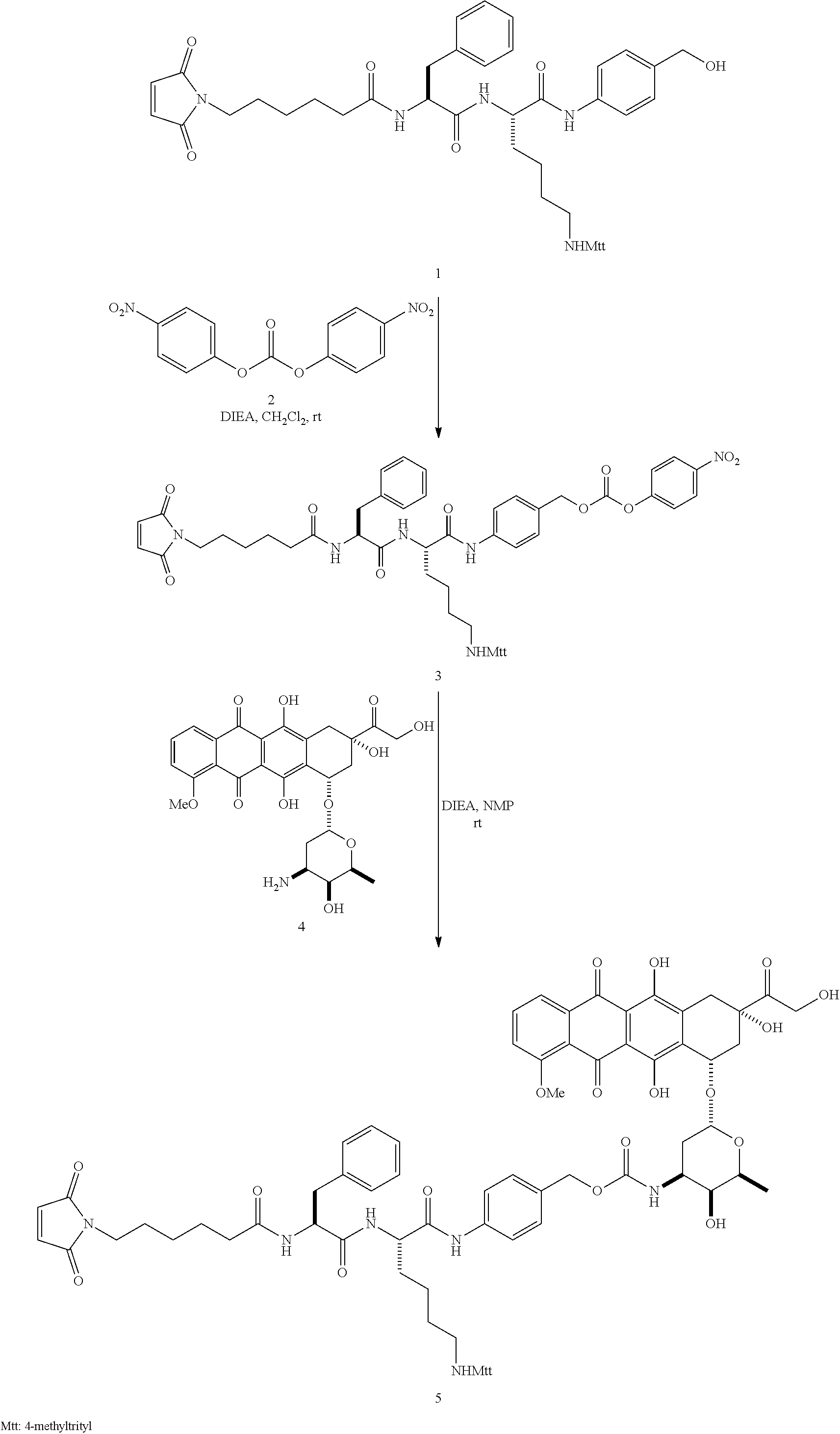

### Synthesis of Intermediate 3

To a solution of peptide 1 (1.01 g, 1.2 mmol) and 2 (2.0 g, 6.6 mmol) in CH₂Cl₂ (40 mL) at room temperature was added DIEA (0.65 m, 3.7 mmol). The mixture was stirred at room temperature for 1 day. After the solvent was removed, the residue was dissolved in EtOAc (100 mL). The organic layer was washed with sodium acetate buffer (pH 5, 100 mL, 1 M), water (100 mL) and brine (50 mL), dried, and evaporated. The residue was purified by flash chromatography on silica gel, eluting with 1:1 ethyl acetate/hexane, to afford 3 (920 mg, 76%) as a light yellow oil. ¹H NMR (500 MHz, CDCl₃): δ 8.46 (br. s, 1H), 8.25 (d, J=9.0 Hz, 2H), 8.10 (d, J=9.0 Hz, 1H), 7.60 (d, J=8.0 Hz, 2H), 7.43-7.34 (m, 7H), 7.30 (d, J=8.0 Hz, 2H), 7.23 (t, J=8.0 Hz, 4H), 7.17-7.11 (m, 7H), 7.04 (d, J=8.0 Hz, 2H), 6.82 (d, J=9.0 Hz, 2H), 6.60 (s, 2H), 5.25 (s, 2H), 4.65 (q, J=7.0 Hz, 1H), 4.43 (q, J=7.0 Hz, 1H), 3.43 (1, J=7.0 Hz, 2H), 3.09 (dd, J=14.0; 7.0 Hz, 1H), 3.03 (dd, J=14.0; 7.0 Hz, 1H), 2.28 (s, 3H), 2.13 (t, J=7.5 Hz, 2H), 2.08 (t, J=7.0 Hz, 2H), 1.89-1.92 (m, 1H), 1.46-1.65 (m, 6H), 1.27-1.32 (m, 4H), 1.18-1.27 (m, 2H): ¹³C NMR (125 MHz, CDCl₃): δ 173.9, 171.7, 171.0, 169.6, 163.8, 155.8, 152.7, 146.6, 145.6, 143.4, 138.7, 136.0, 135.9, 134.3, 130.4, 129.9, 129.3, 128.8, 128.7, 128.7, 127.9, 127.6, 126.5, 126.4, 125.5, 122.0, 120.4, 120.3, 116.2, 70.9, 70.8, 55.1, 54.5, 43.5, 38.1, 37.7, 36.3, 31.5, 30.7, 28.3, 26.3, 25.0, 23.7, 21.1, 19.3; MS (ESI): m/e 1013 [M+H]1, 1036 [M+Na]+.

### Synthesis of Intermediate 5

To a solution of intermediate 3 (137 mg, 0.14 mmol) and doxorubicin 4 (80 mg, 0.14 mmol) in N-methyl-2-pyrrolidone (NMP) (5 mL) at room temperature was added DIEA (25 μL, 0.14 mmol). The mixture was stirred at room temperature in the dark for 72 h. To the reaction mixture was added EtOAc (100 mL). The organic layer was washed with water (100 mL×2) and brine (50 mL), dried, and evaporated. The residue was purified by flash chromatography on silica gel, eluting with 3:1 ethyl acetate/hexane, to afford 5 (118 mg, 76%) as an orange powder. ¹H NMR (500 MHz, DMSO-d₆): δ 14.0 (s, 1H), 13.3 (s, 1H), 8.13 (d, J=8.0 Hz, 1H), 7.97 (d, J=8.0 Hz, 1H), 7.93-7.89 (m, 2H), 7.66-7.64 (m, 1H), 7.53 (d, J=8.0 Hz, 2H), 7.47-7.43 (m, 6H), 7.30-7.15 (m, 16H), 6.99 (s, 2H), 6.81 (d, J=8.0 Hz, 1H), 5.45 (br. s, 1H), 5.22 (d, J=3.0 Hz, 1H), 4.95 (t, J=4.0 Hz, 1H), 4.90 (d, J=12.5 Hz, 1H), 4.88 (d, J=12.5 Hz, 1H), 4.57 (s, 2H), 4.52-4.46 (m, 1H), 4.32-3.30 (m, 1H), 4.16-4.14 (m, 1H), 3.98 (s, 4H), 3.44 (s, 1H), 2.39 (t, J=7.0 Hz, 3H), 2.99-2.94 (m, 3H), 2.73-2.70 (m, 3H), 2.31 (s, 3H), 2.22-2.13 (m, 2H), 2.00-1.93 (m, 2H), 1.87-1.82 (m, 1H), 1.75-1.45 (m, 5H), 1.42-1.28 (m, 4H), 1.25-1.15 (m, 3H), 1.12 (d, J=6.5 Hz, 3H), 1.10-0.98 (m, 2H); ¹³C NMR (125 MHz, DMSO-d₆): δ 213.5, 186.6, 186.5, 172.1, 171.4, 170.9, 170.1, 160.8, 156.0, 155.2, 154.5, 138.2, 137.8, 136.2, 135.5, 134.7, 134.4, 134.1, 132.0, 129.1, 129.0, 128.6, 128.5, 128.4, 127.9, 126.1, 120.1, 119.7, 119.1, 119.0, 110.8, 110.7, 100.1, 75.0, 69.8, 68.0, 66.6, 64.8, 63.6, 56.6, 53.8, 53.2, 47.1, 37.2, 36.9, 36.7, 34.9, 32.1, 31.4, 29.8, 27.6, 25.7, 25.5, 24.6, 22.6, 20.4, 16.9; MS (ESI): m/e 1417 [M+H]⁺, 1440 [M+Na]⁺.

### Example 2: Synthesis of SDM-101 and SDM-145

### Synthesis of Intermediate 6

To a solution of intermediate 5 (3.8 mg, 2.7 μmol) and peptide P-2 (8.0 mg, 2.2 μmol) in DMF (0.7 mL) at room temperature in the dark was added N-methylmorpholine (NMM) (5 μL, 46 μmol) with stirring. The reaction was followed by LC-MS and completed in 1 h. The mixture was directly used in the next step without further purification. MS (ESI): m/e 1349 [M+3H]³.

### Synthesis of Intermediate 8

To the reaction mixture above was added 3-maleimidopropionic acid-Pfp ester 7 (1 mg, 3.0 μmol). The resulting mixture was stirred at room temperature in the dark for 3 h. Purification by RP-HPLC afforded intermediate 8 (7.7 mg, 83% for two steps). MS (ESI): me 1400 [M+3H]³⁺.

### Synthesis of SDM-101

A stirred solution of intermediate 8 (2.4 mg, 0.57 μmol) and thioanisole (10 μL, 85 μmol) in CH₂Cl₂ (1 mL) was treated with trifluoroacetic acid (5 μL, 65 μmol). The mixture was stirred at room temperature in the dark for 30 min. After the solvent was removed, the residue was purified by RP-HPLC to afford SDM-101 (0.6 mg, 27%) as a free flowing red powder after lyophilization. MS (ESI): m/e 1315 [M+3H]³.

### Synthesis of SDM-145

The mixture of SDM-101 (0.6 mg, 0.15 μmol) and mPEG(40K)-SH 9 (5 mg, 0.12 μmol) in PBS-EDTA buffer (0.5 mL, 137 mM NaCl, 7 mM Na₂HPO₄, 3 mM KCl, 1.4 mM K₃PO₄, 4 mM EDTA, pH 7.4) was stirred at room temperature in the dark for 5 h. Purification by RP-HPLC afforded SDM-145 as a red powder after lyophilization (3.0 mg, 60%).

### Example 3: Cleavage of SDM-145 by hMMP-9

Conjugate SDM-145 (3.0 mg) was dissolved in water (135 μL) to make a stock solution (0.5 mM). To a TCNB buffer (50 mM tris, 10 mM CaCl₂, 150 mM NaCl, 0.05% Brij35, pH 7.5, 480 μL) in a HPLC sample vial was added SDM-145 stock solution (10 μL) and hMMP-9 (10 μL, 100 nM) purchased from EMD Millipore (Billerica, Mass.). The resulting solution was gently mixed well and incubated 37° C.

Aliquots (15 uL) were removed at various time points and injected into an LC-MS equipped with a fluorescence spectrometer (ex: 480 nm; em: 560 nm). The cleavage reaction was complete after 17 hour. The peak at retention time ˜9.4 min was confirmed to be the cleaved poly-arginine fragment by MS (ESI): m/e 1388.9 [M+2H]²⁺.

### Example 4: Cleavage of SDM-145 by Cathepsin B

Conjugate SDM-145 (3.0 mg) was dissolved in water (135 μL) to make a stock solution (0.5 mM). To a sodium acetate buffer (25 mM NaAc, 1 mM EDTA, pH 5.0, 480 μL) in a HPLC sample vial was added conjugate SDM-145 stock solution (10 μL) and Cathepsin B, human liver (10 μL, 100 nM) purchased from EMD Millipore (Billerica, Mass.). The resulting solution was gently mixed well and incubated 37° C.

Aliquots (15 uL) were removed at various time points and injected into an LC-MS equipped with a fluorescence spectrometer (ex: 480 nm; em: 560 nm). The cleavage reaction was complete after 17 hour. The peak at retention time ˜9.0 min was confirmed to be the freed doxorubicin by MS (ESI): m/e 566.4 [M+Na]⁺.

### Example 5: Synthesis of SDM-143 and SDM-146

### Synthesis of Intermediate 10

To a solution of intermediate 5 (230 mg, 0.16 mmol) and peptide P-3 (700 mg, 0.17 mmol) in DMF (5.0 mL) at room temperature in the dark was added N-methylmorpholine (NMM) (200 μL, 1.8 mmol) with stirring. The reaction was followed by LC-MS and completed in 1 h. Purification by RP-HPLC afforded intermediate 10 (426 mg, 62%) MS (ESI): m/e 1412.6 [M+3H]³⁺.

### Synthesis of Intermediate 11

To the solution of 10 (148 mg, 28.7 μmol) in anhydrous DMF (4 mL) was added 3-maleimidopropionic acid-Pfp ester 7 (20 mg, 59.7 μmol) and NMM (100 uL, 0.9 mmol). The resulting mixture was stirred at room temperature in the dark for 20 h. Purification by RP-HPLC afforded intermediate 11 (148 mg, 97%). MS (ESI): me 1463 [M+3H]³⁺.

### Synthesis of SDM-143

A stirred solution of intermediate 11 (120 mg, 23.3 μmol) in CH₂Cl₂ (40 mL) was treated with trifluoroacetic acid (100 μL). The mixture was stirred at room temperature in the dark for 5 h. After the solvent was removed, the residue was purified by RP-HPLC to afford SDM-143 (102 mg, 87%) as a free flowing red powder after lyophilization. MS (ESI): m/e 1377.9 [M+3H]³⁺, 1529.6 [M+4TFA+3H]³⁺.

### Synthesis of SDM-146

The mixture of SDM-143 (26 mg, 5.0 μmol) and mPEG(40K)-SH 9 (225 mg, 5.6 μmol) in PBS buffer (5.0 mL, pH 7.4) was stirred at room temperature in the dark for 5 h. Purification by RP-HPLC afforded SDM-146 as a red powder after lyophilization (172 mg, 77%).

### Example 6: Synthesis of SDM-147

### Synthesis of SDM-147

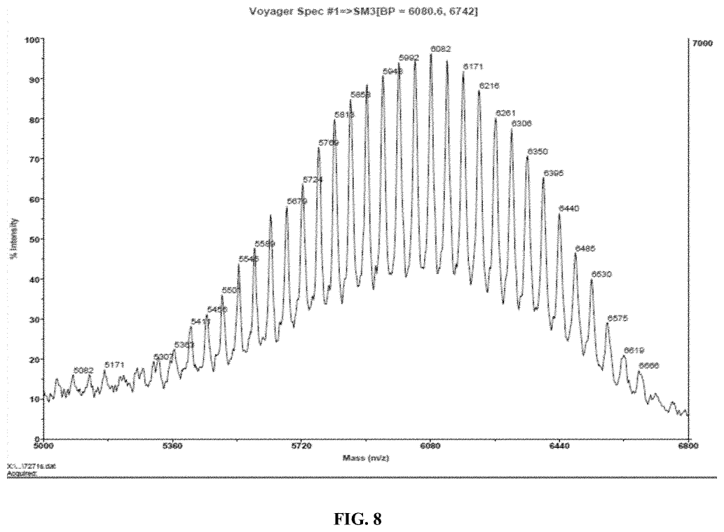

The mixture of SDM-143 (117 mg, 22.7 μmol) and mPEG(2K)-SH 12 (63 mg, 29.4 μmol) in PBS buffer (5.0 mL, pH 7.4) was stirred at room temperature in the dark for 5 h. Purification by RP-HPLC afforded SDM-147 as a red powder after lyophilization (172 mg, 77%). MALDI-TOF: ion clusters observed between approximately m/z 5300-6700, with 44 Da differences and centered at approximately m/z 6082 (FIG. 8).

### Example 7: Synthesis of SDM-144 and SDM-148

### Synthesis of Intermediate 14

To a solution of intermediate 5 (115 mg, 0.08 mmol) and peptide P-16 (200 mg, 0.04 mmol) in DMF (5.0 mL) at room temperature in the dark was added N-methylmorpholine (NMM) (70 μL, 0.63 mmol) with stirring. The reaction was followed by LC-MS and completed in 1 h.

After the addition of 3-maleimidopropionic acid-Pfp ester 7 (30 mg, 89.6 μmol), the resulting mixture was stirred at room temperature in the dark for 18 h. Purification by RP-HPLC afforded intermediate 14 (110 mg, 45% for 2 steps) MS (ESI): m/e 1618.8 [M+3H]³⁺.

### Synthesis of SDM-144

A stirred solution of intermediate 14 (108 mg, 17.7 μmol) in CH₂Cl₂ (40 mL) was treated with trifluoroacetic acid (100 μL). The mixture was stirred at room temperature in the dark for 3 h. After the solvent was removed, the residue was purified by RP-HPLC to afford SDM-144 (84 mg, 81%) as a free flowing red powder after lyophilization. MS (ESI): m/e 1150.3 [M+4H]⁴⁺, 1533.2 [M+3H]⁺, 1684.5 [M+4TFA+3H]³⁺.

### Synthesis of SDM-148

The mixture of SDM-144 (28 mg, 4.8 μmol) and mPEG(2K)-SH 12 (9.0 mg, 4.2 μmol) in PBS buffer (5.0 mL, pH 7.4) was stirred at room temperature in the dark for 5 h. Purification by RP-HPLC afforded SDM-148 as a red powder after lyophilization (33 mg, 86%).

### Example 8: Synthesis of SDM-149

### Synthesis of Intermediate 16

To a solution of Peptide P-3 (60 mg, 0.15 mmol) in glycine buffer (0.1 M, 20 mM aniline, pH 3.0, 2.0 mL) at room temperature was added mPEG(2K)-ONH₂ 15 (30 mg, 0.14 mmol) with stirring. The reaction was followed by LC-MS and completed in 15 h. Purification by RP-HPLC afforded intermediate 16 (70 mg, 83%).

### Synthesis of SDM-149

To a mixture of 17 (8.7 mg, 10 μmol) and 16 (42 mg, 6.9 μmol) in anhydrous DMF (1 mL) was added N-methylmorpholine (10 μL). The mixture was stirred at room temperature for 20 h. Purification by RP-HPLC (mobile phase A: water; mobile phase B: acetonitrile) afforded SDM-149 (22 mg, 46%).

### Example 9: Synthesis of SDM-150

### Synthesis of SDM-ISO

The mixture of 18 (8.7 mg, 6.8 μmol) and 16 (42 mg, 6.9 μmol) in PBS (2.0 mL, pH 7.4)-acetonitrile (1 mL) mixed solvent was stirred at room temperature for 2 h. Purification by RP-HPLC afforded SDM-150 (40.2 mg, 81%).

### Example 10: Synthesis of Cathespin B Labile ACPP-Cortisone Conjugate

A Cathespin B Labile ACPP-Cortisone conjugate was synthesized as follows:

### Example 11: Breast Cancer Mouse Therapeutic Model and Assay

Female BALB/c mice (8-10 weeks old) purchased from Harlan (Indianapolis, Ind., 46259) or Charles River (Wilmington, Mass., 01887) were used after 4-7 day of acclimatization period. All studies were conducted at research facility under the Institutional Animal Care and Use Committee (IACUC) approved protocol # EBI 1-002-009. On the first day of study, animals were weighed and assessed for health status. Only animals with no sign of disease were selected for the study. Each involved animal was lightly anesthetized with a mixture of ketamine/xylazine administered intraperitoneally to subdue voluntary movement. Highly metastatic 4T1 tumor cells (ATCC® Number CRL-2539™) suspended in DPBS/Matrigel™ (1:1 vol) were then injected subcutaneously (4×10⁵ tumor cells/50 μL/mouse) into the right upper mammary fat pad of the lightly anesthetized animal. Each involved animal was then allowed to recover from anesthesia, housed back in the vivarium and kept under controlled environmental conditions.

Ten days after the subcutaneous implantation of 4TI tumor cells into the upper mammary fat pad, the tumor size (width and length) of each involved animal was measured using a Mitutoyo 500-196-20 Absolute Digimatic Digital Caliper (Mitutoyo Corporation, Kanagawa, 213-0012. Japan) and the individual tumor volume (mm³) calculated as follows: tumor volume=width²×length/2. Tumor-bearing mice were then divided into 3-5 experimental groups (n=3-4 tumor-bearing mice/group) based on identical averaged (Mean±SEM) tumor volume before the intravenous administration of vehicle or test compound. For each experimental group, each involved tumor-bearing mouse was restrained using the tail rotating tail injector (Cat.# RTI, Braintree Scientific, Inc., Braintree, Mass. 02185) and dosed with vehicle or test compound administered intravenously using a 28 G^{1/2} insulin syringe (Cat.#14-826-79, Becton Dickinson and Company, Franklin Lakes, N.J. 07417). Vehicle or test compound was administered intravenously every other day or every three days based on the pharmacokinetic profile of the screened chemical entity. Individual body weight and tumor volume were systematically recorded on the day of dosing.

Twelve days after the first dosing, the individual body weight was recorded and the tumor size measured and the volume calculated as above. Each involved animal was then terminally anesthetized with the mixture of ketamine/xylazine. A blood sample was collected from each anesthetized tumor-bearing by cardiac puncture using a 1 cc Terumo Syringe Tuberculin with needle (25 G×⅝″, Ref: SS-01T2516, Somerset, N.J. 08873) and processed for plasma separation. The tumor was then dissected and collected. Plasma and tumor samples were kept frozen (−80° C.) before being used for in vitro analyses.

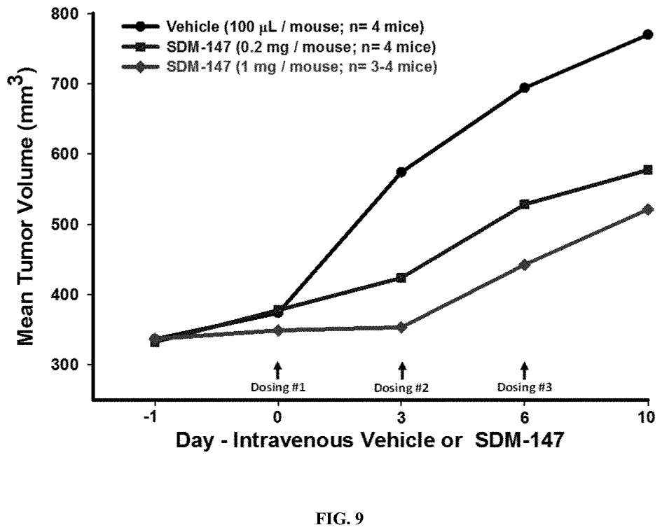

Averaged tumor volume from individual experimental groups were compared to determine the therapeutic effect of the test compound. As shown in FIG. 9, SDM-147 reduces the tumor volume in the murine 4T1 breast cancer model compared to vehicle.

The examples and embodiments described herein are for illustrative purposes only and various modifications or changes suggested to persons skilled in the art are to be included within the spirit and purview of this application and scope of the appended claims.

1.-17. (canceled)

18. A selective delivery molecule conjugate comprising:
(a) a selective delivery molecule of Formula V, having the structure:
A-[c_{M}-M]-X-B-Y-[c_{B}-D_{B}] Formula V
wherein,
X is a linker cleavable by an extracellular protease;
Y is a linker cleavable by an intracellular protease or is a self-immolative spacer;
A is a peptide with a sequence comprising 5 to 9 acidic amino acids;
B is a peptide with a sequence comprising 7 to 9 basic amino acids;
c_{B} and c_{M} each independently comprise 0-1 amino acid;
M is 0-1 macromolecule selected from a protein, a natural polymer, a synthetic polymer, a dendrimer, dextran, a polyethylene glycol (PEG) polymer, albumin, or a combination thereof;
D_{B} is a therapeutic agent or an imaging agent,
wherein [c_{M}-M] is bound to at any position on A or X, and Y-[c_{B}-D_{B}] is bound to any amino acid on B.

19. The molecule of claim 18, further comprising a carrier or targeting ligand, wherein the carrier or targeting ligand is covalently bound to the selective delivery molecule.

20. (canceled)

21. (canceled)

22. The molecule of claim 19, wherein the targeting ligand binds to a tumor antigen or tumor-specific receptor.

23. The molecule of claim 19, wherein the targeting ligand is gemtuzumab, inotuumab, trastuzumab, lorvotuzumab, imgn388, SAR3419, Bi1B062, brentixumab, glembatumumab, SGN-75, PSMA ADC, ASG-5ME or mdx-1203.

24. The molecule of claim 18, wherein the therapeutic agent is a chemotherapeutic agent, a cytotoxin, a steroid, an immunotherapeutic agent, a targeted therapy, or an anti-inflammatory agent.

25. The molecule of claim 18, wherein the therapeutic agent is doxorubicin, calicheamicin, maytansinoid, auritstatin or cortisone.

26. (canceled)

27. (canceled)

28. The molecule of claim 18, wherein c_{B} and c_{M} are each independently selected from D-cysteine, D-glutamate, lysine, and para-4-acetyl L-phenylalanine.

29. (canceled)

30. (canceled)

31. The molecule of claim 18, wherein X comprises an amino acid sequence selected from: PLGLAG (SEQ ID NO: 2), PLG-C(me)-AG (SEQ ID NO: 1), RPLALWRS (SEQ ID NO: 7), ESPAYYTA (SEQ ID NO: 8), DPRSFL (SEQ ID NO: 9), PPRSFL (SEQ ID NO: 10), RLQLKL (SEQ ID NO: 11), and RLQLK(Ac) (SEQ ID NO: 12).

32. (canceled)

33. The molecule of claim 18, wherein Y is cleavable by a lysosomal protease.

34. The molecule of claim 18, wherein Y is cleavable by a cathepsin or a caspase.

35. The molecule of claim 18, wherein Y is cleavable by Cathepsin B.

36. (canceled)

37. The molecule of claim 18, wherein Y comprises a PABC spacer, a PABOH spacer, a BHMS spacer or any derivative thereof.

38. (canceled)

39. (canceled)

40. The molecule of claim 18, wherein M is selected from PEG 1 kDa, PEG 2 kDa, PEG 3 kDa, PEG 4 kDa, PEG 5 kDa, PEG 10 kDa, PEG 12 kDa, PEG 15 kDa, PEG 20 kDa, PEG 30 kDa, and PEG40 kDa.

41. The molecule of claim 18, wherein the selective delivery molecule of Formula V is: SDM-101, SDM-102, SDM-103, SDM-104, SDM-106, SDM-107, SDM-108, SDM-109, SDM-110, SDM-111, SDM-112, SDM-113, SDM-114, SDM-117, SDM-118, SDM-119, SDM-120, SDM-121, SDM-122, SDM-123, SDM-126, SDM-127, SDM-128, SDM-129, SDM-130, SDM-131, SDM-133, SDM-139, SDM-140, SDM-142, SDM-143, SDM-144, SDM-145, SDM-146, SDM-147, SDM-148, SDM-151, SDM-152, and SDM-153.

42. A pharmaceutical composition comprising a selective delivery molecule conjugate of claim 18 and one or more pharmaceutically acceptable carriers, glidants, diluents, or excipients.

43. A method for treating cancer in a subject in need thereof, comprising administering to the subject a therapeutically effective amount of a selective delivery molecule conjugate of claim 18, thereby treating the cancer.

44. The method of claim 43, wherein the cancer is a breast cancer, colorectal cancer, ovarian cancer, lung cancer, esophageal cancer, pancreatic cancer, gastro-intestinal cancer, squamous cell carcinoma, prostate cancer, melanoma, thyroid cancer, or sarcoma.

45.-46. (canceled)

47. The molecule of claim 18, further comprising a thiol reactive group attached to A or M.

48. The molecule of claim 47, wherein the thiol reactive group is a maliemide.


SEQUENCE LISTING (33 sequences)

SEQ ID NO: 1 - protein, 6 aa, Artificial Sequence
  Description of Artificial Sequence Synthetic peptide
  MOD_RES (4)..(4): S-methyl-(L)-cysteine
PLGCAG

SEQ ID NO: 2 - protein, 6 aa, Artificial Sequence
  Description of Artificial Sequence Synthetic peptide
PLGLAG

SEQ ID NO: 3 - protein, 5 aa, Artificial Sequence
  Description of Artificial Sequence Synthetic peptide
EEEEE

SEQ ID NO: 4 - protein, 9 aa, Artificial Sequence
  Description of Artificial Sequence Synthetic peptide
EEEEEEEEE

SEQ ID NO: 5 - protein, 8 aa, Artificial Sequence
  Description of Artificial Sequence Synthetic peptide
RRRRRRRR

SEQ ID NO: 6 - protein, 9 aa, Artificial Sequence
  Description of Artificial Sequence Synthetic peptide
RRRRRRRRR

SEQ ID NO: 7 - protein, 8 aa, Artificial Sequence
  Description of Artificial Sequence Synthetic peptide
RPLALWRS

SEQ ID NO: 8 - protein, 8 aa, Artificial Sequence
  Description of Artificial Sequence Synthetic peptide
ESPAYYTA

SEQ ID NO: 9 - protein, 6 aa, Artificial Sequence
  Description of Artificial Sequence Synthetic peptide
DPRSFL

SEQ ID NO: 10 - protein, 6 aa, Artificial Sequence
  Description of Artificial Sequence Synthetic peptide
PPRSFL

SEQ ID NO: 11 - protein, 6 aa, Artificial Sequence
  Description of Artificial Sequence Synthetic peptide
RLQLKL

SEQ ID NO: 12 - protein, 5 aa, Artificial Sequence
  Description of Artificial Sequence Synthetic peptide
  C-term acetyl
RLQLK

SEQ ID NO: 13 - protein, 9 aa, Artificial Sequence
  Description of Artificial Sequence Synthetic peptide
  MISC_FEATURE (1)..(9): This sequence may encompass 5-9 residues wherein some positions may be absent
EEEEEEEEE

SEQ ID NO: 14 - protein, 8 aa, Artificial Sequence
  Description of Artificial Sequence Synthetic peptide
  MISC_FEATURE (1)..(8): This sequence may encompass 5-8 residues wherein some positions may be absent
EEEEEEEE

SEQ ID NO: 15 - protein, 7 aa, Artificial Sequence
  Description of Artificial Sequence Synthetic peptide
  MISC_FEATURE (1)..(7): This sequence may encompass 5-7 residues wherein some positions may be absent
EEEEEEE

SEQ ID NO: 16 - protein, 6 aa, Artificial Sequence
  Description of Artificial Sequence Synthetic peptide
EEEEEE

SEQ ID NO: 17 - protein, 7 aa, Artificial Sequence
  Description of Artificial Sequence Synthetic peptide
EEEEEEE

SEQ ID NO: 18 - protein, 8 aa, Artificial Sequence
  Description of Artificial Sequence Synthetic peptide
EEEEEEEE

SEQ ID NO: 19 - protein, 7 aa, Artificial Sequence
  Description of Artificial Sequence Synthetic peptide
RRRRRRR

SEQ ID NO: 20 - protein, 6 aa, Artificial Sequence
  Description of Artificial Sequence Synthetic peptide
  MOD_RES (4)..(4): Any amino acid
PLGXAG

SEQ ID NO: 21 - protein, 10 aa, Artificial Sequence
  Description of Artificial Sequence Synthetic peptide
GGAANLVRGG

SEQ ID NO: 22 - protein, 10 aa, Artificial Sequence
  Description of Artificial Sequence Synthetic peptide
SGRIGFLRTA

SEQ ID NO: 23 - protein, 5 aa, Artificial Sequence
  Description of Artificial Sequence Synthetic peptide
SGRSA

SEQ ID NO: 24 - protein, 4 aa, Artificial Sequence
  Description of Artificial Sequence Synthetic peptide
GFLG

SEQ ID NO: 25 - protein, 4 aa, Artificial Sequence
  Description of Artificial Sequence Synthetic peptide
ALAL

SEQ ID NO: 26 - protein, 5 aa, Artificial Sequence
  Description of Artificial Sequence Synthetic peptide
  MOD_RES (3)..(3): S-ethylcysteine
PICFF

SEQ ID NO: 27 - protein, 8 aa, Artificial Sequence
  Description of Artificial Sequence Synthetic peptide
GGPRGLPG

SEQ ID NO: 28 - protein, 6 aa, Artificial Sequence
  Description of Artificial Sequence Synthetic peptide
HSSKLQ

SEQ ID NO: 29 - protein, 10 aa, Artificial Sequence
  Description of Artificial Sequence Synthetic peptide
LVLASSSFGY

SEQ ID NO: 30 - protein, 10 aa, Artificial Sequence
  Description of Artificial Sequence Synthetic peptide
GVSQNYPIVG

SEQ ID NO: 31 - protein, 10 aa, Artificial Sequence
  Description of Artificial Sequence Synthetic peptide
GVVQASCRLA

SEQ ID NO: 32 - protein, 4 aa, Artificial Sequence
  Description of Artificial Sequence Synthetic peptide
DEVD

SEQ ID NO: 33 - protein, 6 aa, Artificial Sequence
  Description of Artificial Sequence Synthetic peptide
GWEHDG